(19) United States
(12) Patent Application Publication
(10) Pub. No.: US 2023/0285470 A1
(43) Pub. Date: Sep. 14, 2023

(54) A METHOD OF GENERATING AN INDUCED PLURIPOTENT STEM CELL, AN INDUCED PLURIPOTENT STEM CELL AND METHODS OF USING THE INDUCED PLURIPOTENT STEM CELL

(71) Applicants: CELLRESEARCH CORPORATION PTE LTD, Singapore (SG); SINGAPORE HEALTH SERVICES PTE LTD, Singapore (SG)

(72) Inventors: Chou CHAI, Singapore (SG); Kah Leong LIM, Singapore (SG); Toan Thang PHAN, Singapore (SG)

(73) Assignees: CELLRESEARCH CORPORATION PTE LTD, Singapore (SG); SINGAPORE HEALTH SERVICES PTE LTD, Singapore (SG)

(21) Appl. No.: 18/006,136

(22) PCT Filed: Jul. 15, 2021
(86) PCT No.: PCT/SG2021/050412

§ 371 Date: Jan. 19, 2023

Related U.S. Application Data
(60) Provisional application No. 63/054,206, filed on Jul. 20, 2020.

(51) Int. Cl. A61K 35/545; C12N 5/074; A61P 25/16; C12N 15/85; C07K 14/47

(52) U.S. Cl. CPC A61K 35/545; C12N 5/0696; A61P 25/16; C12N 15/85; C07K 14/4702

(57) ABSTRACT

The invention relates to a method of generating an induced pluripotent stem cell. The method of the disclosure comprises expressing exogenous nucleic acids encoding the proteins OCT3/4, SOX2, KLF4, LIN28 and L-MYC and the p53-shRNA in a stem cell of the amniotic membrane of the umbilical cord under conditions suitable to reprogram the stem cell, thereby generating the induced pluripotent stem cell. The present invention also refer to an induced pluripotent stem cell population obtainable by the method and an induced pluripotent stem cell population obtained by the method. Further, a pharmaceutical composition comprising the induced pluripotent stem cell of the present invention is concerned. The present invention also relates to a method of differentiating the induced pluripotent stem cell of this invention. In addition, a pharmaceutical composition comprising a differentiated induced pluripotent stem cell obtained by the method is also concerned. Further, the present invention concerns a method of treating a congenital or acquired degenerative disorder in a subject, comprising administering to a subject a target cell differentiated from pluripotent stem cell.

### CROSS-REFERENCE TO RELATED APPLICATIONS

The present application claims the benefit of priority of U.S. Provisional Application No. 63/054,206 filed Jul. 20, 2020, the content of which is hereby incorporated by reference it its entirety for all purposes.

### BACKGROUND OF THE INVENTION

The present invention relates to a method of generating an induced pluripotent stem cell. In addition, the present invention concerns an induced pluripotent stem cell population obtainable by the method and an induced pluripotent stem cell population obtained by the method. The present invention also relates to a pharmaceutical composition comprising the induced pluripotent stem cell of the present invention. The present invention also relates to a method of differentiating the induced pluripotent stem cell of this invention. In addition, a pharmaceutical composition comprising a differentiated induced pluripotent stem cell obtained by the method is also concerned. Further, the present invention concerns a method of treating a congenital or acquired degenerative disorder in a subject, comprising administering to a subject a target cell differentiated from pluripotent stem cell.

### FIELD OF THE INVENTION

Stem cells are a cell population possessing the capacities to self-renew indefinitely and to differentiate in multiple cell or tissue types. The ability of stem cells to self-renew is critical to their function as reservoir of primitive undifferentiated cells and the “plasticity” of stem cells relies on their ability to trans-differentiate into tissues different from their origin and, perhaps, across embryonic germ layers. In contrast, most somatic cells have a limited capacity for self-renewal due to telomere shortening (reviewed, for example, in Dice, J. F. (1993) Physiol. Rev. 73, 149-159). Stem cell-based therapies thus have the potential to be useful for the treatment of a multitude of human and animal diseases.

Embryonic stem cells (from approximately days 3 to 5 after fertilization) proliferate indefinitely and can differentiate spontaneously into all tissue types: they are thus termed pluripotent stem cells (reviewed, for example, in Smith, A. G. (2001) Annu. Rev. Cell. Dev. Biol. 17, 435-462). Even though the potential of embryonic stem cells is enormous, their use implies many ethical problems. Therefore, non-embryonic stem cells have been proposed as alternative sources.

Adult stem cells are more tissue-specific and may have less replicative capacity: they are thus termed multipotent stem cells (reviewed, for example, in Paul, G. et al. (2002) Drug Discov. Today 7, 295-302). These cells can be derived from the bone marrow stroma, fat tissue and dermis and have the ability to differentiate inter alia into chondrocytes, adipocytes, osteoblasts, myoblasts, cardiomyocytes, astrocytes, and tenocytes. In many cases, however, the number of stem cells extracted from the bone marrow stroma, fat tissue, dermis and umbilical cord blood is rather low.

A comprehensive source for very young and adaptable adult stem cells, also referred to as neonatal stem cells, is the umbilical cord blood or tissue or the placenta. For example, a large amount of stem cells can be derived from umbilical cord tissue, namely from Wharton's jelly, the matrix of umbilical cord (Mitchell, K. E. et al. (2003) Stem Cells 21, 50-60; U.S. Pat. No. 5,919,702; US Patent Application 2004/0136967). These cells have been shown to have the capacity to differentiate, for example, into a neuronal phenotype and into cartilage tissue, respectively. Mesenchymal stem cells have also been isolated from the subendothelial layer of the umbilical cord vein, one of the three vessels (two arteries, one vein) found within the umbilical cord (Romanov, Y. A. et al. (2003) Stem Cells 21, 105-110; Covas, D. T. et al. (2003) Braz. J. Med. Biol. Res. 36, 1179-1183). Further, mesenchymal stem cells as well as epithelial stem cells have successfully been isolated from the amniotic tissue of the umbilical cord (US2006/0078993). Although, for example, mesenchymal stem cells can undergo differentiation in vitro and in vivo, making these stem cells promising candidates for mesodermal defect repair and disease management, the use of adult stem cells is limited by their multipotency. To overcome this limitation, non-embryonic cells can be reprogrammed to pluripotent stem cells: the so called induced pluripotent stem cells (iPS).

IPS were generated for the first time by Takahashi and Yamanaka, who reprogrammed non-embryonic cells to a pluripotent state through overexpression of the four transcription factors OCT3/4, SOX2, KLF4 and C-MYC, also known as Yamanaka factors (Takahashi, K. and Yamanaka, S. (2006), Cell, 126(4), pp. 663-676). In detail, Takahashi and Yamanaka used mouse embryonic fibroblasts and introduced the Yamanaka factors via retroviral transduction, thereby allowing the overexpression of the transcription factors and thus generating cells exhibiting the morphology and growth properties of embryonic cells. Although this method was a major breakthrough, the transduction process may result in an incorporation of the transferred DNA into the genome of the host cells making the iPS critical for therapeutic treatment in humans. A non-integrative alternative to generate iPS has been established in 2011 by Okita, K. et al., Nature methods, 8(5), pp. 409-412. Okita et al. used electroporation to transfer three episomal plasmid vectors encoding the Yamanaka factors and a p53-shRNA for p53 suppression into human dermal fibroblasts and dental pulp, thus, allowing the overexpression of the exogenous DNA and thereby generating integration free human iPS. To support the growth and maintenance of the integration free human iPS, Okita et al., supra, cultivated the iPS on a feeder layer consisting of mouse embryonic fibroblast (MEF) or a STO cell line, which has been transformed with neomycin resistance and murine LIF genes (SNL). The cultivation on a feeder layer, however, may entail the risk of contaminating the iPS with foreign DNA. Thus, the insertion free iPS according to Okita et al., supra, may also be critical for therapeutic treatment in humans.

A decade after its conception, iPS technology has entered the clinical translation stage with first-in-human trials being conducted for age-related macular degeneration (AMD; Mandai, M., et al., N Engl J Med, 2017. 376(11): p. 1038-1046) and Parkinson's Disease (PD; Reardon, S. and Cyranoski, D. (2014) ‘Japan stem-cell trial stirs envy’, Nature. England, pp. 287-288. doi: 10.1038/513287a). The greatest promise of iPS technology lies in its potential for enabling autologous cell therapy, which may circumvent the need for long-term immunosuppression or histocompatibility matching to prevent rejection of transplanted cells. This paradigm has been demonstrated with fibroblasts and bone marrow derived iPS in non-human primate models (Morizane, A., et al., Stem Cell Reports, 2013. 1(4): p. 283-92; Hallett, P. J., et al., Cell Stem Cell, 2015. 16(3): p. 269-74; Wang, S., et al., Cell Discov, 2015. 1: p. 15012; Shiba, Y., et al., Nature, 2016. 538(7625): p. 388-391), and is the basis of the first human trial of iPS-based cell therapy for AMD (Mandai, M., et al., N Engl J Med, 2017. 376(11): p. 1038-1046). However, the significant period of time and costs associated with the production of clinical-grade iPS will make it unlikely to be implemented on a large-scale for human therapy. In addition, circumstances exist where the generation of autologous iPS from a patient may not be practical. For instance, for patients carrying disease-causing mutations, it is first necessary to correct these mutations before iPS derived from these patients can be used. This is achievable when the mutations are tractable but in cases where the mutations are intractable, such as those underlying the sporadic form of many diseases, a gene correction strategy may not be tenable.

Accordingly, there is still a need for an alternative method to generate iPS, wherein the resulting iPS are capable to differentiate into a target cell suitable for therapeutic treatment in humans. Consequently, it is an object of the invention to provide a method of generating and differentiating iPS that meet these needs.

### SUMMARY OF THE INVENTION

The invention relates to a method of generating an induced pluripotent stem cell as described herein, a resulting induced pluripotent stem cell, a method of differentiating a resulting induced pluripotent stem cell and a method of treating a disorder in a subject with a differentiated cell derived from an induced pluripotent stem cell.

In a first aspect, the invention provides a method of generating an induced pluripotent stem cell, wherein the method comprises expressing exogenous nucleic acid encoding the proteins OCT3/4, SOX2, KLF4, LIN28 and L-MYC and the p53-shRNA in a stem cell of the amniotic membrane of the umbilical cord under conditions suitable to reprogram the stem cell, thereby generating the induced pluripotent stem cell. In embodiments of this method stem cell of the amniotic membrane of the umbilical cord is a mesenchymal stem cell of the amniotic membrane of the umbilical cord or an epithelial stem cell of the amniotic membrane of the umbilical cord.

In a second aspect, the invention also provides an induced pluripotent stem cell population obtainable by the method as well as an induced pluripotent stem cell population obtained by the method. The induced pluripotent stem cell population can either be an induced pluripotent stem cell population that is derived from a mesenchymal stem cell (population) of the amniotic membrane of umbilical cord or an induced pluripotent stem cell population that is derived from an epithelial stem cell (population) of the amniotic membrane of the umbilical cord.

In a third aspect, the invention also provides a pharmaceutical composition comprising an induced pluripotent stem cell of the present invention.

In a fourth aspect, the invention provides a method of differentiating an induced pluripotent stem cell of the present invention into a target cell, wherein the induced pluripotent stem cell is differentiated into the target cell under conditions suitable for differentiation. Consequently, the invention also provides a pharmaceutical composition comprising a differentiated induced pluripotent stem cell obtained by the present invention.

In a fifth aspect, the invention provides a method of treating a congenital or acquired degenerative disorder in a subject, comprising administering to a subject a target cell differentiated from a pluripotent stem cell obtained by the present invention.

In a sixth aspect, the invention provides an extracellular membranous vesicle produced by an induced pluripotent stem cell population of the invention or produced by a cell obtained by differentiation of an induced pluripotent stem cell of the invention. This sixth aspect further comprises the use of such a extracellular membranous vesicle of the invention as delivery carrier of a therapeutic agent.

In a seventh aspect, the invention provides a cell culture medium comprising Mammary Epithelial Basal Medium MCDB 170, EpiLife medium, DMEM (Dulbecco's modified eagle medium), F12 (Ham's F12 Medium) and FBS (Fetal Bovine Serum).

### BRIEF DESCRIPTION OF THE DRAWINGS

The invention will be better understood with reference to the detailed description when considered in conjunction with the non-limiting examples and the drawings, in which:

FIG. 1 shows a flow-diagram schematically representing the experimental steps of an illustrative embodiment of a method of generating an induced pluripotent stem cell of the present invention. The stem cells used herein are isolated from the amniotic membrane of the umbilical cord—also referred to as cord lining stem cells (CLSC). This embodiment starts with the harvesting of isolated CLSC by dissociating the cells from the cell culture device (it is however to be noted here that the CLSC can also be supplied for the method of the invention in isolated form). Then, the CLSC are counted and about 0.7 million cells are aliquoted into a microfuge and pelleted. The cells pellet is resuspended in a buffer suitable for electroporation before the plasmids encoding for the Yamanaka factors are added to the cells-buffer mixture. The electroporation is carried out with 1 pulse having a duration time of about 20 ms and a voltage of about 1600V or with 2 pulses having a duration time of 30 ms and a voltage of about 1350V for cord lining mesenchymal cells (CLMC) and cord lining epithelial cells (CLEC), respectively. After electroporation, the stem cells are immediately transferred into a medium suitable for recovery, wherein the medium contains a compound suppressing inflammatory response and enhancing cell survival. After a suitable time of recovery, the medium suitable for recovery is replaced with a 1:1 mixture of two different cell culture media, wherein the two different cell culture media are the medium suitable for recovery and a second cell culture medium. To refresh the cell culture medium, the media mixture is replaced with the same mixture of cell culture media about 4 days after electroporation. Thereby, colonies of cord lining induced pluripotent stem cells—also referred herein to as CLiPS—are generated. After about 2 further days, the 1:1 mixture of two different cell culture media is replaced with the second cell culture medium. This medium is also replaced about every second day to keep the medium fresh. When reaching a size of about 0.5 mm to 1.5 mm in diameter, the CLiPS colonies are picked and transferred to a coated cell culture vessel suitable for cell cultivation and proliferation. Again, the cell culture medium is replaced regularly with the same medium. After reaching a confluence of about 50%, the CLiPS colonies are detached from the coated culture device and transferred to another cell culture vessel suitable for cell cultivation and proliferation. This way, the CLiPS colonies are further dissociated. When reaching a confluence of about 70-80%, the CLiPS are passaged in a ratio of about 1:3 (v/v), wherein the passaging in a ratio of about 1:3 (v/v) is performed by contacting 1 volume dissociated CLiPS to 2 volume of fresh culture medium. The CLiPS are then cultivated in a medium containing a substance enhancing the survival of the cells until reaching a confluence of about 30-60%. At this point, the CLiPS are capable to be differentiated into any desired target cells.

FIG. 2 shows an exemplary comparison of the reprogramming efficiency of individual CLSC populations. The stem cells have been subjected to different electroporation settings to transfect the exogenous nucleic acid into the cells. The electroporation has been carried out using the electroporation parameters indicated in Okita et al, supra, (1650V, 10 ms, 3 pulses) and the respective parameters used in the present invention for transfection of epithelial stem cells of the amniotic membrane of the umbilical cord (also referred herein as “cord lining epithelial stem cell” or CLEC, 1350V, 30 ms, 2 pulses) and mesenchymal stem cells of the amniotic membrane of the umbilical cord (also referred herein to as cord lining mesenchymal stem cell or CLMC, (1600V, 20 ms, 1 pulse), respectively. 200K transfected cells were plated in triplicates in 6-well plates. About 21 days after transfection, the percentage reprogramming efficiency has been calculated as Colony number/200,000×10.

FIG. 3 shows exemplary colony development of induced pluripotent stem cells from human CLMC. FIG. 3a-f show a representative time course of colony development, wherein FIG. 3a depicts a typical morphology of human CLMC cultured in its maintenance medium at Day 0 of cultivation. FIG. 3b depicts a typical morphology of human CLMC cultured in its maintenance medium at Day 15 of cultivation. FIG. 3c depicts a typical morphology of human CLMC cultured in its maintenance medium at Day 24 of cultivation. FIG. 3d depicts a typical morphology of human CLMC cultured in its maintenance medium at Day 29 of cultivation. FIG. 3e shows a 4× magnification of the typical morphology of an iPS colony first passage and 3f shows a 10× magnification of the typical morphology of an iPS colony at first passage. FIG. 3g-l depict an exemplary immunofluorescence staining of iPS derived from human cord lining cells showing the activation of endogenous expression of pluripotent embryonic stem cell markers, wherein FIG. 3g shows the expression of KLF4, FIG. 3h shows the expression of NANOG, FIG. 3i shows the expression of OCT3/4, FIG. 3j shows the expression of SOX2, FIG. 3k shows the expression of SSEA4 and FIG. 3l shows the expression of Tra-1-60. FIG. 3m shows an exemplary karyotype analysis demonstrating normal chromosomal numbers and G-banding patterns of CLiPS in the individual cell lines CLEC23 (EC23-CLiPS), CLMC23 (MC23-CLiPS), CLEC44 (EC44-CLiPS) and CLMC44 (MC44-CLiPS). FIG. 3n shows an exemplary human CLMSC-DTHN culture emerging 10 days of reprogramming magnified 20× FIG. 3o shows exemplary a morphology of expanding human CLMSC-DTHN cultured on laminin-511 substrate magnified 4×. FIG. 3p shows exemplary a morphology of expanding human CLMSC-DTHN cultured on laminin-511 substrate magnified 10×. FIG. 3q shows exemplary a morphology of expanding human CLMSC-DTHN cultured on laminin-511 substrate magnified 20×. FIG. 3r shows an exemplary expression of the human pluripotent marker NANOG in CLMSC-DTHN iPS at passage No. 3. FIG. 3s shows an exemplary expression of the human pluripotent marker OCT3/4 in CLMSC-DTHN iPS at passage No. 3. FIG. 3t shows an exemplary expression of the human pluripotent marker SOX2 in CLMSC-DTHN iPS at passage No. 3. FIG. 3u shows an exemplary expression of the human pluripotent marker NTRA-1-81 in CLMSC-DTHN iPS at passage No. 3. Scale bars: all 100 μm. FIG. 3v shows an exemplary RT-PCR analyses of reprogramming gene expression and pluripotent gene expression in primary parental cells, parental cells 11 days after vector transfection (D11 transfected cells) and in established iPS clones (CLiPS). ‘Vec’ denotes amplification specific for vector derived sequences. Glycerinaldehyd-3-phosphat-Dehydrogenase (GAPDH) was used as an internal control. PCR of Homo sapiens (H1) total RNA without reverse transcription was used to control for genomic contamination for all primer pairs.

FIG. 4 shows an exemplary histological analysis of a teratoma formed by immunocompromised non-obese diabetic severe combined immunodeficiency (NOD-SCID) mice after CLiPS injection. The teratoma formation assay reveals the formation of all three germ layers. FIG. 4a-inset shows a teratoma obtained from human CLEC-derived iPS 3 months after subcutaneous injection. Sections of the teratoma are further analyzed by a hematoxylin and eosin staining FIG. 4a shows the presence of respiratory-like epithelium in the teratoma. FIG. 4b shows the presence of glandular structures representing the endoderm in the teratoma. In FIG. 4c the arrowhead shows the presence of cartilage in the teratoma. In FIG. 4d the arrowhead shows the presence of bone representing the mesoderm in the teratoma. FIG. 4e shows the presence of renal tissue in the teratoma. The filled arrowheads indicate glomeruli and the hollow arrowheads indicate renal tubules.

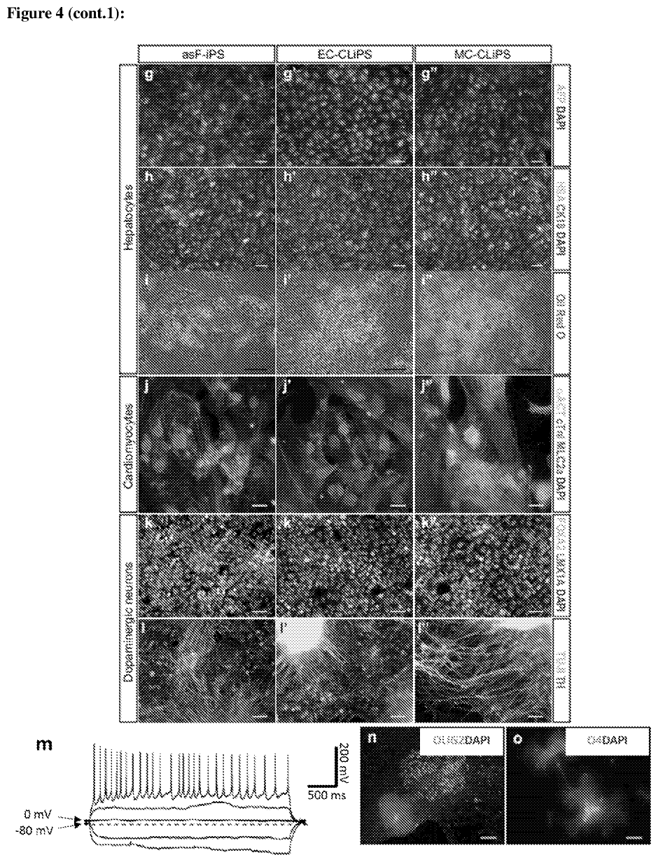

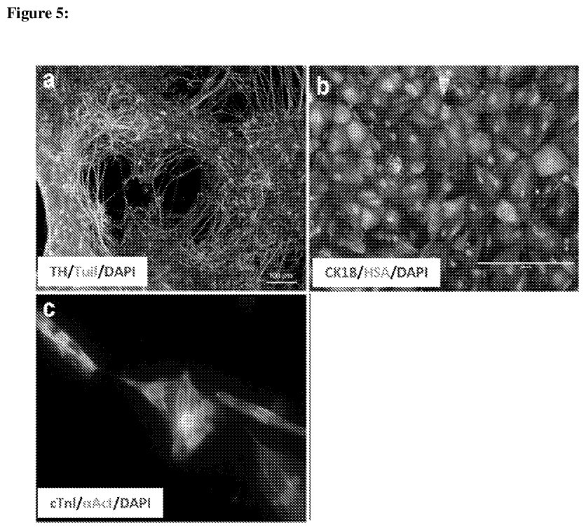

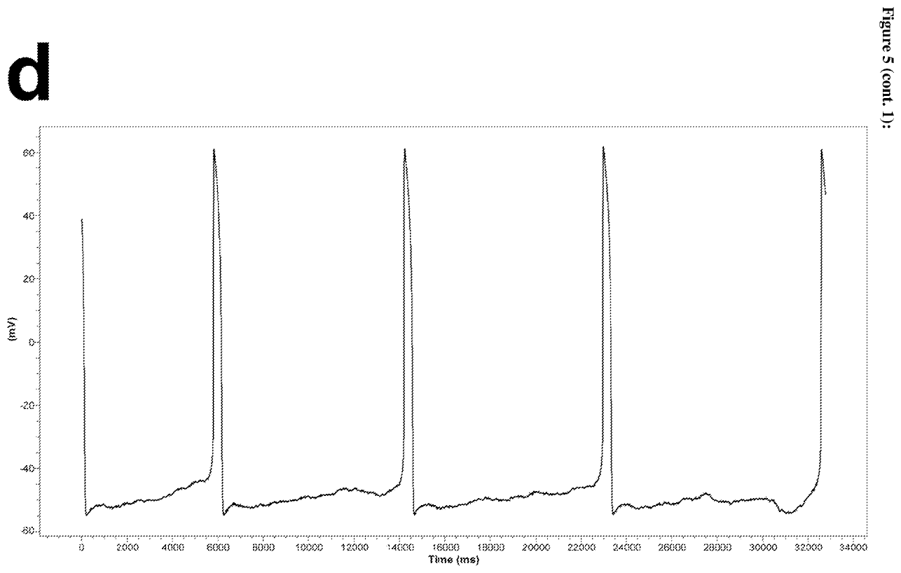

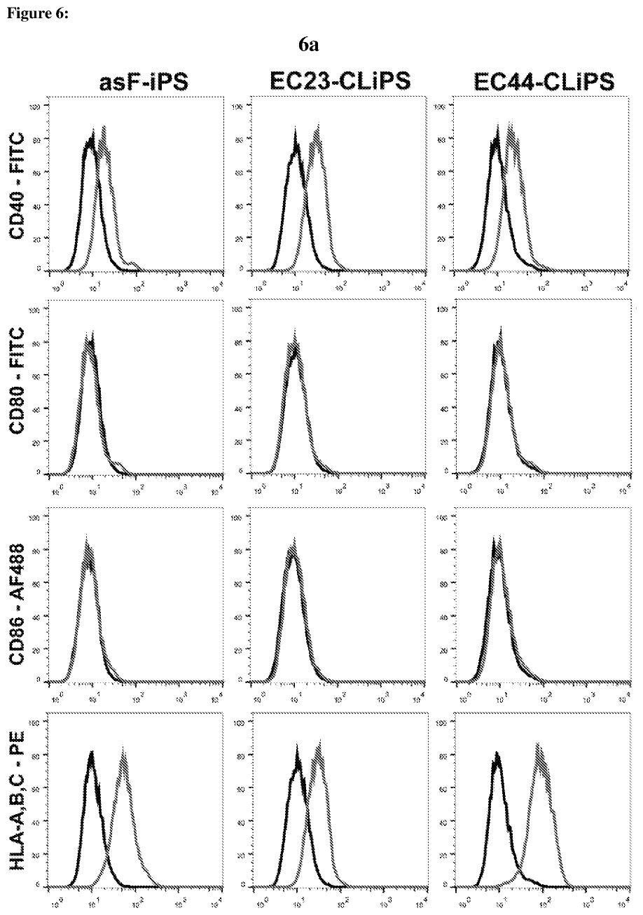

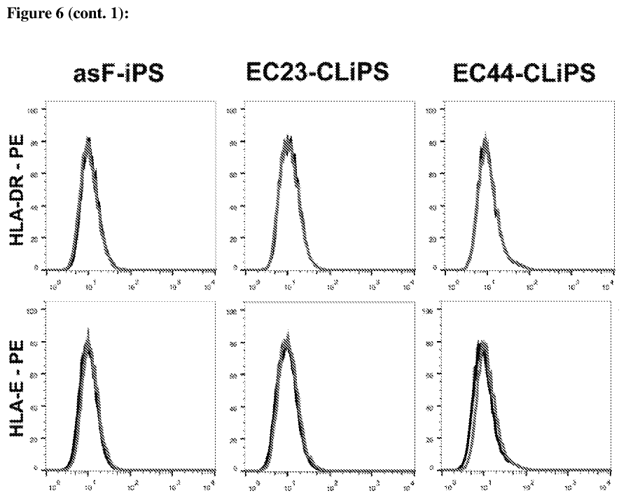

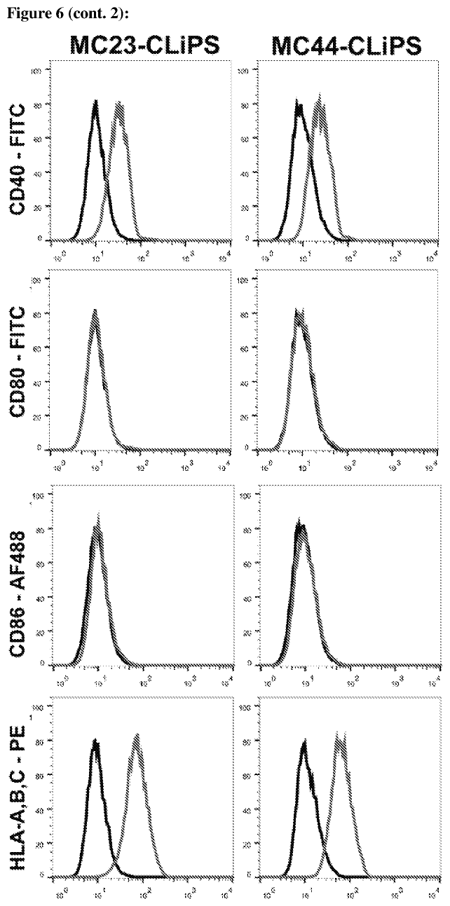

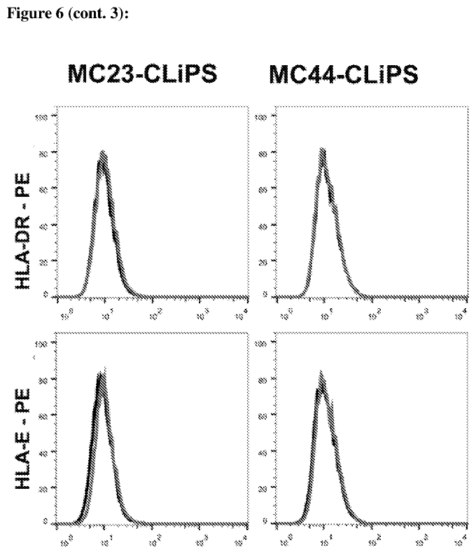

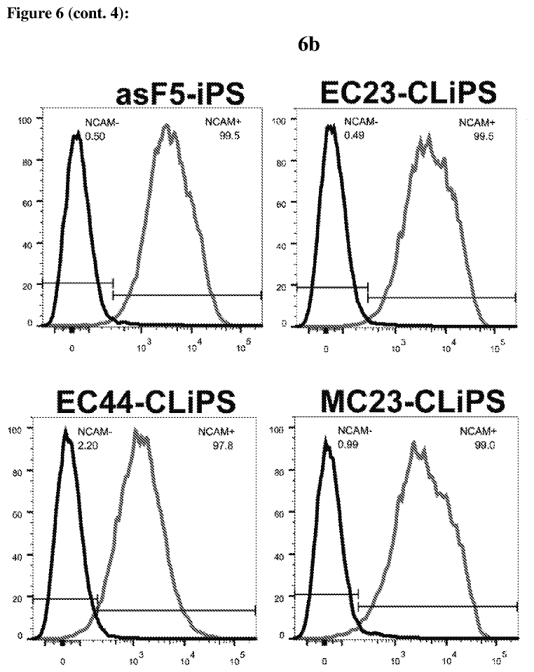

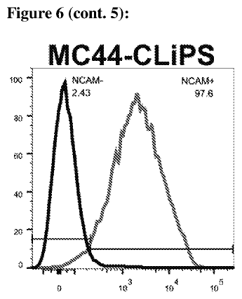

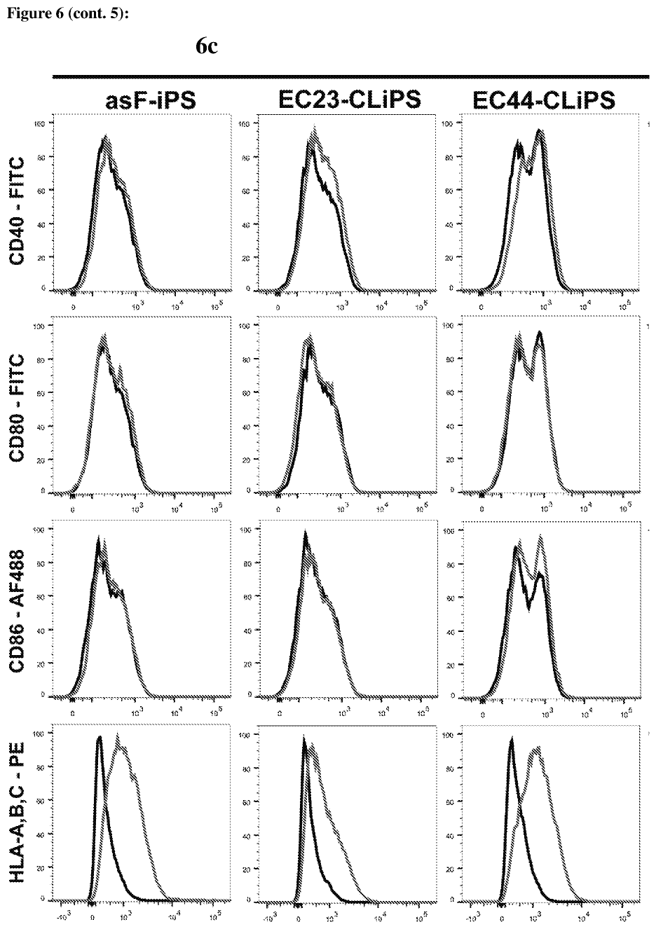

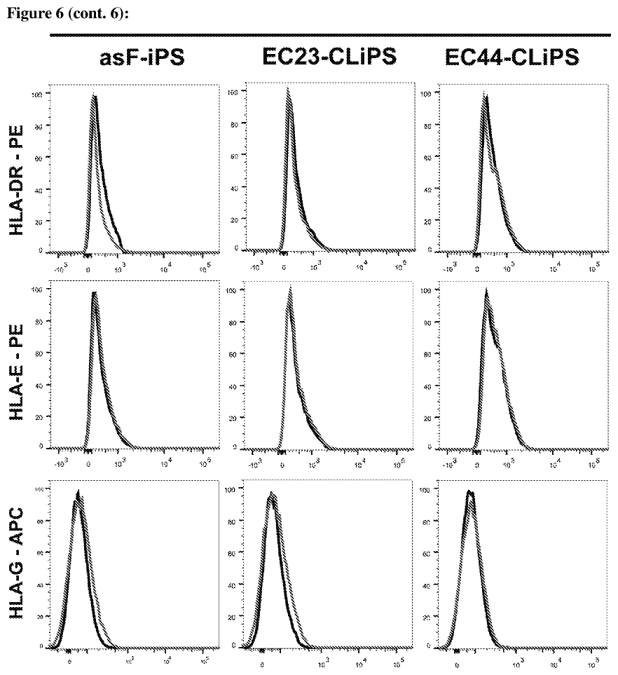

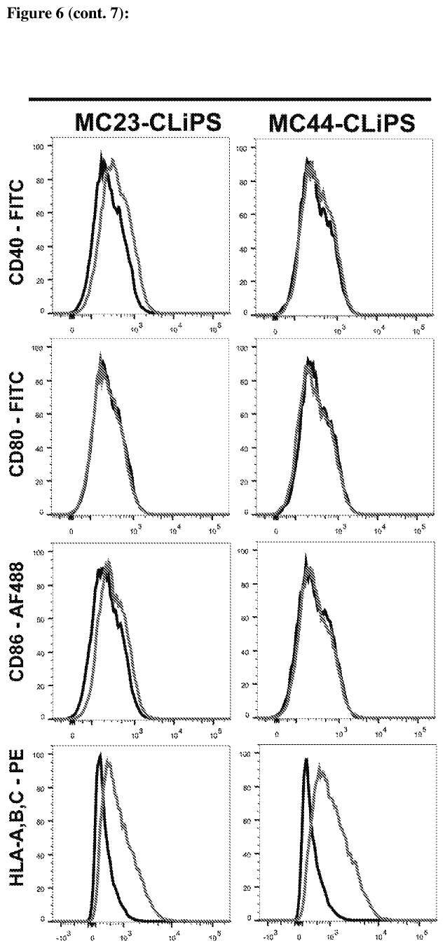

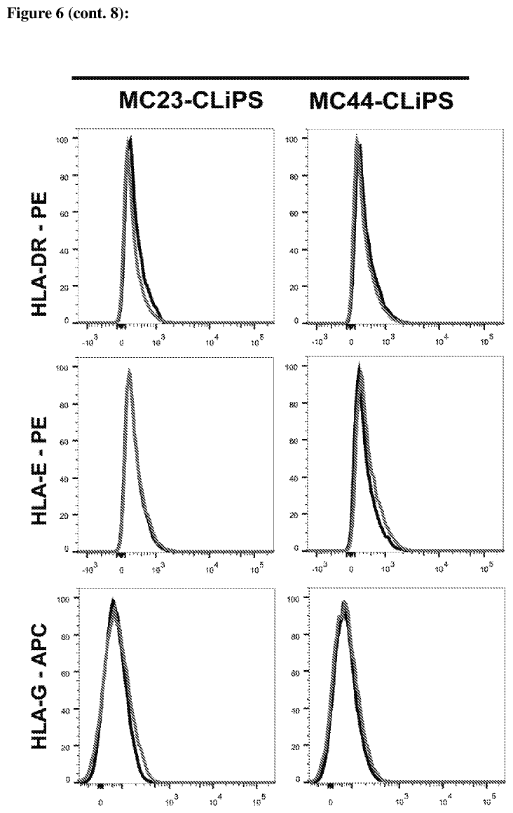

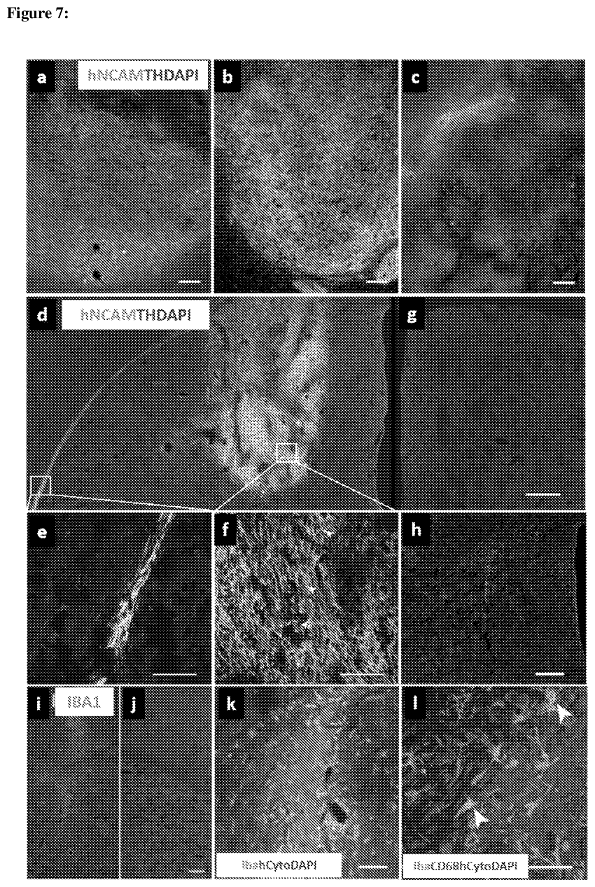

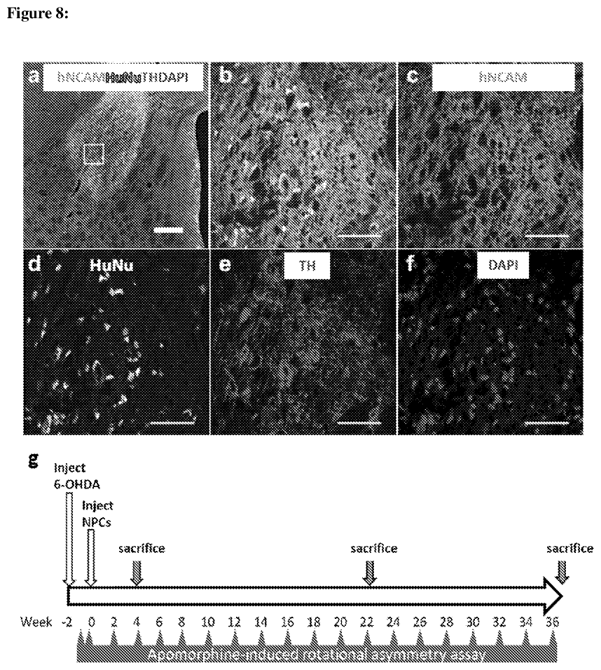

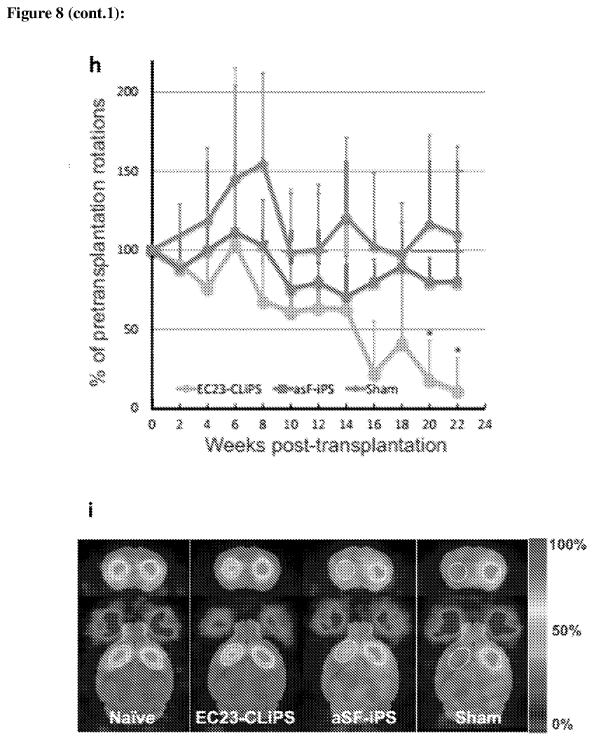

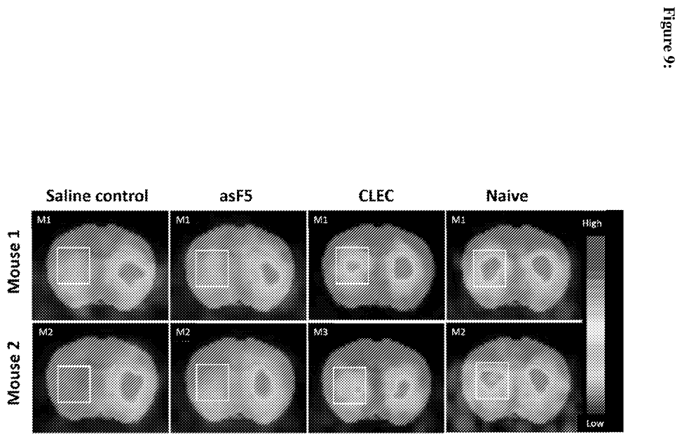

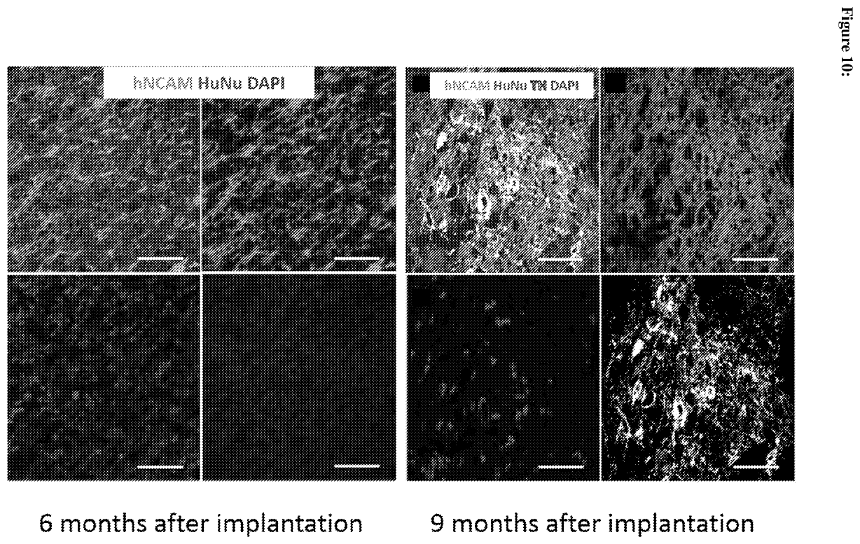

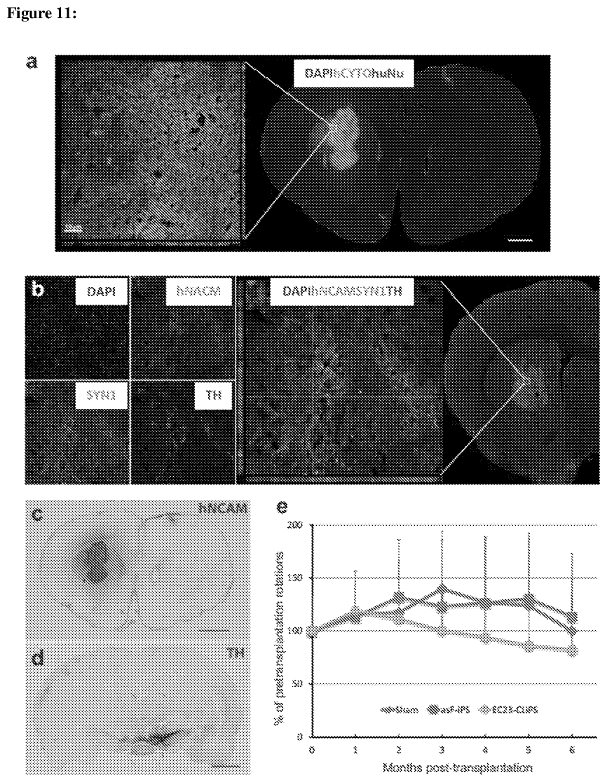

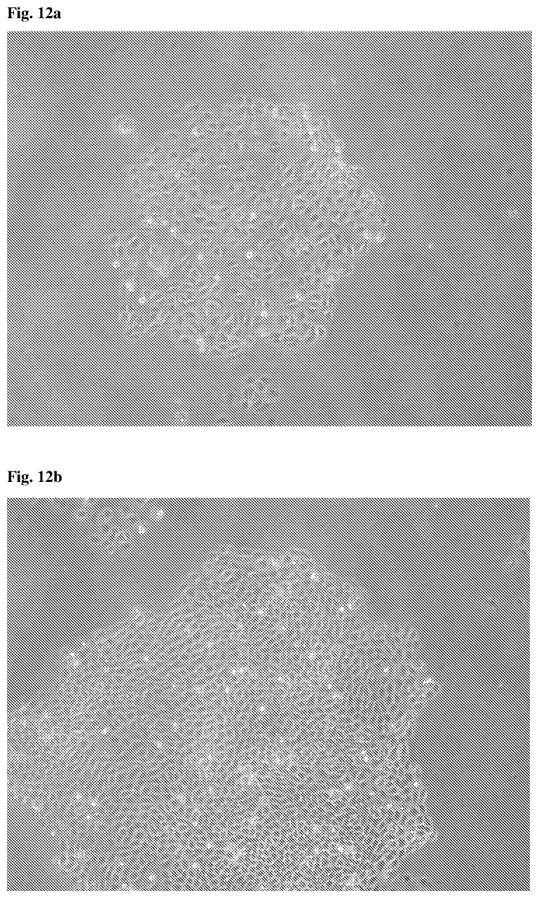

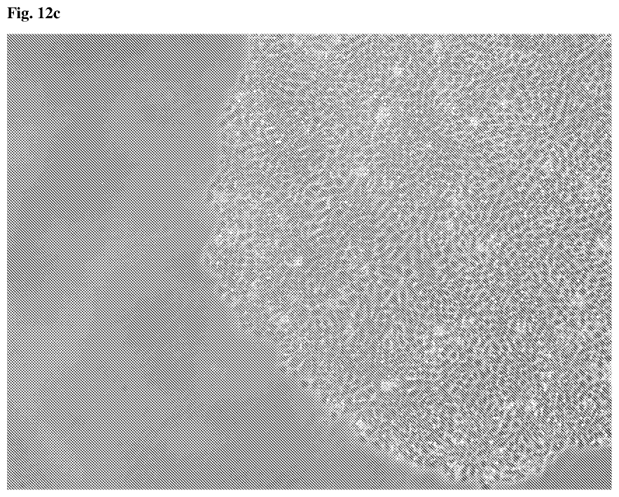

In FIG. 4f the arrowhead shows the presence of neural epithelium representing the ectoderm in the teratoma. Using directed differentiation protocols, CLiPS were induced to differentiate into specific tissues. FIG. 4g shows CLiPS differentiated into hepatocytes visualized with alpha-fetoprotein (AFP) and 4′,6-diamidino-2-phenylindole (DAPI). FIG. 4h shows CLiPS differentiated into hepatocytes visualized with human serum albumin (HAS), cytokeratin 18 (CK18) and DAPI. FIG. 4i shows CLiPS differentiated into hepatocytes visualized with Oil Red O. FIG. 4j shows CLiPS differentiated into cardiomyocytes visualized with alpha-actinin (αACT), cardiac troponin I (cTn1), myosin regulatory light chain 2a (MLC2a) and DAPI. FIG. 4k shows CLiPS differentiated into dopaminergic neurons visualized with the floor-plate marker FOXA2, the roof plate marker LMX1A and DAPI. FIG. 4l shows CLiPS differentiated into dopaminergic neurons visualized with neuron-specific class III beta-tubulin (TUJI) and tyrosine Hydroxylase (TH). FIG. 4m shows CLiPS differentiated into oligodendrocyte progenitor cells visualized with OLIG2 and DAPI. FIG. 4n shows CLiPS differentiated into oligodendrocyte progenitor cells visualized with 04 and DAPI. FIG. 4o shows an electrophysiological analysis of mature human CLiPS-derived dopaminergic neurons at Day 45 of differentiation. The human CLiPS-derived dopaminergic neurons fire trains of action potential with injected currents. Scale bars: 200 μm in FIG. 4a, FIG. 4c and FIG. 4d; 100 μm in FIG. 4b, FIG. 4e and FIG. 4f; 50 μm in FIG. 4g, FIG. 4h, FIG. 4i, FIG. 4k, FIG. 4l, FIG. 4m; 25 μm in FIG. 4j, FIG. 4n.

FIG. 5 shows an exemplary directed differentiation of human CLiPS into various different cell types, wherein FIG. 5a depicts human CLiPS-derived neurons visualized with TH, Tuik and DAPI, FIG. 5b depicts human CLiPS-derived hepatocytes visualized with CK18, HAS and DAPI, FIG. 5c depicts human CLiPS-derived cardiomyocytes visualized with cTn1, αAct and DAPI and FIG. 5d shows an electrophysiological analysis of contracting human CLiPS-derived cardiomyocytes illustrating the cells generating spontaneous action potentials.

FIG. 6 shows an exemplary flow cytometric analysis of major histocompatibility complex (MHC) Class I and II, and T-cell co-stimulatory protein expression on iPS and dopaminergic neuroprogenitors differentiated from them. FIG. 6a shows a flow cytometric profile of immune-related gene expression on undifferentiated iPS. FIG. 6b shows a flow cytometric analysis of neural cell adhesion molecule (NCAM)-positive populations. These populations were gated for an analysis of immune-related protein expression. FIG. 6c shows an analysis of immune-related protein expression on Day 25 differentiated dopaminergic neuroprogenitors.

FIG. 7 shows an in vivo comparison of engraftment of dopaminergic neuronal progenitor cells (NPCs) derived from human CLiPS and human adult fibroblast-iPS (asF-iPS) in NOD-SCID mice. The day 25 dopaminergic NPCs were injected into the striatum of NOD-SCID mice to assess the engraftment and differentiation potential of the cells in an immune-deficient environment. TH-immunoreactive dopaminergic neurons are present among abundant human NCAM-positive engrafted neurons. FIG. 7a shows in vivo engraftment of day 25 dopaminergic NPCs derived from human asF-iPS. FIG. 7b shows in vivo engraftment of day 25 dopaminergic NPCs derived from human CLEC-iPS (EC23-CLiPS). FIG. 7c shows in vivo engraftment of day 25 dopaminergic NPCs derived from CLMC-iPS (MC23-CLiPS). FIG. 7d shows an antibody staining of the grafted hemisphere of a Parkinson's Disease (PD) mouse model created in an immunocompetent C57BL/6NTac mouse 1 month after transplantation with human CLEC-iPS-derived dopaminergic NPCs. Human NCAM (green) and TH (red) double positive neurons are present in abundance in the injected site. FIG. 7e shows long neuronal processes originating from the graft site projected along the forceps major of the corpus callosum to distal regions of the brain. Arrowheads in FIG. 7f indicate the human NCAM and TH double positive neurons, which are present in abundance in the injected site, as shown by the arrowheads. FIG. 7g shows the contralateral non-transplanted hemisphere of the same section as shown in FIG. 7d. FIG. 7h illustrates that no surviving cells are visible in striatum transplanted with human adult asF-iPS-derived NPCs suggesting immune rejection. FIG. 7i indicates abundant microglia/macrophage aggregation in the transplanted hemisphere. FIG. 7j shows absence of microglia/macrophage aggregation in the non-transplanted hemisphere. FIG. 7k shows a higher magnification of FIG. 7i. It can be seen that microglia located proximal to and inside graft display a more amoeboid morphology characteristic of activated microglia. FIG. 7l shows a higher magnification of FIG. 7k indicating an expression of CD68, which is an activation marker for microglia. Scale bars: 100 μm in FIG. 7a-c and FIG. 7k; 200 μm in FIG. 7d, FIG. 7g and FIG. 7h; 50 μm in FIG. 7e, FIG. 7f and FIG. 7l.

FIG. 8 shows the survival of human CLEC derived (EC23-CLiPS) dopaminergic neurons in mouse PD model 9 months after transplantation. FIG. 8a indicates HuNu+/hNCAM+/TH+ neurons present in the transplanted hemisphere. FIG. 8b is an overlay of FIGS. 8c-f and shows a higher magnification of the boxed area in FIG. 8a. FIG. 8c indicates hNCAM+ neurons present in the transplanted hemisphere. FIG. 8d indicates HuNu+ neurons present in the transplanted hemisphere. FIG. 8e indicates TH+ neurons present in the transplanted hemisphere. FIG. 8f indicates nuclei of the neurons present in the transplanted hemisphere. FIG. 8g illustrates schematically the experimental steps starting from the induction of PD lesion by 6-hydroxydopamine (6-OHDA) injection into the striatum of C57BL/6NTac mice. Pre-transplantation rotation behavioral assays were performed one and two weeks prior to NPC transplantation. FIG. 8h shows the results of an Apomorphine-induced rotational asymmetry assay in mice transplanted with dopaminergic NPCs derived from human EC23-CLiPS and asF-iPS, and sham control. The assays were performed every two weeks up to 22 weeks after transplantation Animals in the human EC23-CLiPS group showed statistically significant rotational recovery compared to the asF-iPS group beginning from week 20 post-transplantation (n=5, p<0.05). No recovery was observed in sham group. FIG. 8h shows a representative in vivo Positron Emission Tomography (PET) imaging of the uptake of [18F]PE-P2I ligand to evaluate recovery of dopamine transporter (DAT) function in striatal dopaminergic neurons 6 months following transplantation. Mice transplanted with human EC23-iPS NPCs showed recovery of DAT activity compared to those transplanted with human asF-iPS NPCs or sham controls. Scale bars: 200 μm in FIG. 8a; 100 μm in FIG. 8b-f.

FIG. 9 shows an exemplary in vivo PET imaging of striatal dopamine production in engrafted mice. The PET illustrates the uptake of [18F]PE-P2I ligand to evaluate recovery of dopamine transporter (DAT) function in striatal dopaminergic neurons 6 months after iPS-derived NPCs transplantation. Mice transplanted with human CLEC-iPS-derived NPCs show apparent recovery of DAT activity compared to those transplanted with human adult iPS-derived NPCs or sham transplanted controls.

FIG. 10 illustrates the in vivo maintenance of graft derived from human CLiPS 6 and 9 months after implantation into mice brains. The graft is stained positive for human antigen NCAM and TH dopaminergic marker. A formation of tumors has not been recorded. Scale bars: 50 μm.

FIG. 11 shows the results of a histological and functional analysis of transplanted human EC23-CLiPS dopaminergic NPCs in a Medial Forebrain Bundle (MFB) lesion model of PD created in fully-immunocompetent Wistar Hannover rats. FIG. 11a shows engraftment of human EC23-CLiPS neurons in striatal region of a rat brain 3 months after transplantation demonstrated by positive double-staining for human cytoplasm (STEM 121) and human nuclear antigen (HuNu) antibodies. The staining indicates functional recovery. FIG. 11b indicates colocalization of Synapsin 1 immunoreactivity with hNCAM+/TH+ neurons suggesting possible integration of transplanted human CLiPS-derived cells with host tissues 3 months after transplantation. FIG. 11e shows a retrograde lesioning of the dopaminergic system in the substantia nigra in a rat brain. FIG. 11d shows an unlesioned rat brain confirming the retrograde lesioning of the dopaminergic system in the substantia nigra of FIG. 11e by tyrosine hydroxylase (TH) immunostaining FIG. 11e shows the result of an Apomorphine-induced rotational asymmetry assay in rats transplanted with dopaminergic NPCs derived from human CLEC23-iPS. The results indicate that transplantation of CLiPS-NPCs mediated recovery of functional motor deficits in a rat MFB model of PD over a 6 months study period. Scale bars: 100 μm in FIG. 11a and FIG. 11b; 200 μm in FIG. 11c and FIG. 11d.

FIGS. 12a, b and c each show exemplary colonies of induced pluripotent stem cells derived from human CLEC that have been generated by using the medium PTTe-3 as recovery medium.

### DETAILED DESCRIPTION OF THE INVENTION

The present invention is directed to a method of generating an induced pluripotent stem cell, from a stem cell of the amniotic membrane of the umbilical cord under conditions suitable to reprogram the stem cell, thereby generating the induced pluripotent stem cell (iPS).

In the present invention, both mesenchymal and epithelial stem cells of the amniotic membrane of the umbilical cord—also jointly referred to herein as cord lining stem cells (CLSC) are used to generate iPS—also referred to herein as cord lining-derived induced pluripotent stem cells or “CLiPS”. It has been surprisingly found that cord lining-derived induced pluripotent stem cells of the present invention are robust and homogenous stem cells capable to differentiate into functional target cells of different lineages (cf., Examples 3 and 4). For example, cord lining-derived induced pluripotent stem cells have the capacity to differentiate in multiple cell types and can, for example, be differentiated into various cells types such as hepatocytes representing endodermal tissue (cf., Example 8), cardiomyocytes representing mesodermal tissue (cf., Example 9), and dopaminergic neurons (cf., Example 7) and oligodendrocytes (cf., Example 10) representing ectodermal tissue. Even more surprising and important is the finding that, for example, human CLiPS-derived dopaminergic neurons are able to engraft functionally in different species and survived for up to 9 months in mice Parkinson's disease (PD) models in the absence of immunosuppression and 6 months in rat PD models in the absence of immunosuppression (cf. Examples 12 and 13). Therefore, in summary, the present inventors have generated a hypo-immunogenic cell source that is capable of engrafting, integrating and mediating therapeutic recovery in a fully immunocompetent host. The cord lining-derived induced pluripotent stem cells of the present invention can potentially be used as a universal source of cell for allogeneic cell transplantation in humans without the need for immunosuppression, and this making them ideal candidates for such cell based therapies. As a further advantage it was found here that the cord lining-derived induced pluripotent stem cells of the invention can be generated by an integration- and feeder free method, thereby allowing an iPS production under current good manufacturing practice (cGMP) conditions. Since a GMP process for the production of large amounts of mesenchymal stem cells of the amniotic membrane of the umbilical cord has recently been established (see International Application WO 2018/067071 or US patent application US2018127721), the present invention provides an ideal platform to produce iPS for subsequent cell-based therapy in humans or animals.

Describing now first the method of generating an iPS of the present invention, this method may comprise expressing exogenous nucleic acid encoding the proteins OCT3/4, SOX2, KLF4, LIN28 and L-MYC and the p53-shRNA. The nucleic acid encoding OCT3/4 (Sequence ID No: 1), also sometimes referred to as POU5FL, OCT3 or OCT4, encodes for the octamer-binding transcription factor 4. OCT3/4 (Sequence ID No: 2) forms a heterodimer with SOX2 to regulate pluripotency factors in a cell. SOX2 (Sequence ID No: 3), also sometimes referred to as SEY, encodes for the sex determining region Y-box 2 transcription factor (Sequence ID No: 4). When bound to OCT3/4, SOX2 binds to a non-palindromic genomic sequence thus activating the transcription of pluripotent factors in a cell. KLF4 (Sequence ID No: 5), also sometimes referred to as GKLF, encodes for the Krueppel-like factor 4. KLF4 (Sequence ID No: 6) is a zinc finger transcription factor, which functions as a tumor suppressor controlling the G1-to-2 transition of the cell cycle by mediating the tumor suppressor p53. L-MYC (Sequence ID No: 7) encodes for a transcription factor (Sequence ID No: 8) activating the expression of proliferative genes. LIN28 (Sequence ID No: 9) encodes for the RNA-binding protein Lin-28 homolog A (Sequence ID No: 10), which regulates the self-renewal of stem cells. The p53-shRNA (Sequence ID No: 11) encodes for a small hairpin RNA directed to p53, a protein that may regulate the cell cycle by stopping it when the protein accumulates in the cell. To avoid a stopping of the cell cycle by p53, p53-shRNA may silence the expression of p53 posttranscriptional. To generate CLiPS, the exogenous nucleic acids encoding OCT3/4, SOX2, KLF4, LIN28, L MYC and p53-shRNA may be transferred into the CLSC for expression. Alternatively, the proteins OCT3/4, SOX2, KLF4, LIN28, L-MYC and the p53 shRNA may be transferred directly into a CLSC.

As explained above, an induced pluripotent stem cell population of the present invention is obtainable by reprogramming stem cells of the amniotic membrane of umbilical cord. The stem cell of the umbilical cord may be an (isolated) mesenchymal stem cell of the amniotic membrane of the umbilical cord, also referred to as cord lining mesenchymal stem cell (CLMC), or an (isolated) epithelial stem cell of the amniotic membrane of the umbilical cord, also referred to as cord lining epithelial stem cell (CLEC). The CLEC and CLMC used to generate the iPS of the present invention may be derived of any mammalian species, such as mouse, rat, guinea pig, rabbit, goat, horse, dog, cat, sheep, monkey or human, with stem cells of human origin being preferred in one embodiment. Accordingly, also the iPS of the present invention can be derived of any mammalian species, such as mouse, rat, guinea pig, rabbit, goat, horse, dog, cat, sheep, monkey or human, with stem cells of human origin being preferred in one embodiment.

In case epithelial stem cells of the amniotic membrane of the umbilical cord are used as starting material, these epithelial stem cells, can, for example, be obtained as described in US patent application 2006/0078993 (leading to granted U.S. Pat. Nos. 9,085,755 and 9,737,568) or the corresponding International patent application WO2006/019357. If mesenchymal stem cells of the amniotic membrane of the umbilical cord are used as starting material, they can also be obtained as described in US patent application 2006/0078993 (leading to U.S. Pat. Nos. 9,085,755 and 9,737,568) or the corresponding International patent application WO2006/019357.

It is also possible to use as starting material, a mesenchymal stem cell population as described in the published US application 2018/127721 or the corresponding International Application WO 2018/067071. The mesenchymal stem cell population of International Application WO 2018/067071 has the advantage that 99% or more of the stem cells of this population are positive for the three mesenchymal stem cell markers CD73, CD90 and at the same lack expression of CD34, CD45 and HLA-DR, meaning 99% or even more cells of the mesenchymal stem population International Application WO 2018/067071 express the stem cell markers CD73, CD90 and CD105 while not expressing the markers CD34, CD45 and HLA-DR. This extremely homogenous and well defined cell population is the ideal candidate for clinical trials and cell based therapies since, they for example, fully meet the criteria generally accepted for human mesenchymal stem cells to be used for cellular therapy as defined, for example, by Dominici et al, “Minimal criteria for defining multipotent mesenchymal stromal cells. The International Society for Cellular Therapy position statement”, Cytotherapy (2006) Vol. 8, No. 4, 315-317, Sensebe et al., “Production of mesenchymal stromal/stem cells according to good manufacturing practices: a, review”, Stem Cell Research & Therapy 2013, 4:66), Vonk et al., Stem Cell Research & Therapy (2015) 6:94, or Kundrotas Acta Medica Lituanica. 2012. Vol. 19. No. 2. P. 75-79. Thus, the mesenchymal stem population International Application WO 2018/067071 is the ideal starting material for producing the CLiPS of the present invention under GMP conditions.

It is noted in this context that CLMCs transfected with a transgene will maintain their stemness and stem cell characteristics but may show a decrease in the percentage of cells expressing mesenchymal stem cell markers such as CD73, CD90 and CD105 while at the same time may also show an increase in the percentage of cells expressing negative markers such as CD34, CD45 or HLA-DR. See, Yap et al., Malaysian J Pathol 2009; 31(2): 113-120); cf. also Madeira et al, Journal of Biomedicine and Biotechnology. Volume 2010, Article ID 735349, 12 pages. In light of this, it may be possible that a CLiPS of the present invention that has been generated by reprogramming of a CLMC described herein and isolated from the amniotic membrane of the umbilical cord, may be a stem cell population, wherein at least about 81% or more, about 82% or more, at least 83% or more, at least 84% or more, at least about 85% or, about 86% or more, about 87% or more, about 88% or more, about 89% or more, about 90% or more, about 91% or more, about 92% or more, about 93% or more, about 94% or more, about 95% or more, about 96% or more, about 97% or more, about 98% or more about 99% or more cells of the CLiPS population may express each of the following markers: CD73, CD90 and CD105. In addition, such a CLMC derived population of induced pluripotent stem cells of the invention may be a population, wherein at least about 81% or more, about 82% or more, at least 83% or more, at least 84% or more, at least about 85% or, about 86% or more, about 87% or more, about 88% or more, about 89% or more, about 90% or more, about 91% or more, about 92% or more, about 93% or more, about 94% or more, about 95% or more, about 96% or more, about 97% or more, about 98% or more about 99% may lack expression of each of CD34, CD45 and HLA-DR. One preferred example of such CLMC derived population of induced pluripotent stem cells of the invention may be a population, in which at least about 90% or more, about 91% or more, about 92% or more, about 93% or more, about 94% or more, about 95% or more, about 96% or more, about 97% or more, about 98% or more about 99% or more cells of the CLMC population express each of CD73, CD90 and CD105 and lack expression of each of CD34, CD45 and HLA-DR.

Turning again to the generation of an induced pluripotent stem cell (population) of the invention, it is again important to note that such an induced pluripotent stem cell is obtainable by any suitable method that reprograms a stem cell (population) of the amniotic membrane of umbilical cord into such an induced pluripotent stem cell (population). While one method of generating such an induced pluripotent stem cell comprises expressing exogenous nucleic acids encoding the proteins OCT3/4, SOX2, KLF4, LIN28 and L-MYC and the p53-shRNA in a stem cell of the amniotic membrane of the umbilical cord under conditions suitable to reprogram the stem cell, thereby generating the induced pluripotent stem cell, the invention is by no means restricted to CLiPS obtained by this method. Rather, the CLiPS can be obtained by any suitable method as, for example described in the review of Cieslar-Probuda et al “Transdifferentiation and reprogramming Overview of the processes, their similarities and differences” BBA-Molecular Cell Research, Volume 1864, Issue 7, July 2017, Pages 1359-1369. For example, the reprogramming may be performed in the present invention also chemically by using small molecules or biologically by expressing exogenous nucleic acids encoding for reprogramming factors within a cell. Alternatively, the exogenous nucleic acids encoding the proteins OCT3/4, SOX2, KLF4, LIN28, L-MYC and the p53 shRNA may be provided as any suitable nucleic acid for expression. For example, the nucleic acid may be deoxyribonucleic acid (DNA), ribonucleic acid (RNA) comprising messenger RNA (mRNA) and microRNA (miRNA). The exogenous nucleic acids may be transferred as such or the exogenous nucleic acids may be incorporated into one or more vector(s) suitable to be transferred into a cell. In this context, any vector suitable to be transferred into CLSC can be used. An illustrative example for such a vector may be a plasmid. In the present invention, the exogenous nucleic acids may be provided by one, two, three or four vectors suitable to be transferred into a stem cell. In an illustrative example, three vectors may provide the exogenous nucleic acids for reprogramming CLSC into CLiPS, wherein the vectors may be pCXLE-hOCT3/4-shp53-F (Addgene plasmid #27077; Sequence ID No: 12), pCXLE-hSK (Addgene plasmid #27078, Sequence ID No: 13) and pCXLE-hUL (Addgene plasmid #27080; Sequence ID No: 14).

In accordance with the above, any method suitable for transferring exogenous nucleic acids or a protein into the CSLC can be used. In one example, a viral vector may be used to transfer the exogenous nucleic acid into the CSLC. An example for such a viral vector may be a retrovirus, a lentivirus, an inducible lentivirus, a sendai virus or an adeno virus. Alternatively, transfection may be performed to transfer the exogenous nucleic acids into CSLC. In the present invention, transfection may comprise electroporation, microinjection, liposome- and non-liposome-mediated transfection and sonoporation.

In a preferred example, the CLSC may be subjected to electroporation, wherein the electric parameters may be adjusted depending on the type of CLSC being used, as a CLMC may require different electroporation conditions than a CLEC. The electric parameters may comprise the number of electric pulses applied to the stem cell, the duration time of the applied electric pulse(s) and the voltage of the applied electric pulse(s). Each electric parameter may be adjustable to further optimize the electroporation of the present invention. When so doing, each electric parameter may be adjusted independently or in combination with one or more of the other electric parameter(s) (cf. Example 1). In the present invention, any parameter setting suitable for allowing the transfer of exogenous nucleic acid into CLSC may be applied. In one example of the present invention, a CLMC may be subjected to electroporation. In such a case, the electroporation may be carried out with 1 electric pulse which may have a duration time of about 15 milliseconds (ms) to about 25 ms and a voltage of about 1550 V to about 1650 V. Accordingly, in one example, a CLMC may be subjected to electroporation with 1 electric pulse, which may have a duration time of about 20 ms and a voltage of about 1600 V. In addition, it has been found herein that electroporation yielding usable amounts/numbers of CLiPS derived from CLMC depends on the ratio of each vector (plasmid) DNA transfected to the number of CLMC used for the transfection. This ratio is expressed herein by the amount of each vector (plasmid) DNA (in μg) that is used to the number of CLMC (in 1×10⁶ cell) subjected to electroporation. In illustrative examples, the ratio of the amount of vector (plasmid) DNA for each vector to the number of cells may be in the range of 1.5 μg DNA to about 1×10⁶ CLMC to about 2.5 μg DNA to about 1×10⁶ CLMC. Thus, this ratio may be about 2.5 μg DNA to about 1×10⁶ CLMC, about 2.25 μg DNA to about 1×10⁶ CLMC, about 1.8 μg DNA to about 1×10⁶ CLMC, about 1.7 μg DNA to about 1×10⁶ CLMC, about 1.67 μg DNA to about 1×10⁶ CLMC, about 1.6 μg DNA to about 1×10⁶ CLMC, or about 1.5 μg DNA to about 1×10⁶ CLMC (cf. Table 1 showing that using a ratio of the amount of vector (plasmid) DNA for each plasmid to the number of cells of about 1.67 μg DNA to about 1×10⁶ CLMC yielded an effective transformation yield). Thus, in one embodiment of generating CLiPS derived from CLMC, it is preferred that each of the vectors is used in the same amount in the electroporation of the CLMC. Also a CLEC may be subjected to electroporation to yield CLiPS of the invention. In case of CLiPS derived from CLEC, the electroporation may be carried out with 2 electric pulses, which may each have a duration time of about 25 ms to about 35 ms and a voltage of about 1300 V to about 1400 V each. Accordingly, in one example, a CLEC may be subjected to electroporation with 2 electric pulses, which may have a duration time of about 30 ms and a voltage of about 1350 V each. As for CLMC, it has also been found for CLiPS derived from CLEC that electroporation yielding usable amounts/numbers of CLiPS derived from CLEC depends on the ratio of the amount of each plasmid DNA transfected to the number of CLEC used for the transfection. Also this ratio is expressed herein by the amount of vector (plasmid) DNA (in μg) that is used for transfection to the number of CLEC (in 1×10⁶ cells) which is to be transfected. In illustrative examples, the ratio of the amount of vector (plasmid) DNA to the number of cells may be in the range of about 1.5 μg DNA to about 1×10⁶ CLEC to about 2.5 μg DNA to about 1×10⁶ CLEC. Thus, the ratio may be about 1.5 μg DNA to about 1×10⁶ CLEC, about 1.6 μg DNA to about 1×10⁶ CLEC, about 1.67 μg DNA to about 1×10⁶ CLEC, about 1.7 μg DNA to about 1×10⁶ CLEC, about 1.8 μg DNA to about 1×10⁶ CLEC, about 1.9 μg DNA to about 1×10⁶ CLEC, about 2.0 μg DNA to about 1×10⁶ CLEC, or about 2.5 μg DNA to about 1×10⁶ CLEC (cf. Table 1 showing that using a ratio of the amount of plasmid DNA for each vector to the number of cells of about 1.67 μg DNA to about 1×10⁶ CLEC provided an effective transformation yield). Thus, in one embodiment of generating CliPS derived from CLEC, it is preferred that each of the vectors is used in the same amount in the electroporation of the CLEC. The electroporation of both CLEC and CLMC may be performed in the method of the invention in a uniform electrical field. Thereby, critical consequences of the electroporation such as pH change, ion formation or heat generation may be minimized. The uniform electric field may be generated by maximizing the gap between the electrodes while minimizing the surface area of each electrode. An illustrative example for a system providing such a uniform electric field is the Neon™ Transfection System of ThermoFisher Scientific. Another example of a suitable commercial transfection system is The Gene Pulser MXcell electroporation system, available from Bio-Rad. As a final remark, the transfection can be carried using any suitable electroporation buffer. In case a commercial transfection system such as the Neon™ Transfection System is used, the respective electroporation buffer provided by the manufacturer of the transfection system is typically used for electroporation.

After transfection, the stem cells may be transferred into a medium suitable for cell recovery and cell cultivation. In the present invention, any cell culture medium suitable for cell recovery and/or proliferation can be used. Illustrative examples for such a suitable cell culture medium may be commonly used media for cultivation (propagation) of human induced pluripotent stem cell such as mTeSR1, StemMACS™ iPS-Brew XF, TeSR™-E8, mTeSR™ Plus, TeSR™2, mTeSR™1. It is also possible to use for the cell recovery cultivation any medium that capable of supporting proliferation (without differentiation)/healthy growth of CLEC or CLMC. Examples of suitable media for this cultivation of CLEC are, for example, described in US patent application 2006/0078993 and include EpiLife medium, Medium 171, MEGM-Mammary Epithelial Cell Medium or mixtures of such media such as the medium PTT-e3 (that has been used herein for the generation of CLiPS derived from CLEC and that is described herein in detail below). Examples of suitable media for this cultivation of CLMC are, for instance, described in US patent applications 2006/0078993 and 2018/127721 as well as in International Patent Application WO2007/046775 and include DMEM/10% FBS, DMEM:F12 culture medium (a 1:1 mixture of DMEM and Ham's F-12 medium), or a media such as PPT-6 (a culture medium comprising DMEM, F12-medium, Medium 171 and FBS, see US application 2018/127721) or PTT4 (wherein the latter has been used in the Example Section herein for the generation of CLiPS derived from CLMC). It is also possible to use for this cell recovery cultivation mixtures of these media (for example a mixture of mTeSR1 with the medium PTTe-3 or the medium PTT-4). The medium suitable for cell recovery of transfected CLEC or CLMC as described herein may further contain growth factors, which may stimulate cellular growth and proliferation. The growth factors may be added to the cell culture medium as such. In addition, the recovery medium may contain serum such as, for example, fetal bovine serum (FBS). Thus, the medium suitable for cell recovery after transfection may be a serum-free or a serum-containing medium.

In line with the above disclosure, the composition of the medium suitable for cell recovery may differ, depending on the CLSC being used.

For example, the medium suitable for the recovery of a transfected CLMC may consist of a (chemically) defined medium and FBS. Accordingly, the medium suitable for the recovery of a transfected CLMC may consist of about 80% (v/v), about 85% (v/v), about 90% (v/v) or about 95% (v/v) chemically defined medium and about 20% (v/v), about 15% (v/v), about 10% (v/v) or about 5% (v/v) FBS, respectively. In a preferred example, CLMC are cultivated in medium PTT-4 for cell recovery after transfection, wherein medium PTT-4, as described in International Patent Application WO2007/046775, consists of 90% (v/v) CMRL-1066 and 10% (v/v) FBS. A medium suitable for the recovery of a transfected CLEC may be a serum-free medium, wherein the medium may contain cytokines and growth factors.

Also the medium suitable for the recovery of a transfected CLEC may be a defined medium. Such a recovery medium may comprise Mammary Epithelial Basal Medium MCDB 170, EpiLife medium, DMEM (Dulbecco's modified eagle medium), F12 (Ham's F12 Medium) and FBS (Fetal Bovine Serum).

In illustrative examples, such a medium comprises Mammary Epithelial Basal Medium MCDB 170 in a final concentration of about 10 to about 30% (v/v), EpiLife medium in a final concentration of about 20 to about 40% (v/v), F12 in a final concentration of about 5 to about 15% (v/v), DMEM in a final concentration of about 30 to about 45% (v/v) and FBS in a final concentration of about 0.1 to 2% (v/v). One such medium may comprise Mammary Epithelial Basal Medium MCDB 170 in a final concentration of about 15 to about 25% (v/v), EpiLife medium in a final concentration of about 25 to about 35% (v/v), F12 in a final concentration of about 7.5 to about 13% (v/v), DMEM in a final concentration of about 35 to about 40% (v/v) and FBS in a final concentration of about 0.5 to 1.5% (v/v). Another such medium may comprise Mammary Epithelial Basal Medium MCDB 170 in a final concentration of about 20% (v/v), EpiLife medium in a final concentration of about 30% (v/v), F12 in a final concentration of about 12.5 (v/v), DMEM in a final concentration of about 37.5% (v/v) and FBS in a final concentration of about 1.0% (v/v). The value of “% (v/v)” as used herein refers to the volume of the individual component relative to the final volume of the culture medium. This means, if DMEM is, for example, present in the culture medium a final concentration of about 35 to about 40% (v/v), 1 liter of culture medium contains about 350 ml to 400 ml DMEM. In one embodiment, the medium suitable for the recovery of a transfected CLEC cell is obtained by mixing to obtain a final volume of 1000 ml culture medium:
200 ml Mammary Epithelial Basal Medium MCDB 170,
300 ml EpiLife medium,
250 ml DMEM,
250 ml DMEM/F12, and
1% Fetal Bovine Serum.

The growth factors in the medium suitable for the recovery of a transfected CLEC may an insulin like growth factor (IGF) such as IGF-1 or IGF-2, an epidermal growth factor (EGF) such as HB-EGF or EPR, a transforming growth factor (TGF) such as TGF-α or TGF-β 1, an activin, a bone morphogenic protein (BMP), a platelet derived growth factor (PDGF), transferrin and insulin. In one example, CLEC are cultivated in medium PTTe-3 for cell recovery after transfection, wherein medium PTTe-3 contains human epidermal growth factor (EGF), one or more transforming Growth Factors such as TGF-alpha and/or TGF-beta (TGF-beta 1, TGF-beta 2 and/or TGF-beta 3), or insulin.

In accordance with the above, the medium suitable for the recovery of a transfected CLEC may comprise human epidermal growth factor (EGF) in a final concentration of about 1 to about 15 ng/ml. The recovery medium may also comprise insulin in a final concentration of about 1 to about 7.5 μg/ml. This recovery medium may further comprise at least one of the following supplements: adenine, hydrocortisone, and 3,3′,5-Triiodo-L-thyronine sodium salt (T3). In one embodiment the medium comprises all three of adenine, hydrocortisone, and 3,3′,5-Triiodo-L-thyronine sodium salt (T3). In this case, the medium may comprise adenine in a final concentration of about 0.05 to about 0.1 mM adenine, hydrocortisone in a final concentration of about 0.1 to 0.5 μM hydrocortisone and 3,3′,5-Triiodo-L-thyronine sodium salt (T3) in a final concentration of about 0.1 to about 5 ng/ml. The recovery medium may comprise one of more transforming growth factors (TGF), for example transforming growth factor beta 1 (TGF-beta 1) and/or transforming growth factor alpha (TGF-alpha. In such a medium, TGF-beta 1 may be present in a final concentration of about 0.1 to about 5 ng/ml and TGF-alpha may be present in a final concentration of about 1.0 to about 10 ng/ml. In addition, the medium of recovery of CLEC may comprises Cholera Toxin from Vibrio cholerae (which is commercially available, for example, from Sigma Aldrich under catalogue number C8052. If cholera toxin from Vibrio cholerae is used, it may be present in a final concentration of about 1×10⁻¹¹ M to about 1×10⁻¹⁰ M.

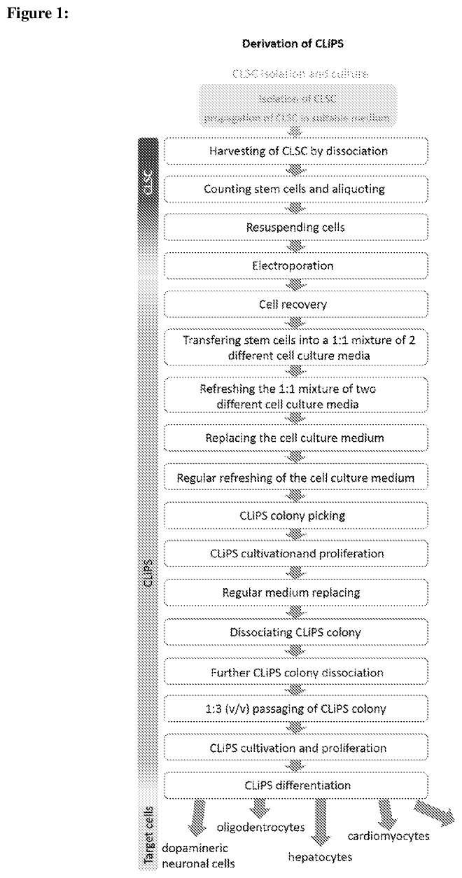

By “DMEM” is meant Dulbecco's modified eagle medium which was developed in 1969 and is a modification of basal medium eagle (BME) (cf. FIG. 1 showing the data sheet of DMEM available from Lonza). The original DMEM formula contains 1000 mg/L of glucose and was first reported for culturing embryonic mouse cells. DMEM has since then become a standard medium for cell culture that is commercially available from various sources such as ThermoFisher Scientific (catalogue number 11965-084), Sigma Aldrich (catalogue number D5546) or Lonza, to name only a few suppliers. Thus, any commercially available DMEM can be used in the present invention. In preferred embodiments, the DMEM used herein is the DMEM medium available from Lonza under catalog number 12-604F. This medium is DMEM supplemented with 4.5 g/L glucose and L-glutamine). In another preferred embodiment the DMEM used herein is the DMEM medium of Sigma Aldrich catalogue number D5546 that contains 1000 mg/L glucose, and sodium bicarbonate but is without L-glutamine.

By “F12” medium is meant Ham's F12 medium. This medium is also a standard cell culture medium and is a nutrient mixture initially designed to cultivate a wide variety of mammalian and hybridoma cells when used with serum in combination with hormones and transferrin. Any commercially available Ham's F12 medium (for example, from ThermoFisher Scientific (catalogue number 11765-054), Sigma Aldrich (catalogue number N4888) or Lonza, to name only a few suppliers) can be used in the present invention. In preferred embodiments, Ham's F12 medium from Lonza is used. By “DMEM/F12” or “DMEM:F12” is meant a 1:1 mixture of DMEM with Ham's F12 culture medium. Also DMEM/F12 (1:1) medium is a widely used basal medium for supporting the growth of many different mammalian cells and is commercially available from various supplier such as ThermoFisher Scientific (catalogue number 11330057), Sigma Aldrich (catalogue number D6421) or Lonza. Any commercially available DMEM:F12 medium can be used in the present invention. In preferred embodiments, the DMEM:F12 medium used herein is the DMEM/F12 (1:1) medium available from Lonza under catalog number 12-719F (which is DMEM:F12 with L-glutamine, 15 mM HEPES, and 3.151 g/L glucose).

By “M171” is meant culture medium 171, which has been developed as basal medium for the culture of for the growth of normal human mammary epithelial cells. Also this basal medium is widely used and is commercially available from supplier such as ThermoFisher Scientific or Life Technologies Corporation (catalogue number M171500), for example. Any commercially available M171 medium can be used in the present invention. In preferred embodiments, the M171 medium used herein is the M171 medium available from Life Technologies Corporation under catalogue number M171500.

By “Mammary Epithelial Basal Medium MCDB 170” is meant a basal nutrient medium that is used for the growth of mammary epithelial cells and that is commercially available in powder form, for example, from United States Biological, Salem Massachusetts USA under catalogue number M2162 or from Bio-Connect B. V., Huissen, The Netherlands, under catalogue number (MBS652676_101)

By EpiLife medium is meant a HEPES and bicarbonate buffered liquid medium that is prepared without calcium chloride and that is commonly used for the long-term, serum-free culture of human epidermal keratinocytes and human corneal epithelial cells and is designed for use in an incubator with an atmosphere of 5% CO2 and 95% air. Available from ThermoFisher Scientific, catalogue number MEPICF500 or from Sigma Aldrich under Product Code E 0151.

By “CMRL medium” is meant the medium that was originally developed by Connaught Medical Research Laboratories for the growth of Earle's ‘L’ cells under serum-free conditions. CMRL medium is known to be also especially useful for cloning monkey kidney cells and for growth of other mammalian cell lines when supplemented with horse or calf serum. CMRL medium is commercially available, for example, from ThermoFisher Scientific (catalogue number 11530037)

By “FBS” is meant fetal bovine serum (that is also referred to as “fetal calf serum”), i.e. the blood fraction that remains after the natural coagulation of blood, followed by centrifugation to remove any remaining red blood cells. Fetal bovine serum is the most widely used serum-supplement for in vitro cell culture of eukaryotic cells because it has a very low level of antibodies and contains more growth factors, allowing for versatility in many different cell culture applications. The FBS is preferably obtained from a member of the International Serum Industry Association (ISIA) whose primary focus is the safety and safe use of serum and animal derived products through proper origin traceability, truth in labeling, and appropriate standardization and oversight. Suppliers of FBS that are ISIA members include Abattoir Basics Company, Animal Technologies Inc., Biomin Biotechnologia LTDA, GE Healthcare, Gibco by Thermo Fisher Scientific and Life Science Production, to mention only a few. In currently preferred embodiments, the 1-BS is obtained from GE Healthcare under catalogue number A15-151.

The medium suitable for cell recovery may also contain a compound, which may suppress an inflammatory response and/or may also enhance cell survival and proliferation after transfection. An illustrative example for such a compound may be a glucocorticoid. Glucocorticoids are steroid hormones, which are able to up-regulate the expression of anti-inflammatory proteins in the nucleus and repress the expression of pro-inflammatory proteins in the cytosol. The glucocorticoid used herein may be prednisolone, methylprednisolone, dexamethasone, betamethasone, corticosterone or hydrocortisone, to name only a few illustrative examples of suitable glucocorticoids. It is also possible to use two or more such of glucocorticoids together, for example, a mixture of corticosterone and hydrocortisone. The glucocorticoid can be used in any suitable concentration, for example, in a concentration of about 0.1 μM to about 2.5 μM or to about 5 μM. In one illustrative example, the glucocorticoid in the medium suitable for the recovery of transfected CLSC may be hydrocortisone used in a concentration of about 0.1 μM to about 2.5 μM. In one example, the hydrocortisone concentration in the medium suitable for the recovery of transfected CLSC is about 0.5 μM to about 2 μM. In one such illustrative example, the hydrocortisone concentration is about 1 μM.

The recovery of the transfected CLSC may be carried out in a cell culture device such as a cell culture vessel. The cell culture vessel may be, but is not limited to, a cultivation flask, a petri dish, a roller bottle and a multiwall plate. Further, the cell culture vessel may be coated to provide a layer, which may facilitate the cell growth by supplying the cells with metabolites. The coating of the cell culture vessel may be serum-derived or serum-free. An example for a serum-derived coating may be a coating with gelatinous proteins from the basement membrane-like matrix such as Matrigel. A serum-free coating of the cell culture vessel may instead be characterized by being animal and xeno-free thus allowing a cell cultivation under cGMP conditions. An example for a serum-free coating of the cell culture vessel may be a coating with recombinant proteins or parts thereof such as, for example, a coating with a extracellular matrix protein such as collagen, fibronectin, elastin, laminin, including, for instance, the laminin-511 E8 fragment, or laminin 521, vitronectin, for example, in the form of commercially available citronectin XF™, CELLstart or the Synthemax™ vitronectin substrate. In one example of the present invention transfected CLEC may be preferably cultivated in a cell culture vessel with a serum-derived coating, whereas CLMC may be preferably cultivated in a cell culture vessel with a serum-free coating.

The medium suitable for the recovery of the transfected CLSC may be replaced with another cell culture medium after a suitable period of time. The suitable period of time may, for example, be about 1, about 2 or about 3 days after transfection. Thus, in one example, the medium replacement may be carried out about 2 days after transfection. Another cell culture medium used for the medium replacement may also be a mixture of different cell culture media. In the present invention, any cell culture medium or cell culture medium mixture suitable for yielding of iPS can be used. Furthermore, the suitable cell culture medium or cell culture media mixture may contain a compound, which may suppress inflammatory response and enhance cell survival. In the present invention the medium suitable for cell recovery after transfection may be replaced with a mixture of two different cell culture media after a suitable period of time to ensure a proper supply of nutrients and a suitable blend of growth factors to the cells as they transition from their native state into a more pluripotent state when undergoing somatic reprogramming. Accordingly, the cell culture media mixture of the present invention may consist of the medium suitable for cell recovery, which may contain hydrocortisone, and a second cell culture medium. In a preferred example, the two different cell culture media are mixed in a ratio of about 1:1 (v/v), wherein the mixture may be prepared by contacting 1 volume medium suitable for cell recovery to 1 volume second cell culture medium. In a another preferred example, the two different cell culture media are mixed in a ratio of about 1:2 (v/v) or 2:1, wherein the mixture may be prepared by contacting 1 volume medium suitable for cell recovery to 2 volumes second cell culture medium (or 2 volumes medium suitable for cell recovery to 1 volumes second cell culture medium) The second cell culture medium used for generating the cell culture mixture may be any cell culture medium suitable to enhance or maintain iPS proliferation (such medium is also termed “maintenance medium” herein). Using a mixture such a 1:1 mixture of the medium used for cell recovery and the maintenance medium provides the advantage of allowing the CLiPS cells to transition gradually from their cognate culture medium to the ES/iPSC medium, instead of a sudden switch that might compromise their viability. Without wishing to be bound by theory, it is assumes that about two days after transfection, some successfully transfected cord lining stem cells will start to acquire pluripotent stem cell characteristics and concomitantly, acquire nutrient requirements of PSCs. Illustrative examples for such a suitable cell culture medium include, but are not limited to, commercially maintenance media such as mTeSR1, StemMACS™ iPS-Brew XF, TeSR™-E8, mTeSR™ Plus, TeSR™2 or mTeSR™1, Corning® NutriStem® hPSC XF Medium, Essential 8 Medium (ThermoFisher Scientific), StemFlex (ThermoFisher Scientific), StemFit Basic02 (Ajinomoto Co. Inc), or PluriSTEM (Merck Millipore). The medium mTeSR™1, since being manufactured under GMP conditions may be preferably used, if the iPS colonies are cultivated under animal- and xeno-free GMP conditions. Thus, in one preferred example, mTeSR1 may be the second cell culture medium used for generating the cell culture mixture. In the present invention, the 1:1 (v/v) cell culture media mixture may be replaced with the same mixture of cell culture media within a suitable period of time. This suitable period of time may be about 3, about 4, about 5 or about 6 days after transfection. Thus, in one example, the 1:1 (v/v) cell culture media mixture may be replaced with the same mixture 4 days after transfection. After a suitable period of time, the 1:1 (v/v) cell culture media mixture may be further replaced with the second cell culture medium used for generating the cell culture mixture only. In this context, a suitable period of time may be about 4, about 5, about 6 or about 7 days after transfection. In one example, the 1:1 (v/v) cell culture media mixture may be replaced with the second cell culture medium 6 days after transfection. In a preferred example, the 1:1 (v/v) cell culture media mixture may be replaced with mTeSR1 and mTeSR™1, respectively, 6 days after transfection. The regular cell culture media changes and replacements may contribute to an increase of surviving CLiPS. Thus, CLiPS colonies may grow and proliferate.

After changing the cell culture media mixture to one cell culture medium, the CLiPS may be further cultivated. For this purpose, the cell culture medium may also be replaced regularly with the same medium to ensure a proper supply of nutrients and a suitable blend of growth factors to the cells. For example, the cell culture medium may be replaced daily or every second day, every third day or every fourth day. In one example of the present invention, the cell culture medium may be replaced every second day. Consequently, CLiPS colonies may further grow and proliferate.

CLiPS colonies may become visible to the naked eye about 10, 11, 12, 13, 14, 15, or 16 days after transfection (cf., Example 2). When reaching a suitable size, CLiPS may be selected and transferred to another coated culture vessel for further cultivation and proliferation. In this context, a suitable colony size may comprise a length of about 0.1 mm to about 2 mm in diameter. In one example of the present invention, the CLiPS colony may be selected when reaching a length of about 0.5 mm to about 1.5 mm in diameter, wherein the CLiPS colonies may reach this size about 20 days after transfection. To transfer a CLiPS colony with a suitable size to another cultivation vessel, the CLiPS colony may be picked. This may be carried out manually, if wanted. To facilitate the colony picking, a device allowing an enlarged view of the colonies may be used. Examples for such a device may be a magnifier or a microscope. In the present invention, the CLiPS may be selected and picked under bright field microscopy. Turning to the cell culture vessel, the picked CLiPS colonies may be transferred to another cell culture vessel, wherein the coating of the cell culture vessel may vary from the coating of the cell culture vessel used for the recovery of the transfected CLSC or it may be the same. In a preferred example, the coating of the culture vessel is the same, since CLMC-derived CLiPS thus far cultivated under cGMP suitable conditions may be maintained animal- and xeno-free, thereby preserving cGMP conditions. Consequently, a CLMC-derived CLiPS colony, for example, may be transferred in a cell culture vessel coated with a serum-free substance such as the laminin-511 E8 fragment for further cultivation (cf., Example 3). Alternatively, a CLEC- and/or CLMC-derived CLiPS colony, for example, may be transferred in a cell culture vessel coated with a serum-derived substance such as Matrigel for further cultivation. The cell culture medium may preferably be the same as used before the colony picking. In the examples of the present invention, the cell culture medium may be also replaced regularly after colony picking. For example, medium may be replaced daily, every second day or every third day. In a preferred example of the present invention, the cell culture medium may be replaced daily after colony picking.

When reaching a suitable confluence, the CLiPS colonies or a cell population formed from the colonies are typically detached from the coated cell culture vessel and transferred to a larger cell culture vessel for further cultivation under the same cultivation conditions used directly after the colony picking. A suitable confluence may be at least about 40%, at least about 45%, at least about 50%, at least about 55%, at least about 60% and at least about 65% confluence. It is noted in this context that the term “cell population” when used in relation to the propagation of CLiPS forming colonies is more suitable as the CLiPS cells do not take on a colony-like appearance when they reach a confluence of about 70% to about 80%. For detaching the CLiPS colonies or a cell population formed from the colonies from the coated cell culture vessel, any dissociation agent suitable to disrupt cell adhesion or hydrolyze peptide bonds can be used. An example for such a suitable dissociation agent may be a solution containing a chelating agent such as ethylenediaminetetraacetic acid (EDTA) or a solution containing an enzyme such as trypsin or dispase (see the experimental section of the present application, in which dispase has been used to detach a CLiPS colony from the coated cell culture vessel). The cell culture medium may also be replaced regularly, for example, daily, every second day or every third day. In a preferred example of the present invention, the cell culture medium may be replaced daily. This way, the CLiPS may further grow and proliferate.

In the present invention, a CLiPS colony or a cell population formed from a colony may be passaged when reaching a suitable size. The suitable size may correspond to about 50%, about 55%, about 60%, about 65%, about 70%, about 75%, about 80%, about 85%, about 90% and about 95% confluence. In an example of the present invention, the CLiPS colony or the cell population formed therefrom may be passaged when the culture reaches about 60-90% confluence. Thus, in a preferred example, the CLiPS colony or the cell population formed therefrom may be passaged when reaching about 70-80% confluence. For passaging, CLiPS may be passaged in a suitable ratio, wherein one volume CLiPS may be contacted with multiple volumes of cell culture medium. In the present invention, CLiPS may be passaged in a ratio of about 1:3 (v/v), or about 1:4 (v/v), or about 1:5 (v/v) or about 1:6 (v/v), wherein the passaging may be performed by dividing 1 volume dissociated CLiPS into about 2, or about 3, or about 4 or about 5 volumes of dissociated CLiPS, respectively. In a preferred example, CLiPS may be passaged in a ratio of about 1:3 (v/v). To allow passaging of cultivated CLiPS of the present invention, again any enzyme suitable to detach the cells from the culture vessel can be used. For example, dispase may be used for this purpose. Further, any chemical suitable to remove cell-to-cell adhesion can be used for CLiPS passaging in the context of the present invention, wherein the concentration of the chemical may be suitable to remove cell-to-cell adhesion without harming the cells. An illustrative example for such a chemical may be EDTA. Since EDTA may kill cells at higher concentrations, a suitable EDTA concentration of the present invention may be about 0.5 mM. In the present invention, the cell culture medium used for passaging may be supplemented with a substance suitable for enhancing the survival of the CLiPS when dissociated. For this purpose, any substance suitable for enhancing the survival of the CLiPS when dissociated may be used. An example of such a suitable substance may be an inhibitor of a signaling pathway such as the rho-associated protein kinase (ROCK) signalling pathway. Thus, the RHO/ROCK pathway inhibitor Y-27632 may be an illustrative example for a substance suitable for enhancing the survival of dissociated CLiPS. Alternatively, a defined supplement for single-cell cloning of human iPS cells such as CloneR™ (available from StemCell Technologies) may be also be used for enhancing the survival of the dissociated cells. In the present invention, the passaged CLiPS may be cultivated in a medium supplemented with the substance suitable for enhancing the survival of the dissociated CLiPS for a suitable period of time before getting differentiated into a target cell.

By cultivating CLiPS after passaging, a master cell bank containing (primary) isolated CLiPS can be obtained. For generating a master cell bank of CLiPs, CLiPS cells obtained by the process as described herein can be seeded in a cultivation vessel such as a cell culture plate. CLiPS can, for this purpose, be suspended and cultured in any suitable medium, typically a maintenance medium for iPS cells such as commercially media mentioned above such as mTeSR1, StemMACS™ iPS-Brew XF, TeSR™ E8, mTeSR™ Plus, TeSR™2 or mTeSR™1, Corning® NutriStem® hPSC XF Medium, Essential 8 Medium (ThermoFisher Scientific), StemFlex (ThermoFisher Scientific), StemFit Basic02 (Ajinomoto Co. Inc), or PluriSTEM (Merck Millipore). Both CLiPS derived from CLMC and CliPS derived from CLEC can be cultivated in such a iPS maintenance medium. For subculturing, the CLiPS cells (of both CLMC- and CLEC derived CLiPS) can be seeded at any suitable concentration, for example, or a concentration of about 0.5×10⁶ cells/ml to about 5.0×10⁶ cells/ml. In one example, the cells are suspended for subcultivation at a concentration of about 1.0×10⁶ cells/ml. The subculturing can be carried by cultivation either in simple culture flasks but also, for example, in a multilayer system such as CellSTACK (Corning, N.Y., USA) or Cell Factory (Nunc, part of Thermo Fisher Scientific Inc., Waltham, Mass., USA) that can be stacked in incubators. Alternatively, the subculturing can also be carried out in a closed self-contained system such as a bioreactor. Different designs of bioreactors are known to the person skilled in the art, for example, parallel-plate, hollow-fiber, or micro-fluidic bioreactors. See, for example, Sensebe et al. “Production of mesenchymal stromal/stem cells according to good manufacturing practices: a review”, supra. An illustrative example of a commercially hollow-fiber bioreactor is the Quantum® Cell Expansion System (Terumo BCT, Inc), that has, for example, been used for the expansion of bone marrow mesenchymal stem cells for clinical trials (cf., Hanley et al, Efficient Manufacturing of Therapeutic Mesenchymal Stromal Cells Using the Quantum Cell Expansion System, Cytotherapy. 2014 August; 16(8): 1048-1058) and for the expansion of the highly pure cord ling mesenchymal stem cell population described in International Patent Application WO 2018/067071. Another example of a commercially available bioreactors that can be used for the subculturing of the CLiPS population of the present invention is the Xuri Cell Expansion System available from GE Healthcare. The cultivation of the CLiPS population in an automated system such as the Quantum® Cell Expansion System is of particular benefit if a working cell bank for therapeutic application is to be produced under GMP conditions and a high number of cells is wanted. Also for the subcultivation, CLiPS can be cultured till a suitable amount of cells have grown. In illustrative examples CLiPS are subcultivated till the CLiPS reach about 70% to about 80% confluency. The isolation/cultivation of the population of CLiPS can be carried out under standard condition for the cultivation of mammalian cells. Once a desired/suitable number of CLiPS have been obtained from the subculture, the cells are harvested by removing them from the cultivation vessel used for the subcultivation. The CLiPS harvesting is typically carried out by enzymatic treatment. The isolated CLiPS are subsequently collected and are either be directly used or preserved for further use. Typically, preserving is carried out by cryo-preservation. The term “cryo-preservation” is used herein in its regular meaning to describe a process where here CLiPS are preserved by cooling to low sub-zero temperatures, such as (typically) −80° C. or −196° C. (the boiling point of liquid nitrogen). Cryopreservation can be carried out as known to the person skilled in the art and can include the use of cryo-protectors such as dimethylsulfoxide (DMSO) or glycerol, which slow down the formation of ice-crystals in the CLiPS cells.

The present invention is also directed to CLiPS obtainable by the method as described herein and to CLiPS obtained by the method as described herein. CLiPS obtainable/obtained by the present invention may grow and proliferate robustly (cf. Example 2 and Example 3). Thereby, CLiPS cultivation may be more efficient in comparison to a cultivation of iPS derived from, for example, the bone marrow stroma, fat tissue, the dermis or the Wharton's jelly. Analysis of CLiPS functionality reveals expression of human embryonic stem cell markers indicating self-renewal properties and a normal karyotype (cf. Example 4 and Example 5). Further, CLiPS are capable to differentiate into multiple cell types (functional target cells) in vitro and in vivo indicating pluripotency (cf. Example 6). Therefore, CLiPS are highly suitable for medical and therapeutic applications. Consequently, the present invention is also directed to a pharmaceutical composition comprising an iPS obtainable/obtained by the method described herein.

The present invention is further directed to a method of differentiating a CLiPS into a target cell under conditions suitable for differentiation. Examples of a suitable target cell include, but are by no means limited to, a neuronal cell, dopaminergic neuronal cell, an oligodentrocyte, an astrocyte, a cortical neuron, a hepatocyte, a cartilage cell, a muscle cell, a bone cell, a dental cell, a hair follicle cell, an inner ear hair cell, a skin cell, a melanocyte, a cardiomyocyte, a hematopoietic progenitor cell, a blood cell, an immune cell, a T- or B-lymphocyte, a microglia, a natural killer cell or a motor neuron, to mention only a few. To facilitate the directed differentiation into a target cell, the CLiPS may be exposed to a priming substance, typically under conditions that are known to the skilled artesian from the differentiation of iPS derived from other sources into the target cell. The exposure may be carried out under suitable conditions, which may comprise a cultivation in a cell culture vessel filled with a cell culture medium suitable for priming the CLiPS differentiation and for subsequent cultivation. In the present invention any cell culture medium suitable for priming, proliferating and differentiating iPS can be used, wherein the medium composition and thus the method of differentiation may depend on the target cell and may be taken from known protocols for the differentiation of iPS into the desired target cell (see in this respect, the reviews of Hirschi et al “Induced Pluripotent Stem Cells for Regenerative Medicine” Annu Rev Biomed Eng. 2014 Jul. 11; 16: 277-294) or Shi et al “Induced pluripotent stem cell technology: a decade of progress” Nat Rev Drug Discov. 2017 February; 16(2): 115-130). For example, CLiPS may be cultivated in a medium adapted for proliferation and differentiation of the CLiPS into a dopaminergic neuronal cell. In such a case, the medium may be a Neurobasal medium supplemented with a growth factor such as B-27 minus vitamin A, transforming growth factor 3-β (TGFβ3), a glial cell line-derived neurotrophic factor (GDNF), a brain-derived neurotrophic factor (BDNF), ascorbic acid, dibutyl cAMP, an inhibitor for glycogen synthase kinase 3 such as CHIR99021 and a γ-secretase inhibitor such as (2S)—N-[(3,5-Difluorophenyl)acetyl]-L-alanyl-2-phenyl]glycine 1,1-dimethylethyl ester (DAPT), which induces neuronal differentiation. An illustrative example for such a medium is NB27. A CLiPS differentiation into a dopaminergic neuronal cell is exemplary shown in Example 7. As another example, CLiPS may be cultivated in a medium adapted for proliferation and differentiation of the CLiPS into a hepatocyte. In this case, the medium may be a protein, lipid and growth factor-free medium supplemented with a compound inducing differentiation into a mesoendodermal fate. RPMI 1640-B27 supplemented with Activin A may be an illustrative example for a suitable medium for CLiPS differentiation into a hepatocyte. A CLiPS differentiation into a hepatocyte is exemplary shown in Example 8. As another illustrative example, CLiPS may be cultivated in a medium adapted for proliferation and differentiation of the CLiPS into a cardiomyocyte. In such a case, the medium may be a protein, lipid and growth factor-free medium supplemented with an inhibitor for glycogen synthase kinase 3 such as CHIR99021. RPMI/2%-B27 minus insulin may be an example for a suitable medium for CLiPS differentiation into a hepatocyte. A CLiPS differentiation into a cardiomyocyte is exemplary shown in Example 9. As yet a further illustrative example, CLiPS may be differentiated into an oligodendrocyte using a chemically defined, growth factor-rich medium allowing a differentiation into paired box 6-positive (PAX6+) neural stem cells, which then give rise to oligodendrocyte transcription factor positive (OLIG2+) progenitors (cf. Example 10). In this context, it may be noted that the differentiation of CLiPS into target cells may also be carried out under conditions suitable for cGMP production.

The present invention also includes a pharmaceutical composition comprising a differentiated CLiPS obtained by the method as described herein. The analysis of the immunogenicity of CLiPS and their neural derivates revealed reduced immunogenicity (Example 11). An example for a pharmaceutical composition comprising differentiated CLiPS is an injection solution or any kind of graft suitable for implanting the differentiated CLiPS. In one example, such a graft may comprise differentiated CLiPS-derived multilayered tissue such as an organ or parts thereof. In one example, the graft suitable for implanting the differentiated CLiPS may comprise an implantable matrix coated with differentiated CLiPS. The pharmaceutical composition may be formulated/adapted for parenteral application. In such case, the parenteral application may comprise a sterile preparation intended for injection, infusion or implantation in the human or animal body. Transplantation of CLiPS-derived dopaminergic neurons in fully immunocompetent mice and rat Parkinson's Disease models exhibited functional engraftment and even significant restoration of dopamine reuptake function (cf. Example 12 and Example 13).

The present invention further includes a method of treating a congenital or acquired degenerative disorder in a subject, wherein the subject may be selected from the group comprising a mouse, a rat, a rabbit, a pig, a dog, a cat, a non-human primate or a human. In a preferred example, the subject is human. In this context, treating may comprise administering to a subject a target cell differentiated from CLiPS by the method as described herein. The disease may any known disease which has been considered to be treated by means of cell-based therapy, see in this context, for example, Shi et al “Induced pluripotent stem cell technology: a decade of progress” supra. The congenital or acquired degenerative disorder may have different origins. For example, such a congenital or acquired degenerative disorder may be a neural disorder such as, for example, Parkinson's disease, Alzheimer's disease, Huntington's disease, Amyotrophic lateral sclerosis (ALS), Spinocerebellar ataxia (SCA) and batten disease. Examples for a hepatic degenerative disorder may be inter alia liver failure, liver cirrhosis and viral hepatitis. The congenital or acquired degenerative disorder may also be a cardiac disorder, inter alia comprising acute Danon disease, short-QT syndrome, Brugada syndrome, myocardial infarction, Jervell and Lange-Nielsen syndrome. The disorder may also be an auto-immune disease such as multiple sclerosis.

The present invention is also directed to extracellular membranous vesicles that may be produced by CLiPS or the differentiated derivatives of CLiPS. Such vesicles may include but not exclusively, vesicles ranging from 30 to 150 nanometres (nm) in diameter, also known as exosomes. Originally thought to be primarily responsible for excretory functions, exosomes are now known to be involved in various important biological processes such as cell-cell communication, cellular senescence, proliferation, and differentiation, tissue homeostasis, tissue repair and regeneration, antigen presentation and immune modulation (see, for example, Pegtel, D. M. and S. J. Gould, Exosomes. Annu Rev Biochem, 2019. 88: p. 487-514 or Kalluri, R. and V. S. LeBleu, The biology, function, and biomedical applications of exosomes. Science, 2020. 367(6478). Exosomes have been implicated in a broad range of diseases including cancers (see, for example, Visan, K. S., R. J. Lobb, and A. Moller, The role of exosomes in the promotion of epithelial-to-mesenchymal transition and metastasis. Front Biosci (Landmark Ed), 2020. 25: p. 1022-1057, or Zhang, L. and D. Yu, Exosomes in cancer development, metastasis, and immunity. Biochim Biophys Acta Rev Cancer, 2019. 1871(2): p. 455-468) osteoarthritis (Asghar, S., et al., Exosomes in intercellular communication and implications for osteoarthritis. Rheumatology (Oxford), 2020. 59(1): p. 57-68), diseases of the central nervous system such as such as stroke, Alzheimer's disease (AD), Parkinson's disease (PD), prion disease, and amyotrophic lateral sclerosis (ALS) (see, for example, Liu, W., et al., Role of Exosomes in Central Nervous System Diseases. Front Mol Neurosci, 2019. 12: p. 240 or Quek, C. and A. F. Hill, The role of extracellular vesicles in neurodegenerative diseases. Biochem Biophys Res Commun, 2017. 483(4): p. 1178-1186), mental disorders (Saeedi, S., et al., The emerging role of exosomes in mental disorders. Transl Psychiatry, 2019. 9(1): p. 122), cardiovascular diseases (Wang, Y., et al., Exosomes: An emerging factor in atherosclerosis. Biomed Pharmacother, 2019. 115: p. 108951), metabolic diseases (see, for example, Dini, L., et al., Microvesicles and exosomes in metabolic diseases and inflammation. Cytokine Growth Factor Rev, 2020. 51: p. 27-39 or Soazig, L. L., A. Ramaroson, and M. M. Carmen, Exosomes in metabolic syndrome, in Exosomes: A Clinical Compendium, L. R. Edelstein, et al., Editors. 2020, Academic Press. p. 343-356) and many more.

Exosome cargoes have been shown to consist of various biomolecules including proteins, lipids and nucleic acids. RNA species such as tRNA, mRNA, lncRNA, circular RNA and miRNA can potentially regulate gene expression in target cells and tissue. Exosomes produced by certain cell types have been shown to possess therapeutic properties. In this respect, mesenchymal stem cells (MSCs) isolated from different sources such as bone marrow, adipose tissue, and umbilical cord have emerged as particularly favourable. MSC-derived exosomes to have shown potential therapeutic effects in animal models of cornea, cardiovascular, Alzheimer's, Parkinson's and inflammatory bowel diseases, among others. In addition to endogenous cells, in vitro cultured pluripotent stem cells (PSCs) such as embryonic stem cells (ESCs) and induced pluripotent stem cells (iPS) have been shown to produce exosomes (Song, Y. H., et al., Exosomes Derived from Embryonic Stem Cells as Potential Treatment for Cardiovascular Diseases. Adv Exp Med Biol, 2017. 998: p. 187-206. or Jeske, R., et al., Human Pluripotent Stem Cell-Derived Extracellular Vesicles: Characteristics and Applications. Tissue Eng Part B Rev, 2020. 26(2): p. 129-144. Administration of cell-free iPS-derived exosomes is considered to be safer than iPS-derived cells due to the risk of tumour formation from residual undifferentiated cells (Riazifar, M., et al., Stem Cell Extracellular Vesicles: Extended Messages of Regeneration. Annu Rev Pharmacol Toxicol, 2017. 57: p. 125-154). Notably, therapeutic properties have also been demonstrated for exosomes isolated from differentiated derivatives of iPS. For example, treatment with exosomes purified from iPS-derived cardiomyocytes enhanced cardiac recovery in mouse model of myocardial infarction, with significant reduction in apoptosis and fibrosis compared to untreated animals. The exosomes also rescued in vitro cultures of iPS-cardiomyocytes from hypoxia and exosome biogenesis inhibition (Liu, B., et al., Cardiac recovery via extended cell-free delivery of extracellular vesicles secreted by cardiomyocytes derived from induced pluripotent stem cells. Nat Biomed Eng, 2018. 2(5): p. 293-303). In another study, exosomes from iPS-derived MSCs exosomes isolated from iPS-derived MSCs accelerated the proliferation of human dermal fibroblasts and human keratinocytes, and enhanced wound healing in in vitro scratch assays. There was no significant difference in the effects of these exosomes compared to those isolated from primary MSCs (Kim, S., et al., Exosomes Secreted from Induced Pluripotent Stem Cell-Derived Mesenchymal Stem Cells Accelerate Skin Cell Proliferation. Int J Mol Sci, 2018. 19(10).

Thus, in accordance with these reports, extracellular membranous vesicles or exosomes produced by CLiPS (either derived from CLMC or CLEC) or the differentiated derivatives of CLiPS of the present invention are considered useful for the treatment of diseases including the above-mentioned exemplary disease such as cancer, osteoarthritis, diseases of the central nervous system such as such as stroke, Alzheimer's disease (AD), Parkinson's disease (PD), prion disease, and amyotrophic lateral sclerosis (ALS), mental disorders or metabolic diseases.

In addition, taking advantage of their efficient cargo delivery ability, exosomes are actively pursued as delivery carriers for facilitating cellular uptake of various therapeutic agents such as microRNA, drugs, and peptides (see Antimisiaris, S. G., S. Mourtas, and A. Marazioti, Exosomes and Exosome-Inspired Vesicles for Targeted Drug Delivery. Pharmaceutics, 2018. 10(4), Liao, W., et al., Exosomes. The next generation of endogenous nanomaterials for advanced drug delivery and therapy. Acta Biomater, 2019. 86: p. 1-14 or Wang, X., et al., Cell-derived Exosomes as Promising Carriers for Drug Delivery and Targeted Therapy. Curr Cancer Drug Targets, 2018. 18(4): p. 347-354. In line with this, extracellular membranous vesicles or exosomes produced by CLiPS (either derived from CLMC or CLEC) or the differentiated derivatives of CLiPS of the present invention are considered can also be uses as delivery carriers for facilitating cellular uptake of therapeutic agents. Accordingly, the invention also encompasses the use of CLiPS or the differentiated derivatives of CLiPS for the purpose of delivery of exogenously loaded or transgenically expressed molecules.

Extracellular membranous vesicles and exosomes produced by CLiPS (either derived from CLMC or CLEC) or the differentiated derivatives of CLiPS can be isolated using respective methods described in the literature. Typically, exosomes are purified from the extracellular milieu into which they are secreted. Known methods for the isolation of exosomes include ultracentrifugation, ultrafiltration, size-exclusion chromatography, field-flow fractionation, polymer coprecipitation, immunoaffinity, microfluidics, or acoustic nanofilter. All these methods can be used for the isolation of exosomes produced by CLiPS or the differentiated derivatives of CLiPS described here.

The invention will be further illustrated by the following non-limiting Experimental Examples.

### EXPERIMENTAL EXAMPLES

### Example 1: Developing Suitable Electroporation Parameters for CLiPS

Electroporation according to the protocol described in Okita et al., supra, was found to be not working at all. No IPS colony was detected, when a reaction mixture of CLMC was electoporated with the episomal vectors pCXLE-hOCT3/4-shp53-F, pCXLE-hSK, and pCXLE-hUL, following the protocol of Okita et al, supra. For CLEC an average reprogramming efficiency (expressed in terms IPS colony counts) of only 0.2% after electroporation of CLMC with episomal vectors pCXLE-hOCT3/4-shp53-F, pCXLE-hSK, and pCXLE-hUL (Addgene plasmids #27077 (SEQ ID NO: 12), #27078 (SEQ ID NO: 13), #27080 (SEQ ID NO: 14) was found, following the protocol of Okita et al, supra. Accordingly, it was necessary to either develop from scratch a suitable electroporation protocol for CLMC derived CLiPS method or, in the case of CLEC, to provide a significantly improved electroporation protocol. For this purpose, the electric parameters such as number of electric pulses, duration time and voltage, constituting electroporation, were varied to develop useable electroporation conditions for CLSC. In this experiment, numerous different electroporation settings were tested on individual CLMC and CLEC samples, respectively, cultivated under the cell specific conditions as described here. After each electroporation, about 200.000 cells were plated in triplicates in a 6-well plate for cultivation. About 21 days after electroporation, CLSC colonies that had developed by then were counted to determine the survival rate. The survival rate was used to draw conclusions about electroporation efficiency. The percentage efficiency has been calculated as Colony number/200,000×100%.

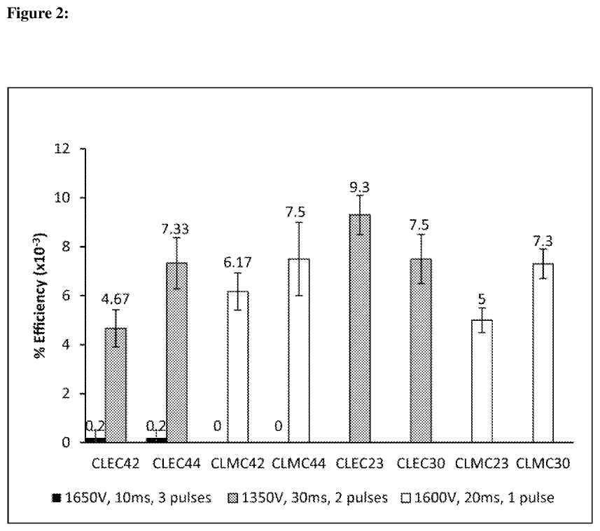

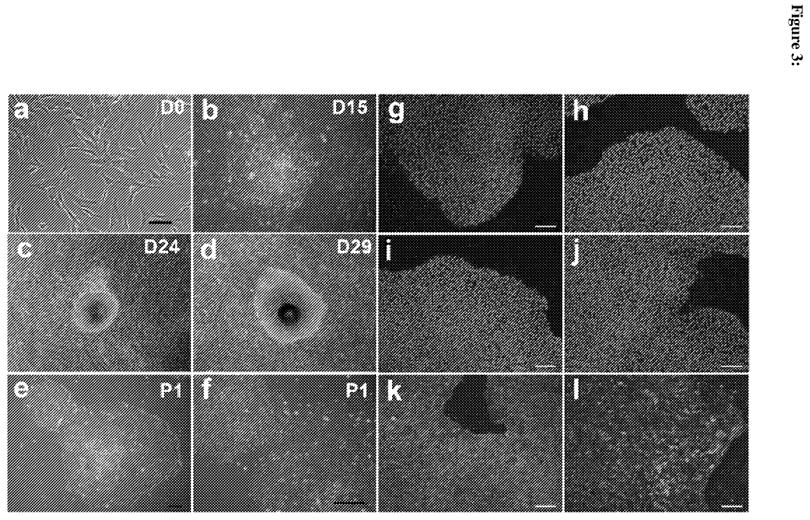

The results shown in Table 1 and FIG. 2 indicate that suitable electroporation conditions could be found for both CLMC and CLEC. The optimal electroporation setting for CLEC found here comprises 2 electric pulses each of 30 ms and 1350 V using an amount of 1.67 μg (plasmid) DNA of each of the three vectors (pCXLE-hOCT3/4-shp53-F, pCXLE-hSK, and pCXLE-hUL) for a number of 1×10⁶ cells. Four individual CLEC lines (CLEC42, CLEC44, CLEC23 and CLEC30) transfected with these settings exhibited a survival rate of 4.67%, 7.33%, 9.33% and 7.50%, respectively. In comparison to Okita et al., supra, the electroporation settings used for CLEC increased the electroporation efficiency about 23.35% for CLEC42 and about 36.65% for CLEC44. Thus, it has surprisingly been found that these electroporation parameters/settings increase the electroporation efficiency about 30% for CLEC on average compared to the conditions used by Okita et al for electroporation of human skin fibroblasts. Notably, the electroporation settings used here differ rather significantly from conditions reported for successful electroporation of epithelial cells such as corneal epithelial cells (1 electric pulse of 30 ms and 1300 V and a ratio of the amount of plasmid DNA (μg) to the number of cells (1×10⁶ cells) of 1:1 (cf. Png, E. et al. (2011), Journal of Cellular Physiology. United States, 226(3), pp. 693-699),

The effect of optimizing the electroporation protocol is even more significant regarding CLMC since, as stated above, electroporation according to Okita et al., supra, resulted in no surviving CLMC at all. It was found here that four individual CLMC lines (CLMC42, CLMC44, CLMC23 and CLMC30) were successfully transfected with 1 electric pulse of 20 ms and 1600 V and a ratio of the amount of plasmid DNA of each the three episomal vectors (pCXLE-hOCT3/4-shp53-F, pCXLE-hSK, and pCXLE-hUL) to the number of cells of 1.67 μg (plasmid) DNA to about 1×10⁶ CLMC. The resulting transgenic cells exhibited a survival rate of 6.17%, 7.50%, 5.00% and 7.33%, respectively. Notably, also the electroporation/transfection conditions found here to be the optimum for the generation of CLiPS from CLMC are different from electroporation conditions reported so far. Cf. in this context Sprangers, A. J., Freeman, B. and Ogle, B. M. (2011), pp. 62-66, for example, who examined possible negative effects of electroporation of human embryonic stem cell (hESC)-derived mesenchymal stem cells. So doing, Sprangers et al. found that transfecting a total of 4 μg (plasmid) DNA in 1×10⁶ mesenchymal stem cells using 1 electric pulse of 20 ms and 1400 V provided the optimum for MSC transfection. Thus, the present invention provides a unique and efficient protocol for CLEC and CLMC electroporation, respectively. The variations in the transfection efficiencies across the four individual CLSC lines (cells from different donors) are inter-individual variabilities being an inherent and documented feature of iPS derivation. To confirm the gender of the donor CLSC lines and CLiPS derived from them, a PCR amplification was performed with gene-specific primers on genomic DNA isolated from individual CLSC lines to determine the presence or absence of the DYS439 and SRY loci, which are both present on the Y chromosome. aSF4 adult skin fibroblasts, which is confirmed to be obtained from a male donor, was used as a positive control.

TABLE 1
Optimized electroporation conditions for generation of CLiPS

| | 1650 V, 10 ms, | | Optimized CLEC | | Optimized CLMC | |
|---|---|---|---|---|---|---|
| | 3 pulses | | protocol | | protocol | |
| | (Okita et al., 2011) | | 1350 V, 30 ms, 2 pulses | | 1600 V, 20 ms, 1 pulse | |
| | Ratio of the amount of DNA to number of cells [μg/1 × 10⁶ cells] for each vector | | | | | |
| | 1.67 μg/1 × 10⁶ cells | | 1.67 μg/1 × 10⁶ cells | | 1.67 μg/1 × 10⁶ cells | |
| | Av. | Av. % | Av. | Av. % | Av. | Av. % |
| | Colony | Efficiency | Colony | Efficiency | Colony | Efficiency |
| | count | (×10⁻³) | count | (×10⁻³) | count | (×10⁻³) |
| CLEC42 | 0.33 ± 0.58 | 0.2 ± 0.3 | 9.33 ± 1.53 | 4.67 ± 0.76 | — | — |
| CLEC44 | 0.33 ± 0.58 | 0.2 ± 0.3 | 14.67 ± 2.08 | 7.33 ± 1.04 | — | — |
| CLMC42 | 0 | 0 | — | — | 12.33 ± 1.53 | 6.17 ± 0.76 |
| CLMC44 | 0 | 0 | — | — | 15 ± 3 | 7.50 ± 1.50 |
| CLEC23 | — | — | 18.67 ± 1.53 | 9.33 ± 0.77 | — | — |
| CLEC30 | — | — | 15.00 ± 2.00 | 7.50 ± 1.00 | — | — |
| CLMC23 | — | — | — | — | 10.00 ± 1.00 | 5.00 ± 0.50 |
| CLMC30 | — | — | — | — | 14.67 ± 1.15 | 7.33 ± 0.58 |

### Example 2: Derivation of Transgene Integration- and Feeder-Free Human iPS

Cord lining epithelial cells (CLEC) and cord lining mesenchymal cells (CLMC) were isolated and supplied by CellResearch Corporation Pte Ltd, Singapore. CLEC and CLMC were thawed and propagated in their culture medium PTT-e3 and PTT-4, respectively. Adult skin fibroblasts from a healthy, 78 years old male Asian donor were purchased from CellResearch Corporation Pte Ltd and cultured in DMEM/10% FBS.

The culture medium PTT-4 consists of 90% (v/v) CMRL-1066 and 10% (v/v) FBS, while the medium PTTe-3 has the following composition:

| | |
|---|---|
| MCDB - 170/EpiLife | 200 ml/300 ml |
| DMEM | 250 ml |
| DMEM/F12 | 250 ml |
| Fetal Bovine Serum | 1% |
| Adenine | 0.05 to 0.1 mM |
| Hydrocortisone | 0.1 to 0.5 μM |
| Epidermal Growth Factor | 1 to 15 ng/ml |
| T3 (3,3′,5-Triiodo-L-thyronine sodium salt) | 0.1 to 5 ng/ml. |
| Cholera toxin vibrio cholerae | 1 × 10⁻¹¹M to 1 × 10⁻¹⁰M |
| Insulin | 1 to 7.5 μg/ml |
| TGF-alpha | 1.0 to about 10 ng/ml. |
| TGF-beta1 | 0.1 to 5 ng/ml |

Somatic reprogramming was performed using the conditions established in Example 1 and was further in a feeder-independent manner Log-phase cultures were harvested by dissociation with TrypLE Express (ThermoFisher Scientific) and 0.72 million cells were pelleted in a 1.5 ml centrifuge tube. The cell pellet was resuspended in 120 μL of Buffer R (Neon™ Transfection System 100 μL Kit, Thermo Fisher Scientific MPK10096). A cocktail containing 1.2 μg each of episomal vectors pCXLE-hOCT3/4-shp53-F, pCXLE-hSK, and pCXLE-hUL (Addgene plasmids #27077 (SEQ ID NO: 12), #27078 (SEQ ID NO: 13), #27080 (SEQ ID NO: 14), respectively) was added to the cells and mixed thoroughly (each vector was used in an amount of 1.67 μg (plasmid) DNA for a number of 1×10⁶ cells). The cell suspension was loaded into a 100 tit Neon® Tip and Neon electroporation was performed with the following parameters: adult skin fibroblasts—1,650 V, 10 ms, 3 pulses; CLEC—1350V, 30 ms, 2 pulses; CLMC—1600V, 20 ms, 1 pulse. Cells were immediately transferred into 6 ml of CLEC or CLMC medium containing 1 μM hydrocortisone (StemCell Technologies) and distributed equally into 3 wells of a Matrigel-coated 6-well plate. Two days later, the medium was switched to a 1:1 mixture of CLEC or CLMC medium and mTeSR1 supplemented with 1 μM hydrocortisone. On day 4 post-transfection, medium change was performed with the same medium. On day 6 post-transfection, medium was switched to complete mTeSR1 and hydrocortisone omitted from here on. Subsequently, medium change was performed every two days with mTeSR1. When iPS colonies reached about 1-2 mM in diameter (around Day 20 onwards), they were manually picked under bright field microscopy and each colony placed in a single well of Matrigel coated 24-well plate (Nunc). When cells in each well reached ˜50% confluence, they were detached with Dispase (StemCell Technologies) and transferred into a well of a Matrigel-coated 6-well plate. Subsequently, cells were passaged 1:3 by dissociation with 0.5 mM EDTA when they reached near confluence. Newly passaged cells were cultured overnight in medium containing 10 μM ROCK inhibitor Y-27632. In addition to mTeSR1, other commercial ES/iPS culture medium such as StemMACS™ iPS-Brew XF (Miltenyi Biotec) and TeSR-E8 (StemCell Technologies) have been used to maintain the iPS cultures.

Protocol for Generating CLiPS:
1. Actively dividing CLEC or CLMC cultured in T-75 flasks in their maintenance medium PTTe-3 and PTT-4, respectively, are harvested by dissociation using TrypLE Express (ThermoFisher Scientific).
2. Cells are counted and 0.72 million cells are aliquoted into a microfuge tube and pelleted.
3. The cell pellet is resuspended in 120 μL of Buffer R (Neon™ Transfection System 100 μL Kit, Thermo Fisher Scientific MPK10096). A cocktail containing 1.2 μg each of pCXLE-hUL, pCXLE-hSK, and pCXLE-hOCT3/4-shp53-F is added and mixed thoroughly.
4. The cell suspension is loaded into a 100 μL Neon® Tip. Electroporation is performed with the following parameters for CLEC: 1350V, 30 ms, 2 pulses and the following parameters for CLMC: 1600V, 20 ms, 1 pulse.
5. Cells are immediately transferred into 4 ml of CLEC or CLMC medium (PTTe-3 and PTT-4, respectively) containing 1 μM hydrocortisone and then distributed into 3 wells of a Matrigel coated 6-well plate.
6. Two days after electroporation, the medium is changed to a 1:1 (v/v) mixture of CLEC or CLMC medium (PTT-e3 and PTT-4, respectively) and mTeSR1 supplemented with 1 μM hydrocortisone.
7. Four days after electroporation, a medium replacement is performed with the same 1:1 (v/v) media mixture.
8. Six days after electroporation, medium is changed to mTeSR1 only. Hydrocortisone is omitted from here on.
9. Medium replaced is performed every two days
10. iPS colonies may start appearing as early as 2 weeks after transfection. When iPS colonies reach about 0.5 mm to about 1 mm in diameter (around Day 20 onward), they are manually picked under bright field microscopy and each colony is placed in a single well of Matrigel coated 24-well plate (Nunc).
11. After colony picking, medium change for isolated colonies is performed daily.
12. When cells in each well occupy about 50% of the culture surface, they are detached with Dispase (StemCell Technologies) and transferred into a well of a Matrigel coated 6-well plate.
13. Subsequently, cells are passaged 1:3 by dissociation using 0.5 mM EDTA when they reach about 70%-80% confluence. Newly passaged cells are cultured overnight in medium containing 10 μM ROCK inhibitor Y-27632.

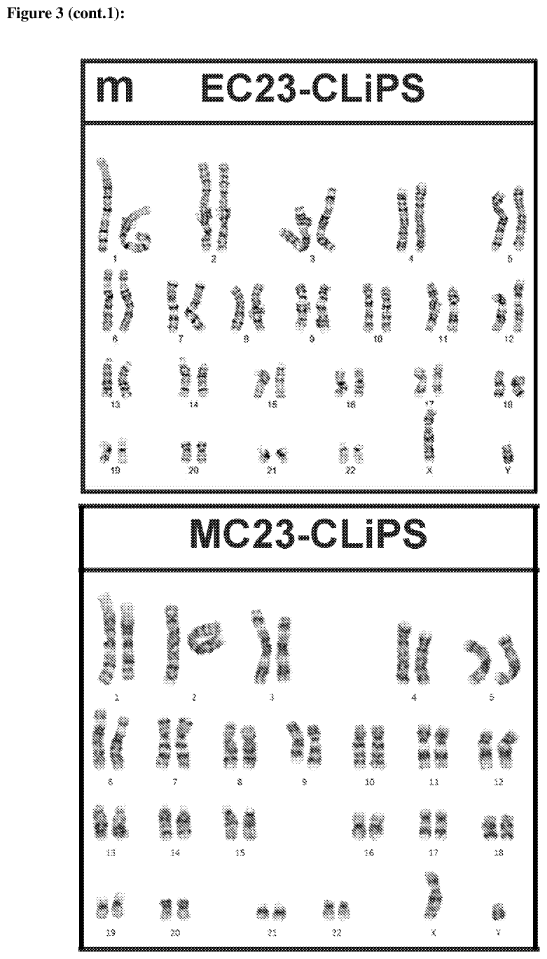

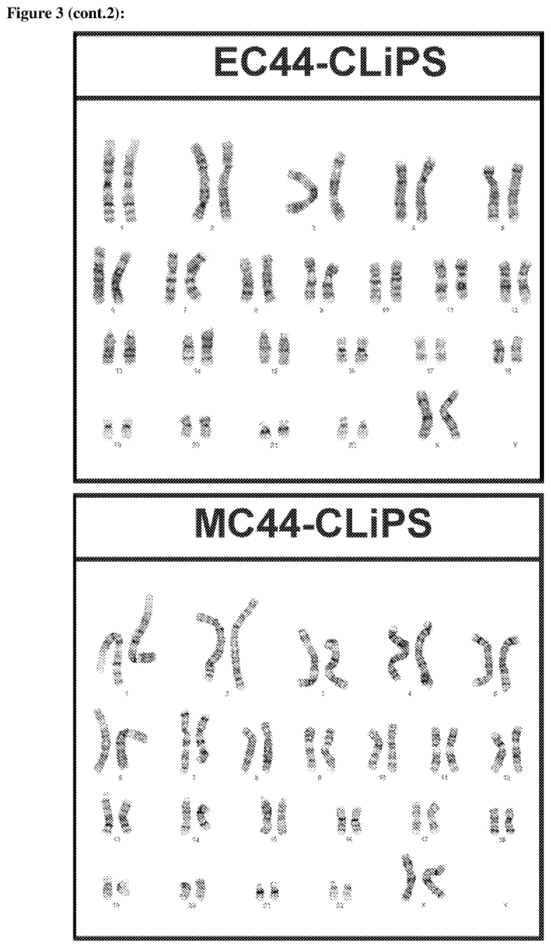

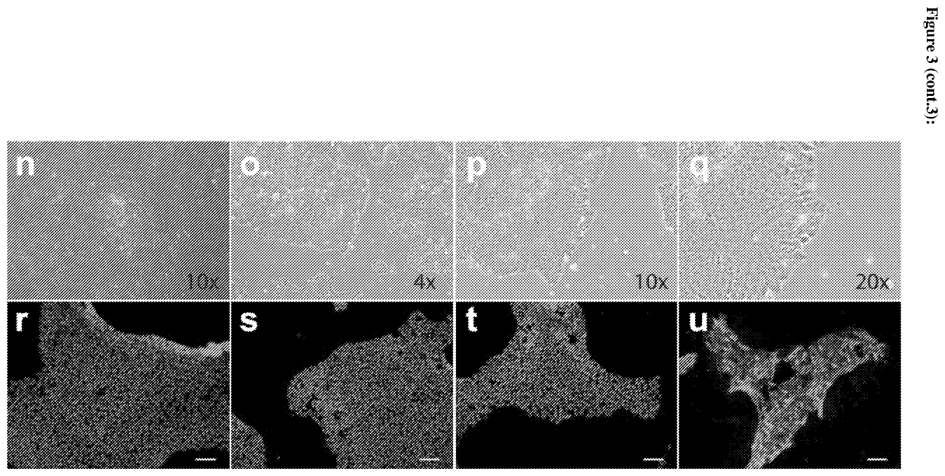

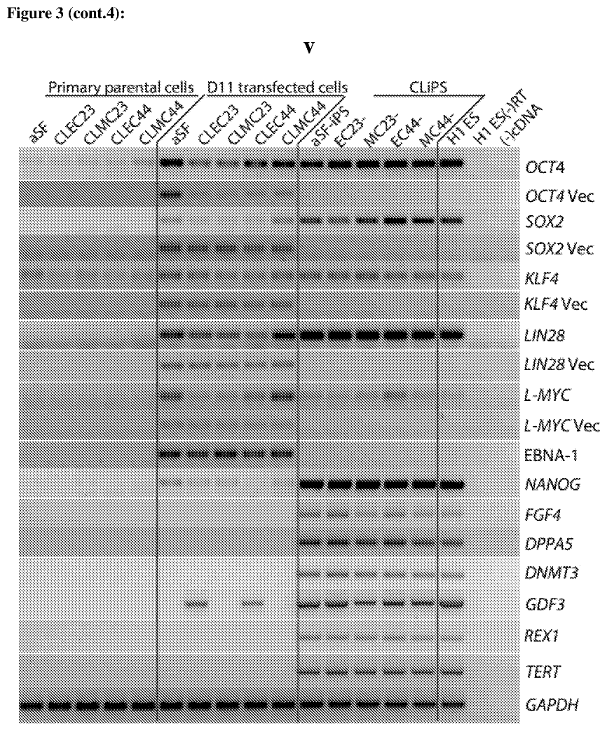

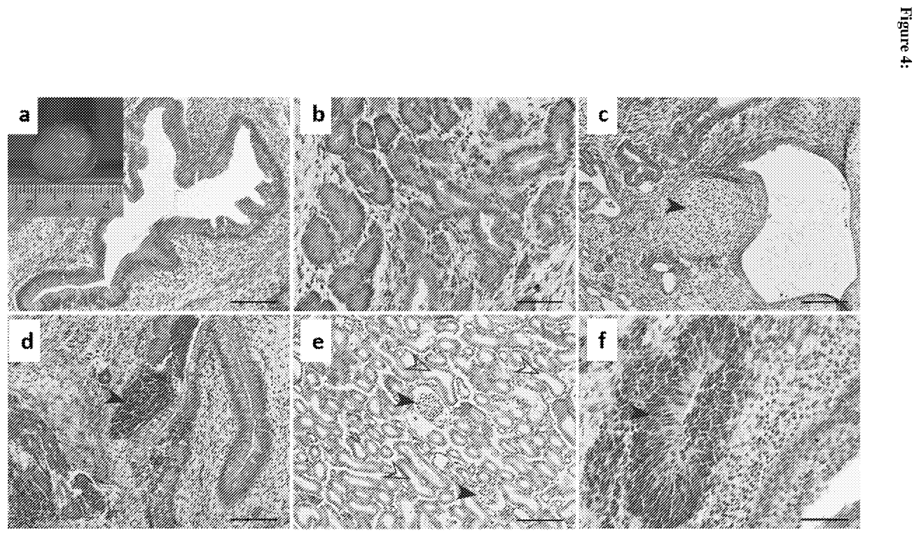

Following the protocol described above, small clusters of cells that look morphologically distinct from parental cells began to emerge starting at around Day 10. By Day 15, the cell clusters acquired defined edges (FIG. 3b) and discrete embryonic stem cell-like colonies appeared from Day 20 onwards (FIG. 3c and FIG. 3d). Colonies were picked when they reached 1-2 mm in diameter and expanded for characterization and storage. Expanded CLiPS exhibited cellular morphology indistinguishable from that of adult skin fibroblast-derived iPS or human embryonic stem cells (ES) with characteristic large nuclei and thin cytoplasm (FIG. 3e and FIG. 3f).

### Example 3: Derivation of cGMP-Compatible CLiPS (CLMSC-DTHN

To provide proof-of-concept that CLiPS can be produced under conditions compatible with human therapeutic applications, iPS were generated from a cGMP-grade CLMC line designated CLMSC-DTHN using the protocol described in WO2018/067071 for the production of the mesenchymal stem population of which 99% of the stem cell express the markers CD73, CD90 and CD105 while not expressing the markers CD34, CD45 and HLA-DR) cGMP quality reagents wherever possible. The reprogramming protocol is the same protocol described for CLMC in Example 2 but Matrigel, an extracellular matrix substrate prepared from Engelbreth-Holm-Swarm (EHS) mouse sarcoma cells, was replaced with recombinant human laminin-511 E8 fragment (iMatrix-511 SILK, ReproCELL), which is a defined, animal- and xeno-free substrate for coating cell culture vessels. In addition, mTeSR1 used for reprogramming and subsequent maintenance of CLiPS clones was replaced with cGMP mTeSR™1 (StemCell Technologies).

Under the conditions as described herein, CLMSC-DTHN were reprogrammed with comparable dynamics and efficiencies as CLMC (data not shown). At 10 days post-transfection with reprogramming vectors, small clusters of cells with compact morphology can be observed (FIG. 3n). These clusters developed into isolatable colonies from Day 20 onwards. Expanded colonies displayed the characteristic cellular morphology of human pluripotent stem cells (FIG. 3n-q).

Propagation and Cryopreservation of CLiPS

Sub-culturing of CLiPS (again using a media medium such as mTeSR1 or TeSR-E8 that is suitable for maintenance of iPS cells) is performed when cultures reached ˜90% confluence. Spent culture medium is aspirated off along with any overtly differentiated areas that may be present. Caution should be taken not to allow cells to be exposed to air for too long. The culture is rinsed once with prewarmed (37° C.) Dulbecco's Phosphate Buffered Saline (DPBS). Appropriate volume of rewarmed (37° C.) 0.5 mM EDTA solution is added to the culture according to the culture vessel—0.5 ml/well of a 24-well dish, 1 ml/well of a 6-well dish or 2 ml for a 6 cm dish. The culture is placed in an incubator at 37° C. for 5 min following which it is observed under a microscope. Cells should appear rounded but not detached from the surface. The duration of incubation at 37° C. varies with different CLiPS lines and may range from about 5-10 min. Incubation duration will be largely based on prior experience with each line. Following incubation, the EDTA solution is gentle aspirated off taking care not to dislodge the cells. Using a 1 ml pipettor, a medium such as mTeSR1 or TeSR-E8 containing ROCK inhibitor Y-27632 is dispensed directly onto the cells to dislodge them. The volume of medium used is dependent on the vessel size used—0.5 ml/well of a 24-well dish, 1 ml/well of a 6-well dish or 2 ml for a 6 cm dish. Gentle pipetting is repeated until most of the cells have been dislodged. The cell suspension is then transferred to a 15 ml Falcon tube. The culture vessel is rinsed with fresh medium and the rinse combined with the cell suspension in the Falcon tube. The cells in the tube are diluted to the appropriate volume for plating on new Matrigel-coated vessels. Split ratio may range from 1:3 to 1:10, depending on the density of the initial culture and also the growth rate of individual CLiPS lines.

For cryopreservation, cells are suspended in mTeSR1 or TeSR-E8 (or any other suitable culture medium) supplemented with 10% v/v of tissue culture-grade dimethyl sulfoxide (DMSO; e.g. Hybri-Max™, Sigma-Aldrich). The cell suspension is then aliquoted into appropriate numbers of cryovial. The density of cells per aliquot is dependent on the desired rate at which cell confluence is achieved upon thawing and culturing of the aliquot. Cryovials are then transferred to a slow freezing apparatus such as Mr. Frosty™ Freezing Container (Thermo Scientific) or CoolCell® Cell Freezing Containers (BioCision LLC) and placed overnight at −80° C. The next day, the cryovials are transferred to liquid nitrogen storage. It is not advisable to leave CLiPS aliquots at −80° C. for more than 24 hr. Several commercial freezing medium such as mFreSR™ (StemCell Technologies) and CryoStor® CS10 (Biolife Solutions) are also available for cryopreservation and may be used according to manufacturers' instructions.

### Example 4: Analysis of CLiPS Functionality

CLiPS functionality was determined by subjecting colony developing CLiPS to an immunofluorescent staining after electroporation. Thereby, the expression of pluripotent embryonic stem cell markers (OCT4, SOX2, KLF4, NANOG, SSEA-4, TRA-1-81) was analyzed. For this purpose, cells were fixed with 4% formaldehyde in phosphate buffer saline (PBS) for 15 min and subsequently washed 3 times for 5 min with PBS. For staining of intracellular or nuclear markers (OCT4, SOX2, KLF4, NANOG), cells were permeabilized with 0.1% Triton X-100 in PBS for 10 min, and blocked with FDB (5% FCS/1% NGS/1% BSA) for 1 h. For staining of surface markers (SSEA-4, TRA-1-81), the permeabilization step was omitted. Cells were incubated overnight with primary antibodies that were appropriately diluted with FDB at 4° C. followed by incubation with the appropriate fluorochrome-conjugated secondary antibodies at room temperature for 2 h. Stained samples were mounted in ProLong Diamond Antifade Mountant with DAPI (ThermoFisher Scientific).

Further, number and structure of the chromosomes within individual CLiPS lines were evaluated by performing a karyotype analysis and G-banding analysis, wherein the G-banding analysis was performed by the Cytogenetics Laboratory, KK Women's and Children's Hospital Pte. Ltd., Singapore.

Additionally, an RT-PCR analysis was performed to analyze the expression of reprogramming and pluripotent genes in primary parental cells, parental cells 11 days after vector transfection (D11 transfected cells) and CLiPS. For this purpose, total RNA was isolated from cell pellets using the RNeasy Mini or Plus Mini kits (Qiagen). Two μg of total RNA was treated with DNase I and used for cDNA synthesis with the RevertAid H Minus First Strand cDNA Synthesis Kit (Fermentas, Thermo Fisher Scientific). PCR reactions were set up as follows: 0.5 μl cDNA, 5 μl 2× MyTaq HS Mix (Bioline), 0.41 forward primer (10 μM), 0.41 reverse primer (10 μM), 4.41 PCR water. Thermal cycling was performed in an MJ Mini Thermal Cycler (Bio-Rad) with the following conditions: 1×95° C. 1 min, 30× (95° C. 15 s, Tm 15 s, 72° C. 15 s), 72° C. 1 min. Primer sequences and annealing temperature used (Tm) are provided in Table 2 below.

A qualitative expression analysis was performed by an agarose gel analysis, wherein the samples were loaded on a 2% agarose gel incorporated with SYBR Safe DNA stain (Thermo Fisher Scientific) in 1×TAE buffer and electrophoresed at 80V for 30 min. The gel images were captured using a ChemiDoc Imaging System (Bio-Rad).

TABLE 2
Primer sequences

| | | | | Amplified |
|---|---|---|---|---|
| | SEQ ID | | | fragment |
| Oligo Name | No | Sequence (5′ → 3′) | Tₘ | (bp) |
| hNanogF | 15 | AAGGTCCCGGTCAAGAAACAG | 55 | 237 |
| hNanogR | 16 | CTTCTGCGTCACACCATTGC | 55 | |
| hKlf4F | 17 | CCCACATGAAGCGACTTCCC | 55 | 169 |
| hKlf4R | 18 | AGGTCCAGGAGATCGTTGAAC | 55 | |
| hSox2F | 19 | TGGACAGTTACGCGCACATG | 55 | 214 |
| hSox2R | 20 | GAGTAGGACATGCTGTAGGTG | | |
| hOct4F | 21 | TGCGGCCCTTGCTGCAGAAG | 60 | 201 |
| hOct4R | 22 | GCTGCTGGGCGATGTGGCTG | | |
| Oct4VecF | 23 | ATGCATTCAAACTGAGGTAAGG | 55 | 127 |
| pCXLE-R2 | 24 | TAGCGTAAAAGGAGCAACATAG | | |
| Lin28F/VecF | 25 | CCATATGGTAGCCTCATGTCC | 55 | 126 |
| hLin28R | 26 | TCAATTCTGTGCCTCCGGGAG | | |
| Klf4VecF | 27 | ACCACCTCGCCTTACACATGAAG | 55 | 156 |
| pCXLE-R2 | 28 | TAGCGTAAAAGGAGCAACATAG | | |
| L-mycVecF | 29 | GGCTGAGAAGAGGATGGCTAC | 55 | 124 |
| pCXLE-2AR | 30 | AGTTTGTTTGACAGGAGCGAC | | |
| Sox2VecF | 31 | TCACATGTCCCAGCACTACC | 55 | 112 |
| pCXLE-2AR | 32 | AGTTTGTTTGACAGGAGCGAC | | |
| hL-mycF | 33 | AACCCAAGACCCAGGCCTGC | 60 | 135 |
| hL-mycR | 34 | GGTCTGCTCGCACCGTGATG | | |
| EBNA-1F | 35 | GAAATGGCCTAGGAGAGAAG | 55 | 214 |
| EBNA-1R | 36 | CAGCCAATGCAACTTGGACG | | |
| hGDF3F | 37 | CTTATGCTACGTAAAGGAGCTGGG | 60 | 633 |
| hGDF3R | 38 | TTGTGCCAACCCAGGTCCCGGAAG | | |
| hREX1F | 39 | TATCAGATCCTAAACAGCTCGCAG | 55 | 308 |
| hREX1R | 40 | CGTACGCAAATTAAAGTCCAGAG | | |
| hFGF4F | 41 | ACTACAACGCCTACGAGTCCTAC | 60 | 372 |
| hFGF4R | 42 | GTTGCACCAGAAAAGTCAGAGTTG | | |
| hDPPA5F | 43 | ATATCCCGCCGTGGGTGAAAGTTC | 60 | 243 |
| hDPPA5R | 44 | ACTCAGCCATGGACTGGAGCATCC | | |
| hTERTF | 45 | CCTGCTCAAGCTGACTCGACACCGTG | 65 | 446 |
| hTERTR | 46 | GGAAAAGCTGGCCCTGGGGTGGAGC | | |
| hDNMT3BF | 47 | TGCTGCTCACAGGGCCCGATAC | 60 | 242 |
| hDNMT3BR | 48 | TCCTTTCGAGCTCAGTGCACCAC | | |
| hGAPDHF | 49 | CTGGCGCTGAGTACGTCGTGG | 60 | 200 |
| hGAPDHR | 50 | GCAGTTGGTGGTGCAGGAGGC | | |

The results show that CLiPS showed robust expression of the human embryonic stem cell (hES) markers KLF4, NANOG, OCT4, SOX2, SSEA4, and TRA-1-60 as demonstrated by antibody staining (FIG. 3g-l). G-banding analysis showed that CLiPS maintained a normal karyotype up to 17 passages from colony picking (FIG. 3m). RT-PCR analysis of gene expression in parental cells, Day 11 post-transfected cells and expanded iPS clones revealed that activation of endogenous OCT4, SOX2, KLF4, LIN28 and L-MYC genes has replaced the roles of vector-driven expression of these genes for the maintenance of pluripotency in fully reprogrammed CLiPS (FIG. 3v). The induction of endogenous NANOG loci, a crucial gene for somatic reprogramming, was evident at Day 11 post-transfection. The absence of detectable levels of EBNA-1 transcripts in CLiPS clones suggests that plasmid vectors have been lost from these cells. The expression of additional hES-specific genes GDF3, DPPA5, DNMT3, FGF4, and REX-1 in CLiPS further confirms their hES-like molecular phenotype. TERT, which encodes the catalytic reverse transcriptase subunit of telomerase that is essential for the regulation of self-renewal and the maintenance of pluripotency, is expressed in CLiPS at levels identical to that in H1 hES.

### Example 5: Expression Analysis of Pluripotent Embryonic Stem Cell Markers in CLiPS-DTHN

To analyze the expression of pluripotent embryonic stem cell markers (Oct4, Sox2, Klf4, Nanog) indicating pluripotency, developing CLMSC-DTHN were subjected to an immunofluorescent staining after electroporation. The immunofluorescent staining protocol was the same protocol described for CLiPS in Example 4.

The results show that CLMSC-DTHN express the pluripotent stem cell markers NANOG, OCT4, SOX2 and TRA-1-81 (FIG. 3r-u) at levels indistinguishable from their non-GMP counterparts. Thus, CLMSC-DTHN may provide the same embryonic properties non-GMP-derived CLiPS entail.

### Example 6: Determining CLiPS Pluripotency

The pluripotency of CLiPS and aSF-iPS was evaluated by teratoma formation assay in NOD-SCID mice. For this purpose, 1×10⁶ CLiPS cells were pelleted, resuspended in 0.1 ml of ice-cold Matrigel and injected into the dorsal flank of 6-8 week old NOD/MrkBomTac-Prkdc^{scid} mice. Mice were sacrificed after 3 month later and teratomas harvested for histological analysis, wherein Paraffin wax sectioning and hematoxylin and eosin staining were performed using standard techniques.

The results show that palpable tumors developed in some mice beginning from 1 month following subcutaneous injection of iPS into the dorsal flank of mice. Histological analysis of the teratomas isolated 3 months after injection revealed spontaneous differentiation of CLiPS into tissues of endodermal, mesodermal and ectodermal lineages (FIG. 4a-f).

### Example 7: Differentiation of CLiPS into Dopaminergic Neurons

As an important prerequisite for potential future therapeutic application of CLiPS, it is necessary to demonstrate their capability to differentiate into specific tissue type under defined in vitro conditions. For dopaminergic neuronal differentiation, the midbrain floor plate induction protocol described in Kriks, S., et al, Nature, 2011. 480(7378): p. 547-51 was used for the differentiation of iPS into dopaminergic neuroprogenitors and neurons. Briefly, iPS were plated at a density of 3.5-4.0×10⁴ cells per cm² on Matrigel (Corning) coated dishes and cultured for 5 days in knockout serum replacement medium (KSR) containing Knock-Out DMEM, 15% knockout serum replacement, 1× GlutaMAX and 10 mM β-mercaptoethanol. From Day 5, KSR medium was transitioned stepwise to N2 medium as described in Tomishima “Midbrain dopamine neurons from hESCs.” 2012 Jun. 10. In: StemBook. Cambridge (Mass.): Harvard Stem Cell Institute; 2008. Available from: https://www.ncbi.nlm.nih.gov/books/NBK133274/doi: 10.3824/stembook.1.70.1. On day 11, media was changed to NB27 medium composed of Neurobasal medium, 2% B27 minus vitamin A and 1× GlutaMAX and supplemented with CHIR (until day 13), BDNF (brain-derived neurotrophic factor, 20 ng/ml; Miltenyi), ascorbic acid (0.2 mM, Sigma), GDNF (glial cell line-derived neurotrophic factor, 20 ng/ml; Miltenyi), TGFβ3 (transforming growth factor type β3, 1 ng/ml; R&D), dibutyryl cAMP (0.5 mM; Santa Cruz Biotechnology), and DAPT (10 nM; Tocris) for 9 days. On day 20, cells were dissociated using Accutase (Gibco) and replated at high cell density (3-4×10⁵ cells per cm²) on dishes pre-coated with poly-L-ornithine (PLO; 15 mg/ml)/laminin (1 μg/ml)/fibronectin (2 μg/ml) in NB27 medium supplemented with 10 μM ROCK inhibitor Y-27632. Cultures were maintained in NB27 medium with medium replacement every other day until the desired endpoint. Differentiated cells were analysed for expression of cell specific markers at this stage. For this purpose, cryosectioning was performed, wherein slides containing the sections were dehydrated by incubation at 37° C. for 30 min, cooled to room temperature and washed 3 times with TBST. Section permeabilization, blocking, antibody staining and mounting were performed as described in Example 4. Primary antibodies from the same host species were used, a fluorochrome-conjugated monovalent antibody (Jackson ImmunoResearch) was used to saturate the first primary antibody before sequential incubation with the second primary antibody and conjugated secondary antibody.

The results show that dopaminergic neurons were obtained from CLiPS and asF5-iPS using this protocol. The antibody staining revealed that almost 90% of the cells coexpressed the floor-plate marker FOXA2 and the roof plate marker LMX1A (FIG. 4k, k′, k″), a definitive hallmark of midbrain DA neuron precursors. Further differentiation yielded abundant mature neurons as indicated by TUJ1 staining, out of which approximately 30-50% coexpressed the dopaminergic marker Tyrosine Hydroxylase (TH) (FIG. 4l, l′, l″). Electrophysiological analysis of CLiPS-derived neurons at Day 45 of differentiation demonstrated that the cells exhibited mature functional properties, with trains of action potential displaying the voltage sag response characteristic of mature mesencephalic DA neurons upon injection of hyperpolarizing currents (FIG. 4m).

### Example 8: Differentiation of CLiPS into Hepatocytes

As an important prerequisite for potential future therapeutic application of CLiPS, it is necessary to demonstrate their capability to differentiate into a desired target cell type, or a specific tissue type under defined in vitro conditions. For hepatic differentiation, a protocol that was originally developed for the differentiation of human embryonic cells (ES) on mouse feeder layer (Medine, C. N., et al., J Vis Exp, 2011(56): p. e2969) was adapted for CLiPS and asF-iPS differentiation in mTeSR1 on Matrigel. A modification is that when iPS cultures reached 20-30% confluency, DMSO was supplemented to 2% and incubated for 24 h. When cultures reached confluency of ˜30%-60%, definitive endoderm formation was induced by replacing the mTeSR1 with priming medium (RPMI 1640-B27 supplemented with 100 ng/mL Activin A and 50 ng/mL Wnt3a). Cultures were maintained in priming medium for 3 days with medium change every 24 h. After 72 h in priming medium, the medium was switched to SR-DMSO (80% KO-DMEM, 20% KO-SR, 0.5% L-glutamine, 1% non-essential amino acids, 0.1 mM β-Mercaptoethanol and 1% DMSO) for 5 days with medium change every 48 hours). At day 8, cultures were switched to L-15 maturation and maintenance medium (Leibovitz L-15 medium, 8.3% tryptose phosphate broth, 8.3% heat inactivated FBS, 10 μM hydrocortisone 21-hemisuccinate, 1 μM Insulin (bovine pancreas), 1% L-Glutamine, 0.2% ascorbic acid) supplemented with 10 ng/mL hHGF and 20 ng/mL OSM for 9 days (changing medium every 48 hours). Differentiated cells were again analysed for expression of cell specific markers at this stage. For this purpose, cryosectioning was performed as described in Example 7.

The results show that hepatocyte-like cells were obtained from CLiPS and asF5-iPS using this protocol. The antibody staining revealed the expression of the hepatocyte markers alpha-fetoprotein (AFP; FIG. 4g, g′, g″), Cytokeratin 18 (CK18) and Human Serum Albumin (HSA; FIG. 4h, h′, h″) after 17 days of differentiation. A majority of the differentiated cells exhibited a polygonal shape characteristic of hepatocytes. In addition, staining with Oil Red O showed abundant lipid droplet accumulation in the cells, a hallmark of cultured hepatocytes (FIG. 4i, i′, i″).

### Example 9: Differentiation of CLiPS into Cardiomyocytes

As an important prerequisite for potential future therapeutic application of CLiPS, it is necessary to demonstrate their capability to differentiate into specific tissue type under defined in vitro conditions. For cardiomyocyte differentiation, the protocol for cardiomyocyte differentiation of iPS was adapted from the protocol described in Lian, X., et at, Proc Natl Acad Sci USA, 2012. 109(27), p. E1848-57. iPS maintained on Matrigel in mTeSR1 were dissociated into single cells with StemPro Accutase (Thermo Fisher Scientific) at 37° C. for 5 min and then seed onto a Matrigel-coated cell-culture dish at 1×10⁵-2×10⁵ cell/cm² (5×10⁵ cells per 24-well) in mTeSR1 supplemented with 5 μM ROCK inhibitor (Y-27632; Stemgent) for 24 h. In a modification, when cells reached ˜80% confluency, medium was switched to mTeSR1 supplemented with 2% DMSO. When cells reached confluence, they were treated with CHIR99021 in RPMI/B27-insulin for 24 h. In another modification, the concentration of CHIR99021 was lowered to 5 μM from the original 12 μM at this stage. The next day, the medium was changed to RPMI/2% B27 without insulin. Two days later, half of the old medium was combined with an equal volume of fresh medium containing 10 μM IWP2 (Tocris). The remaining medium in the wells was discarded and the mixture added to the cultures. Two days later, medium was switched to RPMI/2% B27 without insulin only. After 48 hr, cultures were maintained in RPMI/2% B27 with medium change every 3 days until the desired endpoint. Beating cardiomyocytes were fixed and stained for cell specific markers as described in Example 7.

The results show that cardiomyocytes were obtained from CLiPS and asF5-iPS using this protocol. The antibody staining revealed the expression of the spontaneously contracting cardiomyocytes were observed beginning from 8 days of differentiation Immunofluorescent antibody staining for the functional cardiac markers Myosin regulatory light chain 2a (MLC2a), cardiac troponin I (cTnI) and alpha-actinin (αACT) revealed sarcomeric structures within differentiated cardiomyocytes (FIG. 4j, j′, j″). No noticeable difference in differentiation efficiency was observed between.

### Example 10: Differentiation of CLiPS into Oligodendrocytes

To further demonstrate the ability of the induced pluripotent stem cells of the invention to differentiate into a given target cell type, CLiPS were differentiated into oligodendrocytes. Oligodendrocyte differentiation of CLiPS and asF-iPS was performed according to the protocol of Douvaras, P. and V. Fossati, Nat Protoc, 2015. 10(8): p. 1143-54. Further, cryosectioning was performed as described in Example 7 to analyse the expression of cell specific markers.

At Day 75 of differentiation, clusters of Olig2-positive oligodendrocyte precursor cells (OPCs; FIG. 4n) and also 04-positive late OPCs were obtained (FIG. 4o).

### Example 11: Immunogenicity Analysis

To gain some insight into the immunogenicity of CLiPS and their neural derivatives, the expression of a panel of immunogenicity related markers by these cells was assessed by flow cytometric analysis. For this purpose, primary cells and Day 25 differentiated DA NPCs were harvested by dissociation with TrypLE Express while iPS cultures were harvested by dissociation with 0.5 mM EDTA. Cells were resuspended in 1× Ca²⁺- and Mg²⁺-free DPBS containing 0.1% bovine serum albumin (BSA) to 5 million cells/ml. 100 μl of cells was stained with the appropriate conjugated antibodies or their isotype controls in the dark for 30 min on ice. For HLA-E and HLA-G staining, cells were permeabilized with BD Phosflow Perm/Wash Buffer I (BD Biosciences) according to manufacturer's instructions prior to staining. Following staining, cells were washed 2× in 1× Ca²⁺- and Mg²⁺-free DPBS/5 mM EDTA, fixed with 1% paraformaldehyde for 1 hr in the dark and then were washed 2× in 1× Ca²⁺- and Mg²⁺-free DPBS/5 mM EDTA. Cells were resuspended in 0.5 ml 1× Ca²⁺- and Mg²⁺-free DPBS/5 mM EDTA and analysed on a flow cytometer. Stained primary cells and iPS were analysed on a FACSCalibur while stained dopaminergic neuronal progenitor cells (NPCs) were analysed on a FACSCanto II instrument (both from BD Biosciences). Data was analysed using FlowJo software package (FlowJo LLC). Antibodies used are listed in Table 3.

TABLE 3
Antibodies used for flow cytometry

| | | | | BioLegend |
|---|---|---|---|---|
| Antigen | Isotype | Conjugate | Dilution | Cat. No. |
| CD40 | Mouse IgG1, κ | FITC | 1:20 | 303604 |
| CD80 | Mouse IgG1, κ | FITC | 1:20 | 305206 |
| CD86 | Mouse IgG2b, κ | Alexa Fluor 488 | 1:20 | 305414 |
| HLA-A, B, C | Mouse IgG2a, κ | PE | 1:20 | 311406 |
| HLA-DR | Mouse IgG2a, κ | PE | 1:20 | 307606 |
| HLA-E | Mouse IgG1, κ | PE | 1:20 | 342604 |
| HLA-G | Mouse IgG2a, κ | APC | 1:20 | 335910 |
| NCAM/CD56 | Mouse IgG1, κ | APC/Cy7 | 1:20 | 318332 |
| Isotype control | Mouse IgG1, κ | FITC | 1:20 | 400108 |
| Isotype control | Mouse IgG2b, κ | Alexa Fluor 488 | 1:20 | 400329 |
| Isotype control | Mouse IgG1, κ | PE | 1:20 | 400112 |
| Isotype control | Mouse IgG2a, κ | PE | 1:20 | 404212 |
| Isotype control | Mouse IgG1, κ | APC | 1:20 | 400120 |
| Isotype control | Mouse IgG2a, κ | APC | 1:20 | 400222 |
| Isotype control | Mouse IgG1, κ | APC/Cy7 | 1:20 | 400128 |

MHC Class I HLA-A, -B and -C and MHC Class II HLA-DR molecules are known to be important for alloimmune response. The results show that HLA-ABC was expressed across all iPS samples but a markedly reduced level was observed for EC23-CLiPS (FIG. 6a). HLA-DR expression was absent in all iPS samples (FIG. 6b), consistent with previous reports of negligible HLA-II expression in iPS (Säljö, K., et al., Sci Rep, 2017. 7(1): p. 13072 and Chen, H. F., et al., Cell Transplant, 2015. 24(5): p. 845-64). T cell co-stimulatory molecules CD40, CD80, and CD86 play an important role in activating T cells during alloimmune response. Of the three molecules examined, only CD40 was expressed on iPS, with the lowest level expressed by asF-iPS and the highest level expressed by MC23-CLiPS compared to the rest (FIG. 6a). As the tolerogenic HLA-E and HLA-G have been reported to be expressed on CLMC (Deuse, T., et al., Cell Transplant, 2011. 20(5): p. 655-67) and CLEC (Zhou, Y., et al., Cell Transplant, 2011. 20(11-12): p. 1827-41), the expression of these antigens by CLiPS was also investigated. Analysis of permeabilized cells revealed only marginal expression of HLA-E in MC23-CLiPS and EC44-CLiPS, and below detectable levels in other samples. Next, the expression profiling of the entire panel of markers on Day 25 DA differentiation cultures was repeated. Analysis was performed on neural cells populations gated on positive staining for NCAM. NCAM+ fractions exceeded 97% for all samples, with asF-iPS and EC23-CLiPS showing comparable differentiation efficiencies of 99.5% (FIG. 6b). HLA-ABC was expressed by all NPC samples but at generally lower levels compared to their parental iPS (FIG. 6c). EC23-CLiPS-derived NPCs expressed the lowest level of HLA-ABC amongst the samples, mirroring the trend displayed by its parental iPS. The level of HLA-ABC expression on MC23-CLiPS was reduced upon its differentiation to NPCs. CD40 expression was downregulated across all NPC samples, with only EC23-iPS- and EC44-iPS-derived NPCs displaying slight expression. HLA-E expression was absent in all NPC samples but slight upregulation of HLA-G was observed in asF-iPS- and EC23-iPS-derived NPCs. These results indicate reduced immunogenity in CLiPS.

### Example 12: Transplantation of CLiPS-Derived Dopaminergic Neurons in Fully Immunocompetent Mice Models of Parkinson's Disease

Previous studies have shown that dopaminergic neurons generated from human embryonic stem cells and iPS using various protocols can engraft in rodent (Kriks, S., et al, Nature, 2011. 480(7378): p. 547-51; Hargus, G., et al., Proc Natl Acad Sci USA, 2010. 107(36): p. 15921-6; Doi, D., et al., Stem Cell Reports, 2014. 2(3): p. 337-50; Grealish, S., et al., Cell Stem Cell, 2014. 15(5): p. 653-65; Kirkeby, A., et al., Cell Rep, 2012. 1(6): p. 703-14; Qiu, L., et al., Stem Cells Transl Med, 2017. 6(9): p. 1803-1814; Rhee, Y. H., et al., J Clin Invest, 2011. 121(6): p. 2326-35; Samata, B., et al., Nat Commun, 2016. 7: p. 13097; Wakeman, D. R., et al., Stem Cell Reports, 2017. 9(1): p. 149-161) and non-human primate (Kriks, S., et al, Nature, 2011. 480(7378): p. 547-51; Hargus, G., et al., Proc Natl Acad Sci USA, 2010. 107(36): p. 15921-6; Wakeman, D. R., et al., Stem Cell Reports, 2017. 9(1): p. 149-161; Daadi, M. M., et al., PLoS One, 2012. 7(7): p. e41120; Kikuchi, T., et al., Nature, 2017. 548(7669): p. 592-596) models of Parkinson's Disease (PD). In all these studies, animals were either immunocompromised or otherwise immunosuppressed pharmacologically to prevent graft rejection. The need for immunocompromised or immunosuppressed animals was only obviated when transplantation was performed using either autologous (Morizane, A., et al., Stem Cell Reports, 2013. 1(4): p. 283-92; 4. Hallett, P. J., et al., Cell Stem Cell, 2015. 16(3): p. 269-74; Wang, S., et al., Cell Discov, 2015. 1: p. 15012; Emborg, M. E., et al Cell Rep, 2013. 3(3): p. 646-50; Sundberg, M., et al., Stem Cells, 2013. 31(8): p. 1548-62) or MHC-matched allogenic (Morizane, A., et al., 2017. 8(1): p. 385) iPS-derived cells.

To demonstrate the engraftability of CLiPS-derived DA NPCs differentiated using the method of the present invention, transplanted Day 25 NPCs differentiated from asF-iPS, EC23-CLiPS and MC23-CLiPS were transplanted into immunocompromised NOD-SCID mice (n=3). In this context, it is noted that all animal experiments were conducted according to protocols approved by the Institutional Animal Care and Use Committee (IACUC) of the National Neuroscience Institute (NNI), Singapore.

To test immunogenicity of CLiPS-derived DA NPCs, a transplantation needs to be performed on PD models. For this purpose, 6-hydroxydopamine (6-OHDA) unilateral lesion mouse models were generated. The unilateral 6-OHDA lesion is an established method for rodents and comprises the injection of 6-OHDA in the rodent brain causing motoric dysfunctions characterized by rotational asymmetry with the degree (Bagga, V., Dunnett, S. B. and Fricker, R. A. (2015) Behavioural Brain Research. Elsevier B. V., 288, pp. 107-117). In the present invention, 6-OHDA lesions were induced in NOD/MrkBomTac-Prkdc^{scid} mice (4 weeks old) purchased from InVivos Pte Ltd and maintained under SPF conditions at the Animal Research Facility, NNI and male C57BL/6NTac mice (6-8 weeks old) purchased from InVivos Pte Ltd., wherein the mice used for this experiment were fully immunocompetent and no immunosuppression was administered prior to or following transplantation.

To generate the mouse PD model, 7.5 μg of 6-OHDA (Sigma, Merck-Millipore; dissolved at 2.5 mg/ml in 0.9% NaCl containing 0.2% ascorbic acid) was delivered into the left striatum by stereotaxic injection at the following coordinates: anterior-posterior (AP)+0.5 mm; medial-lateral (ML) −1.8 mm from Bregma; dorsal-ventral (DV) −3.0 mm from cranium. After two weeks of acclimatization, transplantation of 3 NPCs samples (i.e. NPCs derived from asF-iPS, EC23-CLiPS and MC23-CLiPS) were transplanted into the striatum of immunocompetent, 6-OHDA lesioned C57BL/6 mice by stereotaxic injection was performed on mice models considered as successfully lesioned.

To determine suitable models for transplantation, apomorphine-induced rotations were scored and mice displaying >6 rotations per min were used for transplantation. For transplantation, Day 25 dopaminergic progenitor cells were harvested by dissociation and resuspended to ˜1.25×10⁵ cells/μ1 in HBSS supplemented with 10 ng/mL of BDNF, 10 ng/mL of GDNF. Two μL of cell suspension was injected into lesioned mice at the following coordinates: AP +0.5 mm; ML −2.0 mm, and DV −2.8 mm from skull. To assess whether transplanted NPCs can integrate and mediate functional benefits in lesioned animals, rotation asymmetry tests were performed at 2-weekly intervals. Rotation assays were performed every 2 weeks up to 9 months, wherein mice were injected intraperitoneally with 0.05 mg/kg of apomorphine dissolved in 0.9% NaCl containing 0.1% w/v ascorbic acid. Rotations were recorded using a digital camera and counted manually. Batches of animals were sacrificed at 1, 6 and 9 months after transplantation by terminal anesthesia.

Six months after transplantation, NPC-transplanted, sham-injected and non-manipulated mice were evaluated for striatal dopamine transporter (DAT) activity by positron-emission tomography (PET) imaging using the radioligand (2-[18F]Fluoroethyl 8-[(2E)-3-iodoprop-2-en-1-yl]-3-(4-methylphenyl)-8-azabicyclo[3.2.1] octane-2-carboxylate) ([18F]FE-PE2I). Animals were fasted for 3 hours prior to the imaging sessions Animals were kept warm during scanning with integrated hot-air channels from the imaging bed. The respiratory rate and temperature were monitored throughout the entire scan session to ensure adequate level of anaesthesia. Mice were imaged using the nanoScan PET/MRI scanner (Mediso Ltd., Hungary) at the SingHealth Experimental Medicine Centre (SEMC). This scanner is equipped with 12 detector modules with an axial field of view (FOV) of 94 mm and a transaxial FOV of 94 mm or 120 mm diameter in 1:3 and 1:5 coincidence modes, respectively Animals were placed head first in a prone position and a 3D dynamic PET scan was performed for 62 min with frames of increasing durations i.e. 4 at 10 sec, 4 at 20 sec, 3 at 1 min, 7 at 3 min and 6 at 6 min) following intravenous injection of 3.57-10.61 MBq of [18F]FE-PE2I in a maximum volume of 0.1 ml via the tail vein. [18F]PE-PE2I was synthesized at the Singapore Radiopharmaceuticals Pte Ltd. MRI images were used for the attenuation correction of the PET scans and as structural reference for the PET images in data analysis. Thus, T1-weighted MRI images were acquired using the MRI component of the nanoScan PET/MRI scanner. The integrated mouse head coil covers the entire brain during the MRI scan. Slices of 0.6 mm were obtained using a 3D GRE EXT sequence: 64-mm square FOV, 128×128 matrix, 20-ms repetition time (TR), 2.3-ms echo time (TE), 25 degree flip angle. All image and kinetic analyses for the [18F]FE-PE2I PET images were performed using PMOD (version 3.5; PMOD Technologies). All PET images were first automatically registered to the MRI images using the FUSION tool in PMOD. The MRI images were then manually registered to the T2-weighted mouse template (M. Mirrione, C57BL/6J mice; Ma, Y., et al., Neuroscience, 2005. 135(4): p. 1203-15; Mirrione, M. M., et al., Neuroimage, 2007. 38(1): p. 34-42), which contains a volume of interest (VOI) template with 20 regions. The accuracy of the manual registration was accessed and verified by two different persons. Finally, the combined transformation matrix was applied to transform the PET images to the MRI mouse template. VOIs for left and right striatum and cerebellum were used for the analysis. In order to reduce the errors from misregistration and misdefinition (He, B. and E. C. Frey, Phys Med Biol, 2010. 55(12): p. 3535-44), 3D erosion with one voxel was applied to the obtained VOIs. [18F]FE-PE2I binding was quantified using the non-invasive reference tissue models, since they are equally accurate as compared to the kinetic analyses with the arterial input function (Varrone, A., et al., Nucl Med Biol, 2012. 39(2): p. 295-303). The binding potential (BPnd) values were calculated using the simplified reference tissue model (SRTM) (Lammertsma, A. A. and S. P. Hume, 1996. 4(3 Pt 1): p. 153-8) with the cerebellum as the reference. The regional time activity curves (TACs) were also extracted from the VOIs of striatum and cerebellum. Anesthesia was induced with 5% isoflurane in 100% 02 and maintained with 1.5-2% isofluorane during the imaging.

Brain sections of mice were analyzed for the presence of microglia/macrophages, as these cells are known to play important roles in allograft and xenograft rejection in the CNS (Hoornaert, C. J., et al., Stem Cells Transl Med, 2017. 6(5): p. 1434-1441), For this purpose, an immunostaining for the microglia/macrophages-specific marker Iba1 was performed after transcardial perfusion with 4% PFA. For this purpose, PFA perfused brains were post-fixed overnight in 4% PFA followed by equilibration in 15% and 30% w/v sucrose solution in PBS until they settled to the bottom of the tubes. Brains were embedded in OCT freezing medium and 18 μm sections were cut on a CM3050 S cryostat (Leica Biosystems) and collected on BOND Plus Slides (Leica Microsystems).

The results show that hNCAM+/TH+ neurons were present in all 3 groups 1-month post-transplantation (FIG. 7a-c), suggesting that the NPCs are capable of differentiating into mature neurons and surviving in the host environment. However, no signs of engraftment were evident in the asF-iPS (FIG. 7h) or MC23-iPS (data not shown) groups. hNCAM/TH+ fibers can be seen extending from neurons in the graft core of the EC23-CLiPS group along axonal tracts of the corpus callosum (FIG. 7d and FIG. 7e). The immunostaining for the microglia/macrophages-specific marker Iba1 revealed an abundance of microglia/macrophage in the injected hemisphere compared to the non-injected hemisphere (FIG. 7i and FIG. 7j). Microglia/macrophages that infiltrated into the core of the graft displayed an amoeboid morphology characteristic of activated microglia compared to those at the periphery of the grafts which showed a ramified morphology typical of quiescent cells. In addition, infiltrated microglia stained positively for CD68, a marker for activated microglia. At 1-month post-transplantation, no accumulation of microglia was observed at injected sites of asF5-iPS- and MC23-CLiPS NPC transplanted brains, presumably because they have dispersed and returned to a quiescent state following clearance of the xenografts. Human TH+ neurons survived up to 9 months in some animals transplanted with EC23-CLiPS NPCs as confirmed by human nuclear antigen (HuNu) and human NCAM staining (FIG. 8a-f). The rotation asymmetry test revealed that lesioned animals will exhibit contraversive rotations due to hypersensitivity of post-synaptic D2 dopamine receptors on the lesioned striatum as a result of dopamine depletion, when challenged with the dopamine agonist apomorphine. Efficacy of administered interventions will be manifested as amelioration of this rotation asymmetry. Only animals transplanted with EC23-CLiPS NPCs showed improvement in rotational behaviour for both species in contrast to asF-iPS NPC or sham transplanted animals (FIG. 8h). In these mice, the reduction in rotations reached significance (p<0.05) beginning at Week 20 post-transplantation, decreasing to 18.2±24.7% and 11.1±20.8% at Week 20 and Week 22, respectively. The models showed latency in recovery, with initial observed worsening following transplantation. This is likely due the inflammatory reaction resulting from stereotaxic injection and time required for NPCs to mature, integrate with and innervate host tissues. Functional improvement of parkinsonian motor symptoms in EC23-CLiPS NPC transplanted animals suggests functional recovery of dopaminergic functions in the grafted striatum. To further investigate this, we performed PET imaging with the dopamine transporter (DAT) ligand [18F]FE-PE2I (Bang, J. I., et al., Nucl Med Biol, 2016. 43(2): p. 158-64; Sasaki, T., et al., J Nucl Med, 2012. 53(7): p. 1065-73) in transplanted mice. DAT is a presynaptic transmembrane protein primarily responsible for the reuptake of dopamine released at synapses and molecular imaging of DAT is an established tool for studying dopaminergic functions. PET imaging at 6-months post-transplantation showed recovery of DAT activity in grafted lesioned hemispheres to about 71.4±10.3% (n=3) the activity of non-lesioned hemispheres in EC23-iPS NPC transplanted mice (FIG. 8i). In contrast, the recovery was only 16.4±4.0% in asF-iPS-NPC transplanted mice. These results clearly indicate a significant restoration of dopamine reuptake function in EC23-iPS NPC grafted mice.

### Example 13: Transplantation of CLiPS-Derived Dopaminergic Neurons in Fully Immunocompetent Rodent Rat Models of Parkinson's Disease

Our transplantation results indicate that EC23-CLiPS-derived NPCs are tolerated when transplanted into the striatum of C56BL/6 mice. To rule out possible species-specific bias of this phenomenon, the transplantation study was replicated in a different species, the Wistar rat. Parkinsonism was induced in these rats by the injection of 6-OHDA into the MFB to lesion the nigrostriatal pathway. In this context, it is noted that all animal experiments were conducted according to protocols approved by the Institutional Animal Care and Use Committee (IACUC) of the National Neuroscience Institute (NNI), Singapore. Additional approval for rat experiments was provided by the IACUC of the National Technological University (NTU), Singapore. MFB lesions are known to cause a more complete depletion of the dopamine system compared to striatal lesions and are therefore presumed to be less likely to lead to spontaneous recovery (Tones, E. M. and S. B. Dunnett, Animal Models of Movement Disorders: Volume I, E. L. Lane and S. B. Dunnett, Editors. 2012, Humana Press: Totowa, N.J. p. 267-279). The rats were fully immunocompetent and no immunosuppression was administered prior to or following transplantation. For the analysis, female Wistar rats of ˜8 weeks old were purchased from InVivos Pte Ltd. Unilateral lesion was induced by stereotaxic injection of 20 μg of 6-OHDA in 4 μl into the left medial forebrain bundle (MFB) at the following coordinates: AP −4.4 mm; ML −1.2 mm; and DV −8.6 mm from dura. To determine suitable models for transplantation, apomorphine-induced rotations were scored as described in Example 12. Rats displaying >6 rotations/min were transplanted with 3 μl of about 1.25×10⁵ cells/μ1 Day 25 dopaminergic progenitors into the left striatum at the following coordinates with reference to Bregma: AP +0.8 mm; ML −2.5 mm; and DV −5 mm from dura. To assess whether transplanted NPCs can integrate and mediate functional benefits in lesioned animals, rotation asymmetry tests were performed at 1-monthly intervals as described in Example 12. Rats were sacrificed at 6 months by terminal anesthesia and brains were harvested for immunohistological analyses after transcardial perfusion with 4% PFA as described in Example 12. A few animals that failed the lesioning criteria were also similarly transplanted and sacrificed at 1 and 3 months post-transplantation to assess cell survival and engraftment.

The results show unilateral depletion of dopaminergic neurons in the substantia nigra as a result of retrograde transport of 6-OHDA via the MFB was confirmed in the model by DAB staining for TH in midbrain sections (FIG. 11d). Animals displaying at least 5 turns/minute under apomorphine challenge were transplanted with asF-iPS-, EC23-CLiPS- and MC23-CLiPS-derived NPCs. Histological analyses 3 months post-transplantation showed the presence of hCyto+/HuNu+ and hNCAM+/TH+ cells only in the EC23-CLiPS group. In addition, TH+ neurons in the graft showed expression of Synapsin 1, suggesting integration with host neurons (FIG. 11b). Further, only animals transplanted with EC23-CLiPS NPCs showed improvement in rotational behaviour for both species in contrast to asF-iPS NPC or sham transplanted animals (FIG. 11e). Also the rat models showed latency in recovery, with initial observed worsening following transplantation. This is likely due the inflammatory reaction resulting from stereotaxic injection and time required for NPCs to mature, integrate with and innervate host tissues. The results also indicate a significant restoration of dopamine reuptake function in CLiPS-derived NPC grafted rats.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The inventions illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention. The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group. Further embodiments of the invention will become apparent from the following claims.

What is claimed is:

1. A method of generating an induced pluripotent stem cell, wherein the method comprises expressing exogenous nucleic acids encoding the proteins OCT3/4, SOX2, KLF4, LIN28 and L-MYC and the p53-shRNA in a stem cell of the amniotic membrane of the umbilical cord under conditions suitable to reprogram the stem cell, thereby generating the induced pluripotent stem cell.

2. The method of claim 1, wherein the stem cell of the amniotic membrane of the umbilical cord is a mesenchymal stem cell of the amniotic membrane of the umbilical cord or an epithelial stem cell of the amniotic membrane of the umbilical cord.

3. The method of claim 1 or 2, wherein the mesenchymal stem of the amniotic membrane of the umbilical cord is a mesenchymal stem cell population, wherein at least about 90% or more cells of the stem cell population express each of the following markers: CD73, CD90 and CD105.

4. The method of claim 3, wherein at least about 90% or more cells of the mesenchymal stem cell population lack expression of the following markers: CD34, CD45 and HLA-DR.

5. The method of any one of claim 3 or 4, wherein at least about 91% or more, about 92% or more, about 93% or more, about 94% or more, about 95% or more, about 96% or more, about 97% or more, about 98% or more about 99% or more cells of the mesenchymal stem cell population express each of CD73, CD90 and CD105 and lack expression of each of CD34, CD45 and HLA-DR.

6. The method of claim 1, wherein the exogenous nucleic acids encoding the proteins OCT3/4, SOX2, KLF4, LIN28 and L-MYC and the p53-shRNA are provided by one, two or three vectors, wherein preferably a first vector encodes the protein OCT3/4 and the 53-shRNA, a second vector encodes the proteins SOX2 and KLF4 and a third vector encodes the proteins L-MYC and LIN28.

7. The method of any one of claims 1 to 6, wherein the stem cell of the amniotic membrane of the umbilical cord is subjected to transfection to transfer the exogenous nucleic acids into the stem cell.

8. The method of claim 7, wherein the stem cell of the amniotic membrane of the umbilical cord is subjected to electroporation to transfer the exogenous nucleic acids into the stem cell.

9. The method of claim 8, wherein the mesenchymal stem cell of the amniotic membrane of the umbilical cord is subjected to electroporation with 1 pulse having a duration time of about 15-25 ms and a voltage of about 1550-1650V, preferably to electroporation with 1 pulse having a duration time of about 20 ms and a voltage of about 1600V.

10. The method of claim 9, wherein the ratio of the amount of vector (plasmid) DNA for each vector to the number of mesenchymal stem cells of the amniotic membrane of the umbilical cord subjected to electroporation is in the range of about 1.5 μg plasmid DNA to about 1×10⁶ CLMC to of about 2.5 μg DNA to about 1×10⁶ CLMC, wherein the ratio is, for example, about 2.5 μg plasmid DNA:1×10⁶ cells, about 2.25 μg plasmid DNA:1×10⁶ cells, about 1.8 μg plasmid DNA:1×10⁶ cells, about 1.7 μg plasmid DNA:1×10⁶ cells, about 1.6 μg plasmid DNA:1×10⁶ cells, about 1.5 μg plasmid DNA:1×10⁶ cells, or preferably about 1.67:1×10⁶ cells.

11. The method of claim 8, wherein the epithelial stem cell of the amniotic membrane of the umbilical cord is subjected to electroporation with 2 pulses having a duration time of about 25-35 ms and a voltage of about 1300-1400V, preferably to electroporation with 2 pulses having a duration time of about 30 ms and a voltage of about 1350V.

12. The method of claim 11, wherein the ratio of the amount of vector (plasmid) DNA for each vector to the number of epithelial stem cells of the amniotic membrane of the umbilical cells subjected to electroporation is in the range of about 1.5 μg DNA to about 1×10⁶ cells to about 2.5 μg DNA to about 1×10⁶ cells, wherein the ratio is, for example, about 1.5 μg plasmid DNA:1×10⁶ cells, about 1.6 μg plasmid DNA:1×10⁶ cells, about 1.7 μg plasmid DNA:1×10⁶ cells, about 1.8 μg plasmid DNA:1×10⁶ cells, about 1.9 μg plasmid DNA:1×10⁶ cells, about 2.0 μg plasmid DNA:1×10⁶ cells, about 2.5 μg plasmid DNA:1×10⁶ cells, preferably about 1.67 μg plasmid DNA:1×10⁶ cells.

13. The method of any one of claims 7 to 12, wherein the transfected stem cell is cultivated in a medium suitable for cell recovery.

14. The method of claim 13, wherein the medium suitable for cell recovery is a serum-free medium.

15. The method of claim 13, wherein the medium suitable for the recovery of a transfected mesenchymal stem cell of the amniotic membrane of the umbilical cord consists of about 85 to 95% (v/v) defined medium and 5 to 15% (v/v) fetal bovine serum.

16. The medium of claim 15, wherein the medium suitable for the recovery of a transfected mesenchymal stem cell of the amniotic membrane of the umbilical cord consists of about 90% (v/v) chemically defined medium and about 10% (v/v) fetal bovine serum.

17. The medium of any of claim 14 or 15, wherein the medium contains about 85 to 95% (v/v) CMRL 1066 and about 5 to 15% (v/v) FBS.

18. The method of claim 13 or 14, wherein the medium suitable for the recovery of a transfected epithelial stem cell of the amniotic membrane of the umbilical cord comprises Mammary Epithelial Basal Medium MCDB 170, EpiLife medium, DMEM (Dulbecco's modified eagle medium), F12 (Ham's F12 Medium) and FBS (Fetal Bovine Serum).

19. The method of claim 18, wherein the medium suitable for the recovery of a transfected epithelial stem cell of the amniotic membrane of the umbilical cord comprises Mammary Epithelial Basal Medium MCDB 170 in a final concentration of about 10 to about 30% (v/v), EpiLife medium in a final concentration of about 20 to about 40% (v/v), F12 in a final concentration of about 5 to about 15% (v/v), DMEM in a final concentration of about 30 to about 45% (v/v) and FBS in a final concentration of about 0.1 to 2% (v/v).

20. The method of claim 19, wherein the medium suitable for the recovery of a transfected epithelial stem cell of the amniotic membrane of the umbilical cord comprises Mammary Epithelial Basal Medium MCDB 170 in a final concentration of about 15 to about 25% (v/v), EpiLife medium in a final concentration of about 25 to about 35% (v/v), F12 in a final concentration of about 7.5 to about 13% (v/v), DMEM in a final concentration of about 35 to about 40% (v/v) and FBS in a final concentration of about 0.5 to 1.5% (v/v).

21. The method of claim 20, wherein the medium suitable for the recovery of a transfected epithelial stem cell of the amniotic membrane of the umbilical cord comprises Mammary Epithelial Basal Medium MCDB 170 in a final concentration of about 20% (v/v), EpiLife medium in a final concentration of about 30% (v/v), F12 in a final concentration of about 12.5 (v/v), DMEM in a final concentration of about 37.5% (v/v) and FBS in a final concentration of about 1.0% (v/v).

22. The method of any of claims 18 to 21, wherein the medium suitable for the recovery of a transfected epithelial stem cell of the amniotic membrane of the umbilical cord is obtained by mixing to obtain a final volume of 1000 ml culture medium:
200 ml Mammary Epithelial Basal Medium MCDB 170
300 ml EpiLife medium
250 ml DMEM
250 ml DMEM/F12
1% Fetal Bovine Serum.

23. The method of any of claims 18 to 22, wherein the medium suitable for the recovery of a transfected epithelial stem cell of the amniotic membrane of the umbilical cord comprises insulin in a final concentration of about 1 to about 7.5 μg/ml.

24. The method of any of claims 18 to 24, wherein the medium suitable for the recovery of a transfected epithelial stem cell of the amniotic membrane of the umbilical cord comprises human epidermal growth factor in a final concentration of about 1 to about 15 ng/ml.

25. The method of any of claims 18 to 25, wherein the medium suitable for the recovery of a transfected epithelial stem cell of the amniotic membrane of the umbilical cord further comprises at least one of the following supplements: adenine, hydrocortisone, and 3,3′,5-Triiodo-L-thyronine sodium salt (T3).

26. The method of claim 25, wherein the medium suitable for the recovery of a transfected epithelial stem cell of the amniotic membrane of the umbilical cord comprises all three of adenine, hydrocortisone, and 3,3′,5-Triiodo-L-thyronine sodium salt (T3).

27. The method of any of claims 18 to 26, wherein the medium suitable for the recovery of a transfected epithelial stem cell of the amniotic membrane of the umbilical cord further comprises one of more Transforming Growth Factors (TGF).

28. The method of claim 27, wherein the medium comprises Transforming Growth Factor beta (TGF-beta) and/or transforming growth factor alpha.

29. The method of any of claims 18 to 28, wherein the medium suitable for the recovery of a transfected epithelial stem cell of the amniotic membrane of the umbilical cord further comprises Cholera Toxin from Vibrio cholerae.

30. The method of any one of claims 14 to 29, wherein the medium suitable for cell recovery contains a compound suppressing inflammatory response and enhancing cell survival.

31. The method of claim 30, wherein the compound is a glucocorticoid.

32. The method of claim 31, wherein the glucocorticoid is selected from the group consisting of prednisolone, methylprednisolone, dexamethasone, betamethasone, corticosterone and hydrocortisone.

33. The method of claim 31 or 32, wherein the hydrocortisone concentration is about 0.5 μM to about 2 μM.

34. The method of any one of claims 13 to 33, wherein the cultivation is carried out in a coated cell culture vessel, wherein the cell culture vessel is preferably coated with a serum-derived substrate or a serum-free substrate.

35. The method of any one of claims 9 to 36, wherein the medium suitable for cell recovery is replaced with a mixture of two different cell culture media about 1, 2 or 3 days after transfection, preferably about 2 days after transfection, thereby yielding an induced pluripotent stem cell colony.

36. The method of claim 35, wherein the two different cell culture media are the medium suitable for cell recovery and a second cell culture medium.

37. The method of claim 35 or 36, wherein the two different cell culture media are mixed in a ratio of about 1:1 (v/v) prepared by contacting 1 volume medium suitable for cell recovery to 1 volume second cell culture medium.

38. The method of claim 36 or 37, wherein the second cell culture medium is a maintenance medium for cultivation of induced pluripotent stem cells, wherein the medium is preferably selected from the group consisting of mTeSR1, StemMACS™ iPS-Brew XF, TeSR™ E8, mTeSR™ Plus, TeSR™2, mTeSR™1, Corning® NutriStem® hPSC XF Medium, Essential 8 Medium, StemFlex, StemFit Basic02 and PluriSTEM.

39. The method of any one of claims 35 to 38, wherein the mixture of cell culture media is replaced with the same mixture of cell culture media within about 3, 4 or 5 days after transfection, preferably about 4 days after transfection.

40. The method of any one of claims 35 to 39, wherein the mixture of cell culture media is replaced with the second cell culture medium within about 5, 6 or 7 days after transfection, preferably about 6 days after transfection.

41. The method of claim 40, wherein the second cell culture medium is changed daily or every second day, third day, preferably every second day.

42. The method of claim 40 or 41, wherein an induced pluripotent stem cell colony is selected when reaching a size of about 0.5 mm to about 1.5 mm in diameter, and the selected induced pluripotent stem colony is transferred to a coated cell culture vessel for cultivation and proliferation.

43. The method of claim 42, wherein the induced pluripotent stem cell colony is selected under bright field microscopy.

44. The method of claim 42 or 43, wherein the cell culture medium is changed daily or every second day, preferably every day.

45. The method of any one of claims 43 to 44, wherein the induced pluripotent stem cell colony is detached from the coated cell culture device when reaching a confluence of about 50%.

46. The method of claim 45, wherein the induced pluripotent stem cell colony is detached with a reagent selected from the group consisting of dissociation reagent, a dispase or an EDTA solution.

47. The method of claim 45 or 46, wherein a cell population formed from the induced pluripotent stem cell colony is passaged when reaching about 60-90% confluence, preferably when reaching 70-80% confluence.

48. The method of claim 47, wherein the cell population formed from the induced pluripotent stem cell colony is passaged in a ratio of about 1:3 (v/v), wherein the passaging in a ratio of about 1:3 (v/v) is performed by dividing about 1 volume dissociated induced pluripotent stem cells into about 2 volumes of dissociated induced pluripotent stem cells.

49. The method of claim 47 or 48, wherein the cell population formed from the induced pluripotent stem cell colony is dissociated with about 0.5 mM EDTA for passaging.

50. The method of claim 48 or 49, wherein the passaged cell population formed from the induced pluripotent stem cell colony is cultivated in a medium containing a substance enhancing the survival of the induced pluripotent stem cell.

51. The method of claim 50, wherein the substance enhancing the survival of the induced pluripotent stem cell colony is a ROCK inhibitor.

52. An induced pluripotent stem cell population obtainable by the method as defined in any of claims 1 to 51.

53. An induced pluripotent stem cell population obtained by the method as defined in any of claims 1 to 51.

54. A pharmaceutical composition comprising an induced pluripotent stem cell as defined in claim 52 or 53.

55. A method of differentiating an induced pluripotent stem cell as defined in claim 52 or 53 into a target cell, wherein the induced pluripotent stem cell is differentiated into the target cell under conditions suitable for differentiation.

56. The method of claim 55, wherein the target cell is selected from the group consisting of a dopaminergic neuronal cell, an oligodentrocyte, a hepatocyte, a cardiomyocyte, a hematopoietic progenitor cell, a blood cell, a neuronal cell, a motor neuron, a cartilage cell, a muscle cell, a bone cell, a dental cell, a hair follicle cell, an inner ear hair cell, a skin cell, a melanocyte, an immune cell, an astrocyte, a reproductive cell, a corneal cell, an intestinal cell, a lung cell, a kidney cell, a stomach cell, a mesenteric cell, and a fat cell.

57. The method of claim 56, wherein the immune cell is selected from the group consisting of a T-lympocyte, a B-lymphocyte, a microglia, and a natural killer cell.

58. The method of claim 56, wherein the induced pluripotent stem cell is cultivated in a medium adapted for proliferation and differentiation of the induced pluripotent stem cell into a dopaminergic neuronal cell.

59. The method of claim 56, wherein the induced pluripotent stem cell is cultivated in a medium adapted for proliferation and differentiation of the induced pluripotent stem cell into a hepatocyte.

60. The method of claim 56, wherein the induced pluripotent stem cell is cultivated in a medium adapted for proliferation and differentiation of the induced pluripotent stem cell into a cardiomyocyte.

61. The method of claim 60, wherein the induced pluripotent stem cell is cultivated in a medium adapted for proliferation and differentiation of the induced pluripotent stem cell into an oligodentrocyte.

62. A pharmaceutical composition comprising a differentiated induced pluripotent stem cell obtained by the method as defined in claims 56 to 61.

63. The pharmaceutical composition of claim 62, wherein the pharmaceutical composition is adapted for parenteral application.

64. A method of treating a congenital or acquired degenerative disorder in a subject, comprising administering to a subject a target cell differentiated from pluripotent stem cell by the method as defined in claims 56 to 61.

65. The method of claim 64, wherein the disorder is a neural disorder.

66. The method of claim 65, wherein the disease is neural disorder is selected from the group consisting of Parkinson's disease, Alzheimer's disease, Huntington's disease, Amyotrophic lateral sclerosis, multiple sclerosis and batten disease.

67. The method of claim 64, wherein the disorder is a hepatic disorder.

68. An extracellular membranous vesicle produced by an induced pluripotent stem cell population as defined in claim 52 or 53 or produced by a cell obtained by differentiation of an induced pluripotent stem cell as defined in claim 52 or 53.

69. The extracellular membranous vesicle of claim 68, wherein the vesicle is an exosome.

70. The use of an extracellular membranous vesicle as defined in claim 68 or 69 as delivery carrier of a therapeutic agent.

71. A cell culture medium comprising Mammary Epithelial Basal Medium MCDB 170, EpiLife medium, DMEM (Dulbecco's modified eagle medium), F12 (Ham's F12 Medium) and FBS (Fetal Bovine Serum).

72. The cell culture medium of claim 71, wherein the medium comprises Mammary Epithelial Basal Medium MCDB 170 in a final concentration of about 10 to about 30% (v/v), EpiLife medium in a final concentration of about 20 to about 40% (v/v), F12 in a final concentration of about 5 to about 15% (v/v), DMEM in a final concentration of about 30 to about 45% (v/v) and FBS in a final concentration of about 0.1 to 2% (v/v).

73. The cell culture medium of claim 72, wherein the medium comprises Mammary Epithelial Basal Medium MCDB 170 in a final concentration of about 15 to about 25% (v/v), EpiLife medium in a final concentration of about 25 to about 35% (v/v), F12 in a final concentration of about 7.5 to about 13% (v/v), DMEM in a final concentration of about 35 to about 40% (v/v) and FBS in a final concentration of about 0.5 to 1.5% (v/v).

74. The cell culture medium of claim 73, wherein the medium comprises Mammary Epithelial Basal Medium MCDB 170 in a final concentration of about 20% (v/v), EpiLife medium in a final concentration of about 30% (v/v), F12 in a final concentration of about 12.5 (v/v), DMEM in a final concentration of about 37.5% (v/v) and FBS in a final concentration of about 1.0% (v/v).

75. The cell culture medium of any of claims 71 to 74, wherein the medium is obtained by mixing to obtain a final volume of 1000 ml culture medium:
200 ml Mammary Epithelial Basal Medium MCDB 170,
300 ml EpiLife medium,
250 ml DMEM,
250 ml DMEM/F12, and
1% Fetal Bovine Serum.

76. The cell culture medium of any of claims 71 to 75, wherein the medium comprises insulin in a final concentration of about 1 to about 7.5 μg/ml.

77. The cell culture medium of any of claims 71 to 76, wherein the medium comprises human epidermal growth factor (EGF) in a final concentration of about 1 to about 15 ng/ml.

78. The cell culture medium of any of claims 71 to 77, wherein the medium suitable for the recovery of a transfected epithelial stem cell of the amniotic membrane of the umbilical cord comprises at least one of the following supplements: adenine, hydrocortisone, and 3,3′,5-Triiodo-L-thyronine sodium salt (T3).

79. The cell culture medium of claim 78, wherein the medium comprises all three of adenine, hydrocortisone, and 3,3′,5-Triiodo-L-thyronine sodium salt (T3).

80. The cell culture medium of claim 79, wherein the culture medium comprises adenine in a final concentration of about 0.05 to about 0.1 mM adenine, hydrocortisone in a final concentration of about 0.1 to 0.5 μM hydrocortisone and/or 3,3′,5-Triiodo-L-thyronine sodium salt (T3) in a final concentration of about 0.1 to about 5 ng/ml.

81. The cell culture medium of any of claims 71 to 80, wherein the medium comprises one of more Transforming Growth Factors (TGF).

82. The cell culture medium of claim 81, wherein the medium comprises Transforming Growth Factor beta 1 (TGF-beta 1) in a final concentration of about 0.1 to about 5 ng/ml and/or transforming growth factor alpha (TGF-alpha) in a final concentration of about 1.0 to about 10 ng/ml.

83. The medium of any of claims 71 to 82, wherein the medium comprises Cholera Toxin from Vibrio cholerae in a final concentration of about 1×10⁻¹¹ M to about 1×10⁻¹⁰ M.


SEQUENCE LISTING (50 sequences)

SEQ ID NO: 1 - DNA, 741 bp, Homo sapiens
ttctttttcctacagctcctgggcaacgtgctggttattgtgctgtctcatcattttggcaaagaattcgcccttcaccatggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatg

SEQ ID NO: 2 - protein, 352 aa, Homo sapiens
MAGHLASDFAFSPPPGGGDGSAGLEPGWVDPRTWLSFQGPPGGPGIGPGSEVLGISPCPPAYEFCGGMAYCGPQVGLGLVPQVGVETLQPEGQAGARVESNSEGTSSEPCADRPNAVKLEKVEPTPEESQDMKALQKELEQFAKLLKQKRITLGYTQADVGLTLGVLFGKVFSQTTICRFEALQLSLKNMCKLRPLLEKWVEEADNNENLQEICKSETLVQARKRKRTSIENRVRWSLETMFLKCPKPSLQQITHIANQLGLEKDVVRVWFCNRRQKGKRSSIEYSQREEYEATGTPFPGGAVSFPLPPGPHFGTPGYGSPHFTTLYSVPFPEGEAFPSVPVTALGSPMHSN

SEQ ID NO: 3 - DNA, 949 bp, Homo sapiens
ccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgcccttcaccatgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggc

SEQ ID NO: 4 - protein, 317 aa, Homo sapiens
MYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 5 - DNA, 1007 bp, Homo sapiens
ccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgggggatctcaaaattgtcgctcctgtcaaacaaactcttaactttgatttactcaaactggctggggatgtagaaagcaatccaggtccactcatgaacaattcgcccttcaccatggctgtcagtgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacga

SEQ ID NO: 6 - protein, 513 aa, Homo sapiens
MRQPPGESDMAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQGQSRGFVARAGEPCVCWPHFGTHGMMLTPPSSPLELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 7 - DNA, 679 bp, Homo sapiens
cttcaccatggactacgactcgtaccagcactatttctacgactatgactgcggggaggatttctaccgctccacggcgcccagcgaggacatctggaagaaattcgagctggtgccatcgccccccacgtcgccgccctggggcttgggtcccggcgcaggggacccggcccccgggattggtcccccggagccgtggcccggagggtgcaccggagacgaagcggaatcccggggccactcgaaaggctggggcaggaactacgcctccatcatacgccgtgactgcatgtggagcggcttctcggcccgggaacggctggagagagctgtgagcgaccggctcgctcctggcgcgccccgggggaacccgcccaaggcgtccgccgccccggactgcactcccagcctcgaagccggcaacccggcgcccgccgccccctgtccgctgggcgaacccaagacccaggcctgctccgggtccgagagcccaagcgactcggagaatgaagaaattgatgttgtgacagtagagaagaggcagtctctgggtattcggaagccggtcaccatcacggtgcgagcagaccccctggatccctgcatgaagcatttccacatctccatccatcagcaacagcacaactatgctgcccgttttcctccagaaagctgctcc

SEQ ID NO: 8 - protein, 364 aa, Homo sapiens
MDYDSYQHYFYDYDCGEDFYRSTAPSEDIWKKFELVPSPPTSPPWGLGPGAGDPAPGIGPPEPWPGGCTGDEAESRGHSKGWGRNYASIIRRDCMWSGFSARERLERAVSDRLAPGAPRGNPPKASAAPDCTPSLEAGNPAPAAPCPLGEPKTQACSGSESPSDSENEEIDVVTVEKRQSLGIRKPVTITVRADPLDPCMKHFHISIHQQQHNYAARFPPESCSQEEASERGPQEEVLERDAAGEKEDEEDEEIVSPPPVESEAAQSCHPKPVSSDTEDVTKRKNHNFLERKRRNDLRSRFLALRDQVPTLASCSKAPKVVILSKALEYLQALVGAEKRMATEKRQLRCRQQQLQKRIAYLTGY

SEQ ID NO: 9 - DNA, 901 bp, Homo sapiens
ccccctggatccctgcatgaagcatttccacatctccatccatcagcaacagcacaactatgctgcccgttttcctccagaaagctgctcccaagaagaggcttcagagaggggtccccaagaagaggttctggagagagatgctgcaggggaaaaggaagatgaggaggatgaagagattgtgagtcccccacctgtagaaagtgaggctgcccagtcctgccaccccaaacctgtcagttctgatactgaggatgtgaccaagaggaagaatcacaacttcctggagcgcaagaggcggaatgacctgcgttcgcgattcttggcgctgagggaccaggtgcccaccctggccagctgctccaaggcccccaaagtagtgatcctaagcaaggccttggaatacttgcaagccctggtgggggctgagaagaggatggctacagagaaaagacagctccgatgccggcagcagcagttgcagaaaagaattgcatacctcactggctacggagatctcaaaattgtcgctcctgtcaaacaaactcttaactttgatttactcaaactggctggggatgtagaaagcaatccaggtccactcatgaacaattcgcccttcaccatgggctccgtgtccaaccagcagtttgcaggtggctgcgccaaggcggcagaagaggcgcccgaggaggcgccggaggacgcggcccgggcggcggacgagcctcagctgctgcacggtgcgggcatctgtaagtggttcaacgtgcgcatggggttcggcttcctgtccatgaccgcccgcgccggggtcgcgctcgaccccccagtggatgtctttgtgcaccagagtaagctgcacatggaagggttccggagcttgaaggagggtgaggca

SEQ ID NO: 10 - protein, 209 aa, Homo sapiens
MGSVSNQQFAGGCAKAAEEAPEEAPEDAARAADEPQLLHGAGICKWFNVRMGFGFLSMTARAGVALDPPVDVFVHQSKLHMEGFRSLKEGEAVEFTFKKSAKGLESIRVTGPGGVFCIGSERRPKGKSMQKRRSKGDRCYNCGGLDHHAKECKLPPQPKKCHFCQSISHMVASCPLKAQQGPSAQGKPTYFREEEEEIHSPTLLPEAQN

SEQ ID NO: 11 - skipped

SEQ ID NO: 12 - DNA, 11681 bp, Homo sapiens
gtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcgggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgcccttcaccatggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctctgtcaccactctgggctctcccatgcattcaaactgaggtaagggcgaattcaagcttcggggactagtcatatgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcggtaaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatctcaattccgatcatattcaataacccttaatataacttcgtataatgtatgctatacgaagttattaggtctgaagaggagtttacgtccagccaagcttaggatcaattctcatgtttgacagcttatcatcgataagctgatcctcacaggccgcacccagcttttcttccgttgccccagtagcatctctgtctggtgaccttgaagaggaagaggaggggtcccgagaatccccatccctaccgtccagcaaaaagggggacgaggaatttgaggcctggcttgaggctcaggacgcaaatcttgaggatgttcagcgggagttttccgggctgcgagtaattggtgatgaggacgaggatggttcggaggatggggaattttcagacctggatctgtctgacagcgaccatgaaggggatgagggtgggggggctgttggagggggcaggagtctgcactccctgtattcactgagcgtcgtctaataaagatgtctattgatctcttttagtgtgaatcatgtctgacgaggggccaggtacaggacctggaaatggcctaggagagaagggagacacatctggaccagaaggctccggcggcagtggacctcaaagaagagggggtgataaccatggacgaggacggggaagaggacgaggacgaggaggcggaagaccaggagccccgggcggctcaggatcagggccaagacatagagatggtgtccggagaccccaaaaacgtccaagttgcattggctgcaaagggacccacggtggaacaggagcaggagcaggagcgggaggggcaggagcaggaggggcaggagcaggaggaggggcaggagcaggaggaggggcaggaggggcaggaggggcaggaggggcaggagcaggaggaggggcaggagcaggaggaggggcaggaggggcaggaggggcaggagcaggaggaggggcaggagcaggaggaggggcaggaggggcaggagcaggaggaggggcaggaggggcaggaggggcaggagcaggaggaggggcaggagcaggaggaggggcaggaggggcaggagcaggaggaggggcaggaggggcaggaggggcaggagcaggaggaggggcaggagcaggaggggcaggaggggcaggaggggcaggagcaggaggggcaggagcaggaggaggggcaggaggggcaggaggggcaggagcaggaggggcaggagcaggaggggcaggagcaggaggggcaggagcaggaggggcaggaggggcaggagcaggaggggcaggaggggcaggagcaggaggggcaggaggggcaggagcaggaggaggggcaggaggggcaggagcaggaggaggggcaggaggggcaggagcaggaggggcaggaggggcaggagcaggaggggcaggaggggcaggagcaggaggggcaggaggggcaggagcaggaggaggggcaggagcaggaggggcaggagcaggaggtggaggccggggtcgaggaggcagtggaggccggggtcgaggaggtagtggaggccggggtcgaggaggtagtggaggccgccggggtagaggacgtgaaagagccagggggggaagtcgtgaaagagccagggggagaggtcgtggacgtggagaaaagaggcccaggagtcccagtagtcagtcatcatcatccgggtctccaccgcgcaggccccctccaggtagaaggccatttttccaccctgtaggggaagccgattattttgaataccaccaagaaggtggcccagatggtgagcctgacgtgcccccgggagcgatagagcagggccccgcagatgacccaggagaaggcccaagcactggaccccggggtcagggtgatggaggcaggcgcaaaaaaggagggtggtttggaaagcatcgtggtcaaggaggttccaacccgaaatttgagaacattgcagaaggtttaagagctctcctggctaggagtcacgtagaaaggactaccgacgaaggaacttgggtcgccggtgtgttcgtatatggaggtagtaagacctccctttacaacctaaggcgaggaactgcccttgctattccacaatgtcgtcttacaccattgagtcgtctcccctttggaatggcccctggacccggcccacaacctggcccgctaagggagtccattgtctgttatttcatggtctttttacaaactcatatatttgctgaggttttgaaggatgcgattaaggaccttgttatgacaaagcccgctcctacctgcaatatcagggtgactgtgtgcagctttgacgatggagtagatttgcctccctggtttccacctatggtggaaggggctgccgcggagggtgatgacggagatgacggagatgaaggaggtgatggagatgagggtgaggaagggcaggagtgatgtaacttgttaggagacgccctcaatcgtattaaaagccgtgtattcccccgcactaaagaataaatccccagtagacatcatgcgtgctgttggtgtatttctggccatctgtcttgtcaccattttcgtcctcccaacatggggcaattgccggaacccttaatataacttcgtataatgtatgctatacgaagttattaggtccctcgaagaggttcactagcggatctcaattgggcatacccatgttgtcacgtcactcagctccgcgctcaacaccttctcgcgttggaaaacattagcgacatttacctggtgagcaatcagacatgcgacggctttagcctggcctccttaaattcacctaagaatgggagcaaccagcaggaaaaggacaagcagcgaaaattcacgcccccttgggaggtggcggcatatgcaaaggatagcactcccactctactactgggtatcatatgctgactgtatatgcatgaggatagcatatgctacccggatacagattaggatagcatatactacccagatatagattaggatagcatatgctacccagatatagattaggatagcctatgctacccagatataaattaggatagcatatactacccagatatagattaggatagcatatgctacccagatatagattaggatagcctatgctacccagatatagattaggatagcatatgctacccagatatagattaggatagcatatgctatccagatatttgggtagtatatgctacccagatataaattaggatagcatatactaccctaatctctattaggatagcatatgctacccggatacagattaggatagcatatactacccagatatagattaggatagcatatgctacccagatatagattaggatagcctatgctacccagatataaattaggatagcatatactacccagatatagattaggatagcatatgctacccagatatagattaggatagcctatgctacccagatatagattaggatagcatatgctatccagatatttgggtagtatatgctacccatggcaacattagcccaccgtgctctcagcgacctcgtgaatatgaggaccaacaaccctgtgcttggcgctcaggcgcaagtgtgtgtaatttgtcctccagatcgcagcaatcgcgcccctatcttggcccgcccacctacttatgcaggtattccccggggtgccattagtggttttgtgggcaagtggtttgaccgcagtggttagcggggttacaatcagccaagttattacacccttattttacagtccaaaaccgcagggcggcgtgtgggggctgacgcgtgcccccactccacaatttcaaaaaaaagagtggccacttgtctttgtttatgggccccattggcgtggagccccgtttaattttcgggggtgttagagacaaccagtggagtccgctgctgtcggcgtccactctctttccccttgttacaaatagagtgtaacaacatggttcacctgtcttggtccctgcctgggacacatcttaataaccccagtatcatattgcactaggattatgtgttgcccatagccataaattcgtgtgagatggacatccagtctttacggcttgtccccaccccatggatttctattgttaaagatattcagaatgtttcattcctacactagtatttattgcccaaggggtttgtgagggttatattggtgtcatagcacaatgccaccactgaaccccccgtccaaattttattctgggggcgtcacctgaaaccttgttttcgagcacctcacatacaccttactgttcacaactcagcagttattctattagctaaacgaaggagaatgaagaagcaggcgaagattcaggagagttcactgcccgctccttgatcttcagccactgcccttgtgactaaaatggttcactaccctcgtggaatcctgaccccatgtaaataaaaccgtgacagctcatggggtgggagatatcgctgttccttaggacccttttactaaccctaattcgatagcatatgcttcccgttgggtaacatatgctattgaattagggttagtctggatagtatatactactacccgggaagcatatgctacccgtttagggttaacaagggggccttataaacactattgctaatgccctcttgagggtccgcttatcggtagctacacaggcccctctgattgacgttggtgtagcctcccgtagtcttcctgggcccctgggaggtacatgtcccccagcattggtgtaagagcttcagccaagagttacacataaaggcaatgttgtgttgcagtccacagactgcaaagtctgctccaggatgaaagccactcagtgttggcaaatgtgcacatccatttataaggatgtcaactacagtcagagaacccctttgtgtttggtccccccccgtgtcacatgtggaacagggcccagttggcaagttgtaccaaccaactgaagggattacatgcactgccccgcgaagaaggggcagagatgtcgtagtcaggtttagttcgtccggggcggggcatcgatcctctagagtcgacgctagcggatccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgggccgactccagtggtaatctacttcaagagagtagattaccactggagtcttttttggaattcctgcagcccgggggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgg

SEQ ID NO: 13 - DNA, 12693 bp, Homo sapiens
gtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcgggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgcccttcaccatgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgggggatctcaaaattgtcgctcctgtcaaacaaactcttaactttgatttactcaaactggctggggatgtagaaagcaatccaggtccactcatgaacaattcgcccttcaccatggctgtcagtgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagagacatttttaaaagggcgaattgcctgcaggaattcaagcttcggggactagtcatatgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcggtaaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatctcaattccgatcatattcaataacccttaatataacttcgtataatgtatgctatacgaagttattaggtctgaagaggagtttacgtccagccaagcttaggatcaattctcatgtttgacagcttatcatcgataagctgatcctcacaggccgcacccagcttttcttccgttgccccagtagcatctctgtctggtgaccttgaagaggaagaggaggggtcccgagaatccccatccctaccgtccagcaaaaagggggacgaggaatttgaggcctggcttgaggctcaggacgcaaatcttgaggatgttcagcgggagttttccgggctgcgagtaattggtgatgaggacgaggatggttcggaggatggggaattttcagacctggatctgtctgacagcgaccatgaaggggatgagggtgggggggctgttggagggggcaggagtctgcactccctgtattcactgagcgtcgtctaataaagatgtctattgatctcttttagtgtgaatcatgtctgacgaggggccaggtacaggacctggaaatggcctaggagagaagggagacacatctggaccagaaggctccggcggcagtggacctcaaagaagagggggtgataaccatggacgaggacggggaagaggacgaggacgaggaggcggaagaccaggagccccgggcggctcaggatcagggccaagacatagagatggtgtccggagaccccaaaaacgtccaagttgcattggctgcaaagggacccacggtggaacaggagcaggagcaggagcgggaggggcaggagcaggaggggcaggagcaggaggaggggcaggagcaggaggaggggcaggaggggcaggaggggcaggaggggcaggagcaggaggaggggcaggagcaggaggaggggcaggaggggcaggaggggcaggagcaggaggaggggcaggagcaggaggaggggcaggaggggcaggagcaggaggaggggcaggaggggcaggaggggcaggagcaggaggaggggcaggagcaggaggaggggcaggaggggcaggagcaggaggaggggcaggaggggcaggaggggcaggagcaggaggaggggcaggagcaggaggggcaggaggggcaggaggggcaggagcaggaggggcaggagcaggaggaggggcaggaggggcaggaggggcaggagcaggaggggcaggagcaggaggggcaggagcaggaggggcaggagcaggaggggcaggaggggcaggagcaggaggggcaggaggggcaggagcaggaggggcaggaggggcaggagcaggaggaggggcaggaggggcaggagcaggaggaggggcaggaggggcaggagcaggaggggcaggaggggcaggagcaggaggggcaggaggggcaggagcaggaggggcaggaggggcaggagcaggaggaggggcaggagcaggaggggcaggagcaggaggtggaggccggggtcgaggaggcagtggaggccggggtcgaggaggtagtggaggccggggtcgaggaggtagtggaggccgccggggtagaggacgtgaaagagccagggggggaagtcgtgaaagagccagggggagaggtcgtggacgtggagaaaagaggcccaggagtcccagtagtcagtcatcatcatccgggtctccaccgcgcaggccccctccaggtagaaggccatttttccaccctgtaggggaagccgattattttgaataccaccaagaaggtggcccagatggtgagcctgacgtgcccccgggagcgatagagcagggccccgcagatgacccaggagaaggcccaagcactggaccccggggtcagggtgatggaggcaggcgcaaaaaaggagggtggtttggaaagcatcgtggtcaaggaggttccaacccgaaatttgagaacattgcagaaggtttaagagctctcctggctaggagtcacgtagaaaggactaccgacgaaggaacttgggtcgccggtgtgttcgtatatggaggtagtaagacctccctttacaacctaaggcgaggaactgcccttgctattccacaatgtcgtcttacaccattgagtcgtctcccctttggaatggcccctggacccggcccacaacctggcccgctaagggagtccattgtctgttatttcatggtctttttacaaactcatatatttgctgaggttttgaaggatgcgattaaggaccttgttatgacaaagcccgctcctacctgcaatatcagggtgactgtgtgcagctttgacgatggagtagatttgcctccctggtttccacctatggtggaaggggctgccgcggagggtgatgacggagatgacggagatgaaggaggtgatggagatgagggtgaggaagggcaggagtgatgtaacttgttaggagacgccctcaatcgtattaaaagccgtgtattcccccgcactaaagaataaatccccagtagacatcatgcgtgctgttggtgtatttctggccatctgtcttgtcaccattttcgtcctcccaacatggggcaattgccggaacccttaatataacttcgtataatgtatgctatacgaagttattaggtccctcgaagaggttcactagcggatctcaattgggcatacccatgttgtcacgtcactcagctccgcgctcaacaccttctcgcgttggaaaacattagcgacatttacctggtgagcaatcagacatgcgacggctttagcctggcctccttaaattcacctaagaatgggagcaaccagcaggaaaaggacaagcagcgaaaattcacgcccccttgggaggtggcggcatatgcaaaggatagcactcccactctactactgggtatcatatgctgactgtatatgcatgaggatagcatatgctacccggatacagattaggatagcatatactacccagatatagattaggatagcatatgctacccagatatagattaggatagcctatgctacccagatataaattaggatagcatatactacccagatatagattaggatagcatatgctacccagatatagattaggatagcctatgctacccagatatagattaggatagcatatgctacccagatatagattaggatagcatatgctatccagatatttgggtagtatatgctacccagatataaattaggatagcatatactaccctaatctctattaggatagcatatgctacccggatacagattaggatagcatatactacccagatatagattaggatagcatatgctacccagatatagattaggatagcctatgctacccagatataaattaggatagcatatactacccagatatagattaggatagcatatgctacccagatatagattaggatagcctatgctacccagatatagattaggatagcatatgctatccagatatttgggtagtatatgctacccatggcaacattagcccaccgtgctctcagcgacctcgtgaatatgaggaccaacaaccctgtgcttggcgctcaggcgcaagtgtgtgtaatttgtcctccagatcgcagcaatcgcgcccctatcttggcccgcccacctacttatgcaggtattccccggggtgccattagtggttttgtgggcaagtggtttgaccgcagtggttagcggggttacaatcagccaagttattacacccttattttacagtccaaaaccgcagggcggcgtgtgggggctgacgcgtgcccccactccacaatttcaaaaaaaagagtggccacttgtctttgtttatgggccccattggcgtggagccccgtttaattttcgggggtgttagagacaaccagtggagtccgctgctgtcggcgtccactctctttccccttgttacaaatagagtgtaacaacatggttcacctgtcttggtccctgcctgggacacatcttaataaccccagtatcatattgcactaggattatgtgttgcccatagccataaattcgtgtgagatggacatccagtctttacggcttgtccccaccccatggatttctattgttaaagatattcagaatgtttcattcctacactagtatttattgcccaaggggtttgtgagggttatattggtgtcatagcacaatgccaccactgaaccccccgtccaaattttattctgggggcgtcacctgaaaccttgttttcgagcacctcacatacaccttactgttcacaactcagcagttattctattagctaaacgaaggagaatgaagaagcaggcgaagattcaggagagttcactgcccgctccttgatcttcagccactgcccttgtgactaaaatggttcactaccctcgtggaatcctgaccccatgtaaataaaaccgtgacagctcatggggtgggagatatcgctgttccttaggacccttttactaaccctaattcgatagcatatgcttcccgttgggtaacatatgctattgaattagggttagtctggatagtatatactactacccgggaagcatatgctacccgtttagggttaacaagggggccttataaacactattgctaatgccctcttgagggtccgcttatcggtagctacacaggcccctctgattgacgttggtgtagcctcccgtagtcttcctgggcccctgggaggtacatgtcccccagcattggtgtaagagcttcagccaagagttacacataaaggcaatgttgtgttgcagtccacagactgcaaagtctgctccaggatgaaagccactcagtgttggcaaatgtgcacatccatttataaggatgtcaactacagtcagagaacccctttgtgtttggtccccccccgtgtcacatgtggaacagggcccagttggcaagttgtaccaaccaactgaagggattacatgcactgccccgcgaagaaggggcagagatgtcgtagtcaggtttagttcgtccggggcggggcatcgatcctctagagtcgacgctagcggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgg

SEQ ID NO: 14 - DNA, 12051 bp, Homo sapiens
gtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcgggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgcccttcaccatggactacgactcgtaccagcactatttctacgactatgactgcggggaggatttctaccgctccacggcgcccagcgaggacatctggaagaaattcgagctggtgccatcgccccccacgtcgccgccctggggcttgggtcccggcgcaggggacccggcccccgggattggtcccccggagccgtggcccggagggtgcaccggagacgaagcggaatcccggggccactcgaaaggctggggcaggaactacgcctccatcatacgccgtgactgcatgtggagcggcttctcggcccgggaacggctggagagagctgtgagcgaccggctcgctcctggcgcgccccgggggaacccgcccaaggcgtccgccgccccggactgcactcccagcctcgaagccggcaacccggcgcccgccgccccctgtccgctgggcgaacccaagacccaggcctgctccgggtccgagagcccaagcgactcggagaatgaagaaattgatgttgtgacagtagagaagaggcagtctctgggtattcggaagccggtcaccatcacggtgcgagcagaccccctggatccctgcatgaagcatttccacatctccatccatcagcaacagcacaactatgctgcccgttttcctccagaaagctgctcccaagaagaggcttcagagaggggtccccaagaagaggttctggagagagatgctgcaggggaaaaggaagatgaggaggatgaagagattgtgagtcccccacctgtagaaagtgaggctgcccagtcctgccaccccaaacctgtcagttctgatactgaggatgtgaccaagaggaagaatcacaacttcctggagcgcaagaggcggaatgacctgcgttcgcgattcttggcgctgagggaccaggtgcccaccctggccagctgctccaaggcccccaaagtagtgatcctaagcaaggccttggaatacttgcaagccctggtgggggctgagaagaggatggctacagagaaaagacagctccgatgccggcagcagcagttgcagaaaagaattgcatacctcactggctacggagatctcaaaattgtcgctcctgtcaaacaaactcttaactttgatttactcaaactggctggggatgtagaaagcaatccaggtccactcatgaacaattcgcccttcaccatgggctccgtgtccaaccagcagtttgcaggtggctgcgccaaggcggcagaagaggcgcccgaggaggcgccggaggacgcggcccgggcggcggacgagcctcagctgctgcacggtgcgggcatctgtaagtggttcaacgtgcgcatggggttcggcttcctgtccatgaccgcccgcgccggggtcgcgctcgaccccccagtggatgtctttgtgcaccagagtaagctgcacatggaagggttccggagcttgaaggagggtgaggcagtggagttcacctttaagaagtcagccaagggtctggaatccatccgtgtcaccggacctggtggagtattctgtattgggagtgagaggcggccaaaaggaaagagcatgcagaagcgcagatcaaaaggagacaggtgctacaactgtggaggtctagatcatcatgccaaggaatgcaagctgccaccccagcccaagaagtgccacttctgccagagcatcagccatatggtagcctcatgtccgctgaaggcccagcagggccctagtgcacagggaaagccaacctactttcgagaggaagaagaagaaatccacagccctaccctgctcccggaggcacagaattgaaagggcgaattgcctgcaggaattcaagcttcggggactagtcatatgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcggtaaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatctcaattccgatcatattcaataacccttaatataacttcgtataatgtatgctatacgaagttattaggtctgaagaggagtttacgtccagccaagcttaggatcaattctcatgtttgacagcttatcatcgataagctgatcctcacaggccgcacccagcttttcttccgttgccccagtagcatctctgtctggtgaccttgaagaggaagaggaggggtcccgagaatccccatccctaccgtccagcaaaaagggggacgaggaatttgaggcctggcttgaggctcaggacgcaaatcttgaggatgttcagcgggagttttccgggctgcgagtaattggtgatgaggacgaggatggttcggaggatggggaattttcagacctggatctgtctgacagcgaccatgaaggggatgagggtgggggggctgttggagggggcaggagtctgcactccctgtattcactgagcgtcgtctaataaagatgtctattgatctcttttagtgtgaatcatgtctgacgaggggccaggtacaggacctggaaatggcctaggagagaagggagacacatctggaccagaaggctccggcggcagtggacctcaaagaagagggggtgataaccatggacgaggacggggaagaggacgaggacgaggaggcggaagaccaggagccccgggcggctcaggatcagggccaagacatagagatggtgtccggagaccccaaaaacgtccaagttgcattggctgcaaagggacccacggtggaacaggagcaggagcaggagcgggaggggcaggagcaggaggggcaggagcaggaggaggggcaggagcaggaggaggggcaggaggggcaggaggggcaggaggggcaggagcaggaggaggggcaggagcaggaggaggggcaggaggggcaggaggggcaggagcaggaggaggggcaggagcaggaggaggggcaggaggggcaggagcaggaggaggggcaggaggggcaggaggggcaggagcaggaggaggggcaggagcaggaggaggggcaggaggggcaggagcaggaggaggggcaggaggggcaggaggggcaggagcaggaggaggggcaggagcaggaggggcaggaggggcaggaggggcaggagcaggaggggcaggagcaggaggaggggcaggaggggcaggaggggcaggagcaggaggggcaggagcaggaggggcaggagcaggaggggcaggagcaggaggggcaggaggggcaggagcaggaggggcaggaggggcaggagcaggaggggcaggaggggcaggagcaggaggaggggcaggaggggcaggagcaggaggaggggcaggaggggcaggagcaggaggggcaggaggggcaggagcaggaggggcaggaggggcaggagcaggaggggcaggaggggcaggagcaggaggaggggcaggagcaggaggggcaggagcaggaggtggaggccggggtcgaggaggcagtggaggccggggtcgaggaggtagtggaggccggggtcgaggaggtagtggaggccgccggggtagaggacgtgaaagagccagggggggaagtcgtgaaagagccagggggagaggtcgtggacgtggagaaaagaggcccaggagtcccagtagtcagtcatcatcatccgggtctccaccgcgcaggccccctccaggtagaaggccatttttccaccctgtaggggaagccgattattttgaataccaccaagaaggtggcccagatggtgagcctgacgtgcccccgggagcgatagagcagggccccgcagatgacccaggagaaggcccaagcactggaccccggggtcagggtgatggaggcaggcgcaaaaaaggagggtggtttggaaagcatcgtggtcaaggaggttccaacccgaaatttgagaacattgcagaaggtttaagagctctcctggctaggagtcacgtagaaaggactaccgacgaaggaacttgggtcgccggtgtgttcgtatatggaggtagtaagacctccctttacaacctaaggcgaggaactgcccttgctattccacaatgtcgtcttacaccattgagtcgtctcccctttggaatggcccctggacccggcccacaacctggcccgctaagggagtccattgtctgttatttcatggtctttttacaaactcatatatttgctgaggttttgaaggatgcgattaaggaccttgttatgacaaagcccgctcctacctgcaatatcagggtgactgtgtgcagctttgacgatggagtagatttgcctccctggtttccacctatggtggaaggggctgccgcggagggtgatgacggagatgacggagatgaaggaggtgatggagatgagggtgaggaagggcaggagtgatgtaacttgttaggagacgccctcaatcgtattaaaagccgtgtattcccccgcactaaagaataaatccccagtagacatcatgcgtgctgttggtgtatttctggccatctgtcttgtcaccattttcgtcctcccaacatggggcaattgccggaacccttaatataacttcgtataatgtatgctatacgaagttattaggtccctcgaagaggttcactagcggatctcaattgggcatacccatgttgtcacgtcactcagctccgcgctcaacaccttctcgcgttggaaaacattagcgacatttacctggtgagcaatcagacatgcgacggctttagcctggcctccttaaattcacctaagaatgggagcaaccagcaggaaaaggacaagcagcgaaaattcacgcccccttgggaggtggcggcatatgcaaaggatagcactcccactctactactgggtatcatatgctgactgtatatgcatgaggatagcatatgctacccggatacagattaggatagcatatactacccagatatagattaggatagcatatgctacccagatatagattaggatagcctatgctacccagatataaattaggatagcatatactacccagatatagattaggatagcatatgctacccagatatagattaggatagcctatgctacccagatatagattaggatagcatatgctacccagatatagattaggatagcatatgctatccagatatttgggtagtatatgctacccagatataaattaggatagcatatactaccctaatctctattaggatagcatatgctacccggatacagattaggatagcatatactacccagatatagattaggatagcatatgctacccagatatagattaggatagcctatgctacccagatataaattaggatagcatatactacccagatatagattaggatagcatatgctacccagatatagattaggatagcctatgctacccagatatagattaggatagcatatgctatccagatatttgggtagtatatgctacccatggcaacattagcccaccgtgctctcagcgacctcgtgaatatgaggaccaacaaccctgtgcttggcgctcaggcgcaagtgtgtgtaatttgtcctccagatcgcagcaatcgcgcccctatcttggcccgcccacctacttatgcaggtattccccggggtgccattagtggttttgtgggcaagtggtttgaccgcagtggttagcggggttacaatcagccaagttattacacccttattttacagtccaaaaccgcagggcggcgtgtgggggctgacgcgtgcccccactccacaatttcaaaaaaaagagtggccacttgtctttgtttatgggccccattggcgtggagccccgtttaattttcgggggtgttagagacaaccagtggagtccgctgctgtcggcgtccactctctttccccttgttacaaatagagtgtaacaacatggttcacctgtcttggtccctgcctgggacacatcttaataaccccagtatcatattgcactaggattatgtgttgcccatagccataaattcgtgtgagatggacatccagtctttacggcttgtccccaccccatggatttctattgttaaagatattcagaatgtttcattcctacactagtatttattgcccaaggggtttgtgagggttatattggtgtcatagcacaatgccaccactgaaccccccgtccaaattttattctgggggcgtcacctgaaaccttgttttcgagcacctcacatacaccttactgttcacaactcagcagttattctattagctaaacgaaggagaatgaagaagcaggcgaagattcaggagagttcactgcccgctccttgatcttcagccactgcccttgtgactaaaatggttcactaccctcgtggaatcctgaccccatgtaaataaaaccgtgacagctcatggggtgggagatatcgctgttccttaggacccttttactaaccctaattcgatagcatatgcttcccgttgggtaacatatgctattgaattagggttagtctggatagtatatactactacccgggaagcatatgctacccgtttagggttaacaagggggccttataaacactattgctaatgccctcttgagggtccgcttatcggtagctacacaggcccctctgattgacgttggtgtagcctcccgtagtcttcctgggcccctgggaggtacatgtcccccagcattggtgtaagagcttcagccaagagttacacataaaggcaatgttgtgttgcagtccacagactgcaaagtctgctccaggatgaaagccactcagtgttggcaaatgtgcacatccatttataaggatgtcaactacagtcagagaacccctttgtgtttggtccccccccgtgtcacatgtggaacagggcccagttggcaagttgtaccaaccaactgaagggattacatgcactgccccgcgaagaaggggcagagatgtcgtagtcaggtttagttcgtccggggcggggcatcgatcctctagagtcgacgctagcggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgg

SEQ ID NO: 15 - DNA, 21 bp, Homo sapiens
aaggtcccggtcaagaaacag

SEQ ID NO: 16 - DNA, 20 bp, Homo sapiens
cttctgcgtcacaccattgc

SEQ ID NO: 17 - DNA, 20 bp, Homo sapiens
cccacatgaagcgacttccc

SEQ ID NO: 18 - DNA, 21 bp, Homo sapiens
aggtccaggagatcgttgaac

SEQ ID NO: 19 - DNA, 20 bp, Homo sapiens
tggacagttacgcgcacatg

SEQ ID NO: 20 - DNA, 21 bp, Homo sapiens
gagtaggacatgctgtaggtg

SEQ ID NO: 21 - DNA, 20 bp, Homo sapiens
tgcggcccttgctgcagaag

SEQ ID NO: 22 - DNA, 20 bp, Homo sapiens
gctgctgggcgatgtggctg

SEQ ID NO: 23 - DNA, 22 bp, Homo sapiens
atgcattcaaactgaggtaagg

SEQ ID NO: 24 - DNA, 22 bp, Homo sapiens
tagcgtaaaaggagcaacatag

SEQ ID NO: 25 - DNA, 21 bp, Homo sapiens
ccatatggtagcctcatgtcc

SEQ ID NO: 26 - DNA, 21 bp, Homo sapiens
tcaattctgtgcctccgggag

SEQ ID NO: 27 - DNA, 23 bp, Homo sapiens
accacctcgccttacacatgaag

SEQ ID NO: 28 - DNA, 22 bp, Homo sapiens
tagcgtaaaaggagcaacatag

SEQ ID NO: 29 - DNA, 21 bp, Homo sapiens
ggctgagaagaggatggctac

SEQ ID NO: 30 - DNA, 21 bp, Homo sapiens
agtttgtttgacaggagcgac

SEQ ID NO: 31 - DNA, 20 bp, Homo sapiens
tcacatgtcccagcactacc

SEQ ID NO: 32 - DNA, 21 bp, Homo sapiens
agtttgtttgacaggagcgac

SEQ ID NO: 33 - DNA, 20 bp, Homo sapiens
aacccaagacccaggcctgc

SEQ ID NO: 34 - DNA, 20 bp, Homo sapiens
ggtctgctcgcaccgtgatg

SEQ ID NO: 35 - DNA, 20 bp, Homo sapiens
gaaatggcctaggagagaag

SEQ ID NO: 36 - DNA, 20 bp, Homo sapiens
cagccaatgcaacttggacg

SEQ ID NO: 37 - DNA, 24 bp, Homo sapiens
cttatgctacgtaaaggagctggg

SEQ ID NO: 38 - DNA, 24 bp, Homo sapiens
ttgtgccaacccaggtcccggaag

SEQ ID NO: 39 - DNA, 24 bp, Homo sapiens
tatcagatcctaaacagctcgcag

SEQ ID NO: 40 - DNA, 23 bp, Homo sapiens
cgtacgcaaattaaagtccagag

SEQ ID NO: 41 - DNA, 23 bp, Homo sapiens
actacaacgcctacgagtcctac

SEQ ID NO: 42 - DNA, 24 bp, Homo sapiens
gttgcaccagaaaagtcagagttg

SEQ ID NO: 43 - DNA, 24 bp, Homo sapiens
atatcccgccgtgggtgaaagttc

SEQ ID NO: 44 - DNA, 24 bp, Homo sapiens
actcagccatggactggagcatcc

SEQ ID NO: 45 - DNA, 26 bp, Homo sapiens
cctgctcaagctgactcgacaccgtg

SEQ ID NO: 46 - DNA, 25 bp, Homo sapiens
ggaaaagctggccctggggtggagc

SEQ ID NO: 47 - DNA, 22 bp, Homo sapiens
tgctgctcacagggcccgatac

SEQ ID NO: 48 - DNA, 23 bp, Homo sapiens
tcctttcgagctcagtgcaccac

SEQ ID NO: 49 - DNA, 21 bp, Homo sapiens
ctggcgctgagtacgtcgtgg

SEQ ID NO: 50 - DNA, 21 bp, Homo sapiens
gcagttggtggtgcaggaggc